SEQUENCE LISTING (38 sequences)

SEQ ID NO: 1 - protein, 105 aa, Artificial Sequence
  Synthetic
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 2 - protein, 5 aa, Artificial Sequence
  Synthetic
WCGPC

SEQ ID NO: 3 - protein, 4 aa, Artificial Sequence
  Synthetic
CGPC

SEQ ID NO: 4 - DNA, 318 bp, Artificial Sequence
  Synthetic
atggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaa

SEQ ID NO: 5 - protein, 24 aa, Artificial Sequence
  Synthetic
LVVVDFSATWCGPCKMIKPFFHSL

SEQ ID NO: 6 - protein, 23 aa, Artificial Sequence
  Synthetic
VVVDFSATWCGPCKMIKPFFHSL

SEQ ID NO: 7 - protein, 21 aa, Artificial Sequence
  Synthetic
VDFSATWCGPCKMIKPFFHSL

SEQ ID NO: 8 - protein, 20 aa, Artificial Sequence
  Synthetic
DFSATWCGPCKMIKPFFHSL

SEQ ID NO: 9 - protein, 23 aa, Artificial Sequence
  Synthetic
LVVVDFSATWCGPCKMIKPFFHS

SEQ ID NO: 10 - protein, 22 aa, Artificial Sequence
  Synthetic
VVVDFSATWCGPCKMIKPFFHS

SEQ ID NO: 11 - protein, 21 aa, Artificial Sequence
  Synthetic
VVDFSATWCGPCKMIKPFFHS

SEQ ID NO: 12 - protein, 20 aa, Artificial Sequence
  Synthetic
VDFSATWCGPCKMIKPFFHS

SEQ ID NO: 13 - protein, 19 aa, Artificial Sequence
  Synthetic
DFSATWCGPCKMIKPFFHS

SEQ ID NO: 14 - protein, 18 aa, Artificial Sequence
  Synthetic
FSATWCGPCKMIKPFFHS

SEQ ID NO: 15 - protein, 22 aa, Artificial Sequence
  Synthetic
LVVVDFSATWCGPCKMIKPFFH

SEQ ID NO: 16 - protein, 21 aa, Artificial Sequence
  Synthetic
VVVDFSATWCGPCKMIKPFFH

SEQ ID NO: 17 - protein, 20 aa, Artificial Sequence
  Synthetic
VVDFSATWCGPCKMIKPFFH

SEQ ID NO: 18 - protein, 19 aa, Artificial Sequence
  Synthetic
VDFSATWCGPCKMIKPFFH

SEQ ID NO: 19 - protein, 18 aa, Artificial Sequence
  Synthetic
DFSATWCGPCKMIKPFFH

SEQ ID NO: 20 - protein, 17 aa, Artificial Sequence
  Synthetic
FSATWCGPCKMIKPFFH

SEQ ID NO: 21 - protein, 21 aa, Artificial Sequence
  Synthetic
LVVVDFSATWCGPCKMIKPFF

SEQ ID NO: 22 - protein, 20 aa, Artificial Sequence
  Synthetic
VVVDFSATWCGPCKMIKPFF

SEQ ID NO: 23 - protein, 19 aa, Artificial Sequence
  Synthetic
VVDFSATWCGPCKMIKPFF

SEQ ID NO: 24 - protein, 18 aa, Artificial Sequence
  Synthetic
VDFSATWCGPCKMIKPFF

SEQ ID NO: 25 - protein, 17 aa, Artificial Sequence
  Synthetic
DFSATWCGPCKMIKPFF

SEQ ID NO: 26 - protein, 16 aa, Artificial Sequence
  Synthetic
FSATWCGPCKMIKPFF

SEQ ID NO: 27 - protein, 20 aa, Artificial Sequence
  Synthetic
LVVVDFSATWCGPCKMIKPF

SEQ ID NO: 28 - protein, 19 aa, Artificial Sequence
  Synthetic
VVVDFSATWCGPCKMIKPF

SEQ ID NO: 29 - protein, 18 aa, Artificial Sequence
  Synthetic
VVDFSATWCGPCKMIKPF

SEQ ID NO: 30 - protein, 17 aa, Artificial Sequence
  Synthetic
VDFSATWCGPCKMIKPF

SEQ ID NO: 31 - protein, 16 aa, Artificial Sequence
  Synthetic
DFSATWCGPCKMIKPF

SEQ ID NO: 32 - protein, 15 aa, Artificial Sequence
  Synthetic
FSATWCGPCKMIKPF

SEQ ID NO: 33 - protein, 19 aa, Artificial Sequence
  Synthetic
LVVVDFSATWCGPCKMIKP

SEQ ID NO: 34 - protein, 18 aa, Artificial Sequence
  Synthetic
VVVDFSATWCGPCKMIKP

SEQ ID NO: 35 - protein, 17 aa, Artificial Sequence
  Synthetic
VVDFSATWCGPCKMIKP

SEQ ID NO: 36 - protein, 16 aa, Artificial Sequence
  Synthetic
VDFSATWCGPCKMIKP

SEQ ID NO: 37 - protein, 15 aa, Artificial Sequence
  Synthetic
DFSATWCGPCKMIKP

SEQ ID NO: 38 - protein, 14 aa, Artificial Sequence
  Synthetic
FSATWCGPCKMIKP

(19) United States
(12) Patent Application Publication
(10) Pub. No.: US 2018/0221454 A1
(43) Pub. Date: Aug. 9, 2018

(54) THIOREDOXIN, THIOREDOXIN PEPTIDES OR DERIVATIVES FOR TREATMENT OF AGE-RELATED HYPERTENSION

(71) Applicant: BOARD OF REGENTS, THE UNIVERSITY OF TEXAS SYSTEM, AUSTIN, TX (US)

(72) Inventor: Kumuda Das, Tyler, TX (US)

(21) Appl. No.: 15/845,376

(22) Filed: Dec. 18, 2017

Related U.S. Application Data
(63) Continuation-in-part of application No. 15/323,545, filed on Jan. 3, 2017.
(60) Provisional application No. 62/020,146, filed on Jul. 2, 2014.
(60) Provisional application No. 62/435,395, filed on Dec. 16, 2016.

(51) Int. Cl. A61K 38/44; A61P 9/12; A61P 9/00

(52) U.S. Cl. CPC A61K 38/44; A61P 9/00; A61P 9/12; C12Y 108/04008

(57) ABSTRACT

The incidence of high blood pressure is strikingly high with advancing age, and is an independent prognostic factor for the onset or progression of a variety of cardiovascular disorders (CVD). There is a critical need for a curative therapy against age-related hypertension. Overexpression of human thioredoxin (Trx) a redox protein in mice prevented age-related hypertension. Chronic injection of recombinant human Trx (rhTrx) for 3-consecutive days reversed hypertension in aged wildtype mice that lasted for at least 20 days. Arteries of wildtype mice so injected or mice with Trx overexpression exhibited decreased arterial stiffness, greater endothelium-dependent relaxations, increased nitric oxide (NO) production and decreased superoxide anion (O₂.⁻) generation compared to appropriate controls. Collectively, a translational role of rhTrx in reversing age-related hypertension with long lasting efficacy is disclosed. Compositions and methods the treatment of cardiovascular disorders (CVD), primarily hypertension, comprise a therapeutically effective amount of thioredoxin-1 (Trx1 polypeptide, a pharmaceutically active Trx1 peptide fragment, or a functional derivative of Trx1 polypeptide or peptide, in a pharmaceutical carrier to ameliorate one or more symptom of the CVD.

### CROSS-REFERENCE TO RELATED APPLICATIONS

This application claims priority from U.S. Provisional application 62/435,395 filed 16 Dec. 2016, and is a continuation-in-part from U.S. Ser. No. 15/323,545 filed on 3 Jan. 2017, which was the US National Phase of PCT Application PCT/US205/037131 filed 23 Jun. 2015, and which claims priority from U.S. provisional application 62/020,146 filed 2 Jul. 2014.

### STATEMENT OF RIGHTS TO INVENTIONS MADE UNDER FEDERALLY SPONSORED RESEARCH

This invention was funded in part by grants from the National Heart Lung and Blood Institute, National Institutes of Health, HL107885, HL109397, and HL 132953 which provides to the United States government certain rights in this invention.

### FIELD OF THE INVENTION

The present invention relates in general to the field of cardiovascular disorders, and more particularly, hypertension and the treatment and reversal of age-related hypertension by chronically improving vascular redox and restoring eNOS function of high BP using thioredoxin and thioredoxin peptides, variants or derivatives.

### BACKGROUND OF THE INVENTION

Without limiting the scope of the invention, its background is described in connection with hypertension treatments. Hypertension is a common lifelong disease where arterial pressure is elevated to an undesired level. Globally, hypertension is very common in both developed and developing countries.

Aging is an independent risk factor for the onset of hypertension; in fact, people who are normotensive at 55 years of age have a 90% lifetime risk of eventually developing high blood pressure (Y. Higashi, et al. Hypertension Research: Official journal of the Japanese Society of Hypertension 35:1039-47 (2012)). Since hypertension is a major contributor to many cardiovascular diseases or disorders (CVD), including ventricular dysfunction, coronary artery disease and heart failure (D. Susic et al. Nat Clin Pract Cardiovasc Med 5:104-110 (2008)). The elderly are at a greater risk of developing cardiovascular diseases. Recent NIH systolic blood pressure (SBP) intervention trial (SPRINT) with 9361 hypertensive patients above the age of 50 showed that a more aggressive targeted SBP of 120 mmHg reduced the cardiovascular risk by 25-30% underscoring the severity and adverse impact of age-related hypertension (S. Oparil et al. Circulation 134:1308-10 (2016)). Current medical management of age-related hypertension includes the use of diuretics, renin-angiotensin system (RAS) antagonists, and calcium channel blockers (E. Pimenta et al. Nature Reviews. Cardiology 9:286-96 (2012)). Although this therapeutic strategy has reduced the incidence of cardiovascular diseases, complications arise in older patients, who often use many other drugs concurrently. A number of biochemical processes, for example, oxidative stress, reduced NO, high RAS activity and endothelial dysfunction have been identified as contributors to hypertension (E L Schiffrin, Hypertension 51:31-32 (2008); P M Vanhoutte et al., Acta Physiol (Oxf) 196; 193-222 (2009); J D Widder et al. Hypertension 54:338-44 (2009)); however, specific therapeutic interventions for multiple targets in age-related hypertension remain unavailable.

Although human studies have shown that large artery stiffness is a major cause of increased SBP with decreased diastolic blood pressure (DBP) (E G Lakatta, et al., Circulation 107:139-146 (2003) C Rammos et al., Mech Ageing Dev 135, 15-23 (2014)) in aging, temporal relationship between blood pressure and vascular stiffening remains unclear; specifically whether vascular stiffness precedes hypertension (B M Kaess et al., JAMA 308, 875-881 (2012)). Correlation of proximal aortic stiffness with the incidence of hypertension have been shown (Kaess et al., supra); higher carotid artery stiffness was also associated with incidence of hypertension (D. Liao et al. Hypertension 34:201-206 (1999)). However, a recent study showed that large artery and microvascular endothelial function jointly contribute to the development of clinical hypertension (Kaess et al., supra). Further, flow-mediated dilation (FMD) is a favorable indicator of endothelial function and decreased microvascular resistance, and which has been shown to be protective against development of hypertension (Kaess et al., supra). Studies in rodents have shown that dysfunctional endothelial nitric oxide synthase (eNOS) (Y M Yang et al. Am J Physiol Heart Circ Physiol 297:H1829-36 (2009); enhanced activity of xanthine oxidase (GP Dupont et al. J Clin Invest 89, 197-202 (1992); K. Nakazono et al., Proc Natl Acad Sci USA 88, 10045-48 (1991)), increased NADPH oxidase activity (B. Lassegue et al., Arterioscler Thromb Vasc Biol 30:653-61 (2010); B. Lassegue et al. Circ Res 110:1364-90 (2012)) and decreased antioxidant defense during the aging process are linked to dysfunction of the endothelium and consequent development of hypertension.

One of underlying mechanism associated with age-related vascular endothelial dysfunction is the chronic oxidation of vascular proteins that occurs over the life span of an individual resulting in the loss of arterial relaxation and consequent increase in blood pressure. Accumulating evidence suggests that bioavailability of NO, a critical endothelium-derived relaxing factor produced by eNOS, becomes impaired during aging resulting in increased vascular resistance and high blood pressure (CA Hamilton et al., Hypertension 37, 529-34 (2001); S Taddei et al., Hypertension 38, 274-79 (2001); B van der Loo et al., J Exper, Med. (2000); N Andrawis et al., J. Amer. Geriat Soc. 48:193-8 (2000); D R Seals et al., Clin Science 120:357-375 (2011); P E Gates et al., Exper Physiol. 94:311-16 (2009)). A dysfunctional eNOS produces superoxide (O₂.⁻) by transferring electrons to molecular oxygen (O₂) instead of L-arginine, resulting in the uncoupling of eNOS-L-arginine pathway of production of NO (MJ Crabtree et al. J Biol Chem 288, 561-69 (2013)). Further, O₂.⁻ produced by dysfunctional eNOS is also known to react with NO resulting production of vascular peroxynitrite (ONOO⁻), which is a powerful oxidizer, and could cause further oxidative damage to aging vessels. Since increased O₂.⁻ is produced in a chronic manner by oxidatively modified vascular proteins due to advancing age, an intervention that prevents age-related oxidation of these proteins is likely to protect the development of hypertension. Further, an agent that reverses the oxidatively modified proteins to their native state is expected to reverse age-related hypertension. In contrast, removal of O₂.⁻ due to increased expression of antioxidant enzymes or by injecting therapeutic levels of superoxide dismutase (Sod) mimetic is expected to remove O₂.⁻ produced by the dysfunctional eNOS or NADPH oxidase that may prevent further oxidation of vascular proteins, but these agents will not regenerate the dysfunctional eNOS to a functional state, nor can they reverse other oxidized proteins or enzymes to their native state.

Consistent with this reasoning, a study has shown that TEMPOL, a Sod1 mimetic had no effect on decreasing hypertension in heterozygote Sod1 knock out mice (Sod1^{+/-}) that express 50% less SOD and are hypertensive. However, TEMPOL was effective in decreasing endothelial dysfunction in these mice indicating that TEMPOL could compensate for the 50% loss of Sod1 in Sod1^{+/-} mice (SP Didion et al., Hypertension 48:1072-79 (2006)). Interestingly, this study showed that the activity of Sod1 did not change between young or aged mice, although the blood pressure was noted to be significantly higher. In contrast, another recent study has shown that antioxidative therapy with TEMPOL improved arterial stiffness and decreased endothelial dysfunction in mice, but it had no effect on blood pressure as this study did not find any increase in blood pressure of aged mice contrast to many other studies that have shown increased blood pressure in aged mice (Rammos et al., supra; BS Fleenor et al. Aging Cell 11:269-76 (2012)). Therefore, agents that could reverse oxidatively modified proteins to their native state may provide protection against age-related hypertension, whereas antioxidants alone may have limited effectiveness.

### Thioredoxin (Trx)

Thioredoxin (Trx) is a small (12 kDa) cytosolic redox protein that is an electron donor for ribonucleotide reductase (RNR) for the synthesis of deoxyribonucleotides, a rate-limiting step in DNA replication (H. Muniyappa et al. J Biol Chem 284:17069-81 (2009)). Trx also is an electron donor for peroxiredoxins (Prx) that detoxify peroxides (KC. Das et al. Am J Respir Cell Mol Biol 25:226-32. (2001)). Additionally, a major function of Trx is to regenerate —SH group enzymes and proteins, which are inactivated by oxidation (A. Holmgren et al., Meth Enzymol 252:199-208 (1995); A. Holmgren, et al., Biochem Soc Trans 33; 1375-77 (2005)). Trx scavenges hydroxyl radicals, quenches singlet oxygen and induces mitochondrial Sod2 (KC Das et al., Biochem Biophys Res Commun 277:443-47. (2000); K C Das et al., Am J Respir Cell Mol Biol 17:713-26. (1997)). Thus, Trx is not only a radical scavenger or inducer of Sod2, but also converts oxidized proteins to native proteins due to its disulfide reductase properties (A. Holmgren, Annu Rev Biochem 54:237-71 (1985)). The expression of Trx is decreased in spontaneously hypertensive rats (SHR) and in stroke-prone hypertensive rats

(M. Tanito et al., Antioxid Redox Signal 6:89-97 (2004)). However, it remains unclear whether increasing Trx levels would prevent hypertension. Another study showed that overexpression of mitochondrial Trx2 decreased angiotensin II (Ang II)-mediated increase in blood pressure in mice, demonstrating that reactive oxygen species (ROS) generated by angiotensin II in the mitochondria could be important in development of Ang II-related hypertension (Widder et al., supra). However, these studies did not address the role of Trx in age-related hypertension. Taken together, the role of Trx in age-related hypertension remains far from clear.

Since the homozygous Trx knockout mouse is embryonic lethal (M. Matsui et al. Dev Biol 178: 179-185 (1996)), it has been impossible to determine if Trx is required to maintain vascular redox homeostasis during age-related hypertension, or if it contributes to the regulation of hypertension. Since Trx is known to regenerate oxidatively modified proteins by its disulfide-thiol reductase properties, the present inventor and colleagues developed a transgenic (Tg) mouse strains that constitutively expresses higher levels of human Trx than non-transgenic (NT) mice, and another strain was designed to be deficient in reduced Trx by mutating its redox-active Cys32-Cys35 residues to serine (K. C. Das, Am J Physiol Lung Cell Mol Physiol 308:L429-442 (2015); R. H. Hilgers and K. C. Das, Hypertension 65:130-139 (2015); WO 2016/003702A1; US Pat Publ.),

Again, the incidence of high blood pressure with advancing age is strikingly high, and it is an independent prognostic factor for the onset or progression of a variety of CVDs. Although age-related hypertension is an established phenomenon, current treatments are only palliative, but not curative. Thus, there is a critical need for a curative therapy against age-related hypertension, which could greatly decrease the incidence of cardiovascular disorders.

These mice, double negative Trx transgenics (dnTrx-Tg) maintain only low levels of active Trx due to a dominant-negative effect on the endogenous protein. The studies leading to the present invention were directed to determining whether:
increased expression of Trx from the beginning of life would protect against development of age-related endothelial dysfunction and high blood pressure due to protection of critical vascular proteins against age-related oxidation;
high levels of recombinant human Trx (rhTrx) would be able to reverse an aged hypertensive phenotype to a normotensive one.

Citation of the documents above (and below) is not intended as an admission that any of these are pertinent prior art. All statements as to the date or representation as to the contents of these documents are based on the information available to the applicant and do not constitute any admission as to the correctness of the dates or contents of these documents.

### SUMMARY OF THE INVENTION

The present inventor discovered that overexpression of Trx in mice protects against endothelial dysfunction and the development of age-related hypertension and that age-related hypertension is reversed by the treatment of aged mice with rhTrx, demonstrating its therapeutic efficacy in hypertension.

The present invention is directed to a pharmaceutical composition for treating a cardiovascular disorder comprising:
(a) thioredoxin-1 (Trx1) protein (SEQ ID NO:1), preferably recombinant human Trx1 (rhTrx1);
(b) a therapeutically active peptide of (a);
(c) a therapeutically active conservative amino acid sequence variant of (a) or of (b)
(d) a therapeutically active functional derivative of (a) or (b); or
(e) a combination of any one or more of (a)-(d),

In the pharmaceutical composition, the peptide of (b) is preferably a peptide of the sequence SEQ ID NO:5 to SEQ ID NO:38). The pharmaceutical carrier is preferably one that is adapted for intramuscular, intraperitoneal, intravenous, or subcutaneous administration, most preferably for iv administration.

The cardiovascular above disorder is preferably a hypertensive disorder, most preferably, aging-related hypertension.

Also provided is a pharmaceutical composition for (i) inhibiting aging-induced loss of endothelial cell function, (ii) preserving endothelial nitric oxide synthase (eNOS) protein function or (iii) activating eNOS protein function in a subject, comprising
(a) Trx-1 protein (SEQ ID NO:1), preferably rhTrx1;
(b) a pharmaceutically active peptide of (a)
(c) a pharmaceutically active conservative amino acid conservative amino acid sequence variant of (a) or (b)
(d) a functional derivative of (a or (b)
(e) a Trx1 system upregulator, or
(f) a Trx1 system activator,
   in a pharmaceutical carrier.

The present invention is directed to a method for ameliorating one or more symptom of a cardiovascular disorder, preferably hypertension, in a subject in need thereof, comprising administering to the subject a therapeutically effective amount of the above pharmaceutical composition, thereby ameliorating one or more symptom of said cardiovascular disorder.

In the above method, the pharmaceutical carrier is preferably one that is adapted for intramuscular, intraperitoneal, intravenous, or subcutaneous administration, most preferably for iv administration. In this method, the pharmaceutical composition may be administered every 0.5, 1, 2, 3, 4, 5, 6, or more months.

Also provided is a method for treating a subject having a CVD, preferably hypertension, comprising administering to the subject a therapeutically effective amount of (a) a Trx1 system upregulator or activator, and/or (b) an agent that causes depletion of nitric oxide (NO) in the subject's body in a pharmaceutical carrier, preferably one that is adapted for intramuscular, intraperitoneal, intravenous, or subcutaneous administration, most preferably for iv administration.

The present invention is also directed to a method for (i) inhibiting aging-induced loss of endothelial cell function, (ii) preserving endothelial nitric oxide synthase (eNOS) protein function or (iii) activating eNOS protein function in a subject, comprising administering to the subject an effective amount of a pharmaceutical composition according to claim 6, thereby inhibiting the aging-induced loss of endothelial cell function or preserving or activating eNOS protein function. In one embodiment, this method is for preserving eNOS protein function in the subject; alternatively, the method is for activating eNOS protein function in the subject. The subject is preferably one who suffers from a CVD, more preferably, hypertension, most preferably, aging-related hypertension.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows that Trx-Tg mice have phenotypes that show decreased blood pressure. The basal blood pressure measurements show that Trx-Tg mice have lower blood pressure than the WT or mice expressing mutant Trx1.

FIG. 2 shows that Trx1 lowers angiotensin II mediated hypertension in mice.

FIG. 3 shows the entire spectrum of 14 day blood pressure measurement; at each time point the blood pressure of Trx-Tg mice is significantly lower.

FIG. 4 shows that the mesenteric 1 isolated from the Trx-Tg mice show significantly higher relaxation response compared to WT or mutant mice.

FIG. 5 is an image showing the redox state of vessels in WT, Trx-Tg and dnTrx-Tg in both young and old mice.

FIGS. 6A-6F show superior mesenteric artery (SAM) outward remodeling and depolarization- and agonist-induced contraction is maintained in Trx-Tg mice.

FIGS. 7A-7D show that aging-induced endothelial dysfunction is suppressed in Trx-Tg mice compared to WT and dnTrx-Tg mice.

FIGS. 8A-8D show preserved NO-mediated relaxing responses and endothelium-dependent hyperpolarization (EDH)-mediated responses in young and aged Trx-Tg mice, WT and dnTrx-Tg mice.

FIGS. 9A 9B show Trx enhances acetylcholine (Ach)-mediated NO release in SMA from young and aged mice.

FIGS. 10A-10F show aging-induced endothelial dysfunction is restored by exogenous dithiothreitol (DTT) and catalytically active h-Trx-1 in SMA from WT mice.

FIGS. 11A-11H show high levels of hTrx in transgenic mice outcompete age-related oxidation of vascular Trx and prevent the development of age-related hypertension: (A) Trx activity was assayed as described previously in carotid arteries (CA) derived from young (open bars) and aged (closed bars) NT (black), Trx-Tg (blue) and dnTrx-Tg (red) and expressed as nmoles of NADPH oxidized/mg protein/mg at 25° C. Values are represented as means±SEM (n=4-5); *P<0.05 versus NT young; ** P<0.05 versus NT young and Trx-Tg young; ## P<0.05 versus NT aged and Trx-Tg aged. (B) Trx activity in CA expressed as nmoles of 5-thio-2-nitro-benzene (TNB) formed per min/mg protein at 30° C. (C) Redox western blotting analysis revealing the redox state of Trx (Ox=oxidized; Red=reduced) in CA lysates from young and aged NT mice (upper panel) and young and aged Trx-Tg and dnTrx-Tg mice (lower panel). (D) Densitometry analysis of the ratio between Red and Ox Trx in CA lysates from young and aged Trx-Tg and dnTrx-Tg mice summarized in bar graphs. Values are represented as means±SEM of 4 repeated experiments each using CA lysates pooled from at least 3 mice. * P<0.05 versus Trx-Tg young; ** P<0.05 versus Trx-Tg young and aged; & P<0.05 versus Trx-Tg aged. (E) Expression of total Trx and Trx-2 in superior mesenteric arteries from young and aged NT, Trx-Tg and dnTrx-Tg mice. (F) Mean arterial pressure, (G) systolic blood pressure, and (H) diastolic blood pressure in mmHg measured in conscious and freely moving mice via radio-telemetry in young and aged NT (black bars), Trx-Tg (blue bars) and dnTrx-Tg (red bars). Values are expressed as means±SEM (N=6, NT and Trx-Tg; N=5, dnTrx-Tg). * P<0.05 versus young mice; ** P<0.05 versus NT aged and dnTrx-Tg aged mice.

FIGS. 12A-12B show that aging oxidizes vascular TRX in NT and dnTrx-Tg mice but not in Trx-Tg mice. Aortae from NT, Trx-Tg, and dnTrx-Tg young and aged mice were dissected out, cleaned of connective and adipose tissue, and homogenized in PBS (pooled from 3 mice of each strain). To label the reduced thiol groups of proteins in the homogenate, we incubated it with 100 μM AMS (4-acetamido-4′-maleimidylstilbene-2,2′-disulfonic Acid) for 8 hours at 40 C in dark. (A) Labeled homogenate was analyzed for reduced and oxidized TRX by Western blotting using anti-TRX antibodies. To detect total TRX, the homogenates was treated with 10 μM DTT and analyzed by Western blotting. The blot was reprobed with anti-GAPDH antibodies for normalization. (B) Relative densitometric units of reduced TRX band in FIG. 12A. Each bar shows the density of the corresponding reduced band from FIG. 12A (n=3; pooled mesenteric arteries of each genotype).

FIGS. 13A-13C. Mean arterial pressure (MAP) of aged Trx-Tg mice is lower than that of aged WT mice. Radio-telemetry recordings of MAP, heart rate, and activity for 24 hours in young or aged NT mice (A), young and aged Trx-Tg mice (B), and young or aged dnTrx-Tg mice (C). NT and Trx-Tg, n=6; dnTrx-Tg, n=5. MAP values (mean+/−SEM) are plotted at 30-min intervals.

FIGS. 14A-14B are graphs showing that peak systolic pressure is significantly decreased in Trx-Tg mice compared to WT or dnTrx-Tg mice. (A) The pressure wave recordings for NT young (black), Trx-Tg young (blue), dnTrx-Tg young mice (red). (B) Arterial blood pressure curves for aged NT, Trx-Tg, and dnTrx-Tg mice. Graphical

FIGS. 15A-15H. Pharmacological treatment of aged WT mice with rhTrx lowers high blood pressure in a sustained manner: (A) 24-hour continuous radio-telemetry recordings of mean arterial blood pressure (MAP), heart rate and activity in aged WT mice injected with rhTrx and followed for 1 day and 12 days after 3 dosages of rhTrx: Shaded region represents 12 h dark periods. n=6 per group, 20-28 months age. MAP and heart rate values are plotted at 30-min intervals. (B) After three days of recovery period, telemetric readings were collected and during the fifth day onwards mice were administered with three doses of recombinant hTrx (2.5 mg/kg) with 48 h interval between doses via tail vein. After the third dose of hTrx, telemetric readings were again collected for 24 h duration at starting at different time periods. Mean±SEM of mean arterial blood pressure (MAP) values are plotted at 24 h intervals over 21 days (n=6); (C) Averaged 12-hour day-time and night-time MAP values for each group shown as bar graph; (D) Systolic and (E) diastolic blood pressure of rhTrx injected WT mice; (F) Stiffness index was determined after 7-days and 18 days following the first rhTrx injection as mentioned in FIG. 2B; (G) levels of rhTrx in hearts of WT mice injected with rhTrx, (H) Redox state of rhTrx after 12 days post tail vein injection detected in blood plasma from aged WT mice. Oxidized (Ox) and reduced (Red) Trx was detected via native gel electrophoresis. * P<0.0001 vs. Saline (day), ** P<0.0001 vs. Saline (night)

FIGS. 16A-165 show high levels of Trx decrease superior mesenteric artery (SMA) vascular flow resistance: Representative high frequency ultrasound imaging of the SMA in mice. (A-F) Color Doppler is used for the identification of the SMA bifurcating from the aorta, whereas pulse Doppler wave signals reflect SMA blood velocity for young (A, C, and E) and aged (B, D, and F) NT (A and B), Trx-Tg (C and D) and dnTrx-Tg (E and F) mice. Flow velocity (mm/s) is plotted on the y-axis and time on the x-axis. Notice the difference in velocity scale between young and aged mice. Summarized values for peak systolic velocity (PSV) (G), end diastolic velocity (EDV) (H) and resistance index (I) for young (open bars) and aged (closed bars) NT (black), Trx-Tg (blue), and dnTrx-Tg (red) mice. Values are mean±SEM (n=5). * P<0.05 versus young mice; ** P<0.05 versus NT young and dnTrx-Tg young; # P<0.05 versus NT aged and dnTrx-Tg aged. Structural analysis of SMA from young and aged mice (J-P). Representative images of cannulated SMA from young and aged NT, Trx-Tg and dnTrx-Tg mice pressurized at 20, 60, 100 and 140 mmHg (J). Side branches are tied of with nylon threads. Pressure versus diameter relationship for cannulated and pressurized segments of SMA isolated from young (K) and aged (L) NT (black circles), Trx-Tg (blue upward triangles), and dnTrx-Tg (red downward triangles) mice. Pressure versus wall thickness relationships of SMA isolated from young (M) and aged (N) NT, Trx-Tg and dnTrx-Tg mice. Circumferential wall stress (CWS) versus incremental elastic modulus (E_{inc}) relationship for SMA isolated from young (O) and aged (P) NT, Trx-Tg, and dnTrx-Tg mice. Values are mean±SEM (n=11-17). * P<0.05 dnTrx-Tg young vs. Trx-Tg young; # P<0.05 NT aged and dnTrx-Tg aged versus Trx-Tg aged mice. Ages of mice used (in weeks) are shown next to graph I. Structural analysis of SMA from aged WT after tail vein injection of either saline or rhTrx (Q−S). Pressure versus diameter relationship (Q), pressure versus wall thickness (R) and CWS versus E_{inc} relationship (S) of isolated SMA from aged WT mice that underwent tail vein injection of saline (black circles) or rhTrx (2.5 mg/kg/day; blue circles). Values are mean±SEM (n=5-8). Arterial structural parameters were obtained using MYOVIEWII software from DMT-USA, Inc.

FIGS. 17A-17H. show aging-induced endothelial dysfunction is attenuated in SMA of Trx-Tg mice compared to NT or dnTrx-Tg mice (A-D) Endothelium-dependent relaxations to exogenously added ACh without the presence of any inhibitors. SMA derived from young (A) and aged (B) mice were contracted with a submaximal concentration of phenylephrine (PHE) before adding cumulative concentrations of ACh (0.01-10 μM). (C) Summarized sensitivity to ACh (expressed as the negative logarithmic value, pEC₅₀) for young (open bars) and aged (closed bars) mice. (D) Summarized maximal relaxing responses (Eₘₐₓ) to 10 μM ACh for young (open bars) and aged (closed bars) mice. Endothelium-independent relaxations to the NO donor sodium nitroprusside. SMA from young (E) and aged (F) mice were treated for 30 min with L-NAME (100 μM) and indomethacin (10 μM), contracted with a submaximal concentration of PHE, and assessed with cumulative concentrations of SNP (0.0001-10 μM). Preserved NO-mediated relaxing responses in aged Trx-Tg mice, but not in NT and dnTrx-Tg mice. SMA derived from young (G) and aged (H) mice were treated for 30 min with indomethacin (10 μM), TRAM-34 (10 μM) and UCL 1684 (10 μM). SMA was contracted with a submaximal concentration of PHE, before assessing relaxing responses to cumulative concentrations of ACh (0.01-10 μM). Values are means±SEM. Number of mice and their age are shown below bar graphs.*P<0.05 versus Young mice, ** P<0.05 versus aged Trx-Tg mice.

FIGS. 18A-18E show that losartan acutely reduces MAP but does not chronically maintain MAP. (A) After a three-day recovery period after surgery implanting the PA-C10 transmitter in the carotid artery, telemetric readings were collected and mice were treated with three doses of losartan (0.1 mg/100 μl) subcutaneously at 48-hour intervals. Telemetric readings were again collected for 24 hours 12 days after the first injection with losartan. Means±SEM of MAP values were plotted through the 12th day (n=4). (B-D) Structural analysis of SMA from aged NT after tail vein injection of either saline or rhTRX. Pressure versus diameter relationship (B), pressure versus wall thickness (C), and CWS versus Einc relationship (D) of isolated SMA from aged NT mice that underwent injection of saline (black circles) or losartan (green circles). Values are mean±SEM (n=4). Arterial structural parameters were obtained using MYOVIEWII software from DMT-USA, Inc. (E) Overall endothelium-dependent ACh-mediated relaxations in SMA derived from aged NT mice that underwent injection with losartan for the duration of 12 days. SMA was contracted with a submaximal concentration of PHE

FIGS. 19A-19B show depletion of TRX by siRNA decreases SMA relaxation. (A) Segments of SMA were treated with TRX siRNA or ntRNA and analyzed by Western blotting. (B) Segments of SMA were treated with TRX siRNA or ntRNA, then mounted on a wire myograph and subjected to analysis of endothelial-dependent relaxation.

FIGS. 20A-20J show that tail vein (iv) injection improves endothelial function and reduces superoxide anion release. Overall endothelium-dependent ACh-mediated relaxations (A) and NO-mediated relaxing responses (B) in SMA derived from aged NT mice that underwent tail vein injection of rhTrx (2.5 mg/kg/day) for the duration of 12 days. (C) NO-mediated endothelium-dependent ACh-mediated relaxations of SMA incubated in DMEM for 24 hours with either a cocktail of catalytically active and recyclable Trx (hrTrx+TR+NADPH), rhTrx+TR, rhTrx, or vehicle (control). SMA were contracted with a submaximal concentration of PHE before assessing relaxing responses to cumulative concentrations of ACh (0.01-10 μM). Values are means±SEM (n=3-6). * P<0.05 versus saline-injected or control. (D) The level of hTrx was detected in the arterial segments used in FIG. 5C by performing western analysis on SMA lysates. Strong hTrx was detected in the lysates only when all three reactants (hTrx, Trx reductase (TrxR) and NADPH) were present in the cultured SMA. (E) O₂.⁻ production in isolated thoracic artery segments (TAS) (3 mm) from saline treated WT mice was measured via EPR spectrometry using spin trap BMPO as described in the Methods; (F) TAS were isolated from rhTrx injected WT mice; (G) TAS segments treated with a cocktail of catalytically active and recyclable Trx ex vivo for 24 hours (H) TAS segments with EC denudation (I) TAS segments with EC denuded and with 100 nM angiotensin II added ex vivo. (J) Amount of O₂.⁻ generated was determined by calculating double integrated peak area of BMPO—OOH EPR signals using Xenon 1.1b.45 software (Bruker) and plotted as bar graph. *P<0.01 versus untreated control.

FIGS. 21A-21G show that eNOS is a major source of O₂.⁻ in aged NT mice, but both eNOS and NADPH oxidase contribute to O₂.⁻ generation in dnTrx-Tg mice. (A) Fluorescent images showing in situ detection of O₂.⁻ in thoracic aortae isolated from young and aged NT, Trx-Tg and dnTrx-Tg mice. Aortae were treated with or without 1 μM VAS 2870 or 100 μM L-NAME for 1 hour and incubated with DHE (10 μM) for an additional 3 hours. (B) Generated O₂.⁻ was measured by quantitating mean red fluorescence intensity localized in nucleus and represented by the bar graph.* P<0.05 level (ANOVA, Tukey's post test), **P<0.05 versus L-NAME, n=5 (C) O₂.⁻ production measured via EPR spectrometry of isolated aortic segments obtained from aged NT (upper) and young NT (lower); (D) NT aged after segments were treated for 30 min with either the non-selective Nox blocker VAS2870 (1 μM) (upper) or NT aged with the non-selective eNOS blocker L-NAME (100 μM) (lower); (E) Trx-Tg aged (upper) and young (lower); (F) aged dnTrx-Tg mice (upper) and young dnTrx-Tg mice (lower); (G) Amount of O₂.⁻ generated was determined by calculating double integrated peak area of BMPO—OOH EPR signals using Xenon 1.1b.45 software (Bruker) and plotted as bar graph. (n=5). *P<0.01 versus NT young; **P<0.01 versus NT aged (Y=Young; A=Aged).

FIGS. 22A-22G. Trx enhances ACh-mediated NO release in carotid arteries from young and aged Trx-Tg, but not dnTrx-Tg mice. (A) Fluorescence images of NO release by endothelium in longitudinally opened and en face carotid arteries is visualized by loading arteries with 5 μM DAF-FM and treating with 10 μM ACh for 5 seconds. In case of L-NAME, arteries were first incubated with 100 μM L-NAME for 30 min at 37° C., loaded with DAF-FM and treated with 10 μM Ach (n=5). (B) DAF-FM fluorescence upon NO release in the absence of ACh (black bars), presence of ACh (green bars) and ACh+L-NAME (red bars) was quantitated using AxioVision 4.9 software and bar graph represents mean±SEM of mean fluorescence intensity of five arterial segments. * P<0.01 versus NT Young control; ** or † P<0.01 versus NT and dnTrx-Tg Young+ACh, †† P<0.01 versus NT and dnTrx-Tg Aged+ACh. (C) SMA from young and aged NT, Trx-Tg and dnTrx-Tg mice were isolated and after indicated treatments, NO formation was detected by EPR spectroscopy using NO spin trap Fe-(MGD)₂ as described in Methods; (D). Double integrated peak area of NO—Fe (MGD)₂ EPR signals were quantitated and plotted as bar graph. *P<0.01 versus NT young; **, P<0.01 versus NT aged, n=4; (E) Pooled mesenteric arteries from young and aged mice (NT, Trx-Tg and dnTrx-Tg, n=3) were lysed and probed for phospho-eNOS^{Ser1177}, total eNOS and human Trx (F) Human coronary artery endothelial cells (HCAEC) were infected with control (Ad-LacZ) or human Trx overexpressing (Ad-Trx) adenovirus for 36 hours, then treated with PI₃ kinase inhibitor (LY294002; 10 μM) for 1 hour. Cell lysate was analyzed for phospho-eNOS^{Ser1177}, total eNOS, pAkt^{Ser473}, human Trx and β-actin by western analysis; (G) Representative blots of mesenteric artery protein lysates probed for phospho-eNOS^{Ser1177} and total eNOS from aged WT mice that were injected with 3 doses of rhTrx with 48 hour intervals rhTrx (2.5 mg/kg/day) or saline for the duration of 12 days. Blots from two saline and two rhTrx-injected mice are shown.

FIGS. 23A-23I. TRX does not preserve total BH₄ concentration, and sepiapterin does not reverse endothelial (renal) dysfunction. (A-C) SMAs derived from aged NT (A), aged Trx-Tg (B), and aged dnTrx-Tg (C) mice were incubated with vehicle (CON) for 60 min followed by contraction with a submaximal concentration of phenylephrine (PHE) before adding cumulative concentrations of ACh (0.01-10 μM). After washing, SMAs were incubated with sepiapterin (50 μM; green lines) for 60 min, contracted with PHE, and subjected to the same concentrations of ACh. Values are expressed as means±SEM (n=4-6). (D-F) DTT, but not TEMPOL, restores vascular relaxation in SMA from aged NT mice. SMA derived from aged NT (D), aged Trx-Tg (E), and aged dnTrx-Tg (F) mice were incubated with vehicle (CON) for 60 min followed by contraction with a submaximal concentration of phenylephrine (PHE) before adding cumulative concentrations of ACh (0.01-10 μM). After washing, SMAs were incubated with the SOD mimetic TEMPOL (1 mM; orange lines). (G-I) SMA derived from aged NT (D), aged Trx-Tg (E), and aged dnTrx-Tg (F) mice were incubated with vehicle (CON) for 60 min followed by contraction with a submaximal concentration of phenylephrine (PHE) before adding cumulative concentrations of ACh (0.01-10 μM). After washing, SMAs were incubated with disulfide reductase dithiothreitol (DTT; 1 μM; light blue lines) for 60 min, contracted with PHE, and subjected to the same concentrations of ACh. Values are expressed as means±SEM (n=4-6). * P=0.0154 versus controls (G).

FIGS. 24A-24E show that dithiothreitol (DTT), but not TEMPOL restore vascular relaxations in SMA from aged NT mice. (A) The amounts of BH4 in the aortae (pmol/mg protein, *P=0.0001; young vs aged of all strains). (B-E) TRX prevents eNOS S-glutathionylation and maintains NOX4 protein concentrations. (B) Aortae from NT and Trx-Tg young and aged mice were analyzed by Western blotting using anti-PrS-SG or anti-eNOS antibodies. (C) Oxidized glutathione (GSSG) was measured using the DTNB glutathione reductase (GR) recycling assay. GSSG was expressed as nmol/mg protein. (D) Mesenteric arteries from young and aged NT, Trx-Tg, and dnTrx-Tg mice were isolated and homogenized. To obtain sufficient amounts of protein, mesenteric arteries from at least three mice were pooled for each group. NOX4, SOD1, SOD2, HO-1, and TRX concentrations in the homogenate were analyzed by Western blotting using their specific antibodies. Blots were re-probed with antibody for β-actin for protein normalization. (E) Total RNA from aortae was isolated and analyzed for Nox4, HO-1, and Gapdh mRNA by RTPCR.

### DETAILED DESCRIPTION OF THE INVENTION

Abbreviations used in this document include: TRX or Trx or Trx1 (used interchangeably to refer to thioredoxin); SMA (superior mesenteric artery. PHEN (phenylephrine); DTT (dithiothreitol), WT (wild-type); NO (nitric oxide) NOS (nitric oxide synthase); eNOS (endothelial nitric oxide synthase); O₂.⁻ (superoxide anions); rhTRX (recombinant human TRX); BP (blood pressure); SBP (systolic BP); DBP diastolic BP)

While making and using various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as appears in the claims.

The present invention shows that overexpression of human thioredoxin (TRX), a redox protein, in mice prevents age-related hypertension.

Further, chronic injection of rhTRX for 3 consecutive days reversed hypertension in aged wild type mice, and this effect lasted for at least 20 days. Arteries of wild type mice injected with rhTRX or mice with TRX overexpression exhibited decreased arterial stiffness, greater endothelium-dependent relaxation, increased NO production, and decreased superoxide anion (O₂.⁻) generation compared to either saline-injected aged WT mice or mice with TRX deficiency. Thus TRX therapy (using the full length protein, or peptides or derivatives thereof, is expected to reverse age-related hypertension with long-lasting efficacy.

The present invention provides a protein composition that can be used to control BP. The overexpression of this protein in Tg mice lowered blood pressure and inhibited the induction of angiotensin II mediated hypertension. Tg mice, while treated with angiotensin II, had significantly higher relaxation of the mesenteric arteries in contrast to normally expressing wild-type mice. This natural protein or a biologically active peptide fragment thereof, a conservative amino acid substitution variant of the protein or peptide fragment, or a functional derivative of the foregoing can be used to control hypertension and pulmonary hypertension.

Basic texts disclosing general methods of molecular and cell biology, all of which are incorporated by reference, include: Sambrook, J. et al., Molecular Cloning: A Laboratory Manual, 3rd Edition, Cold Spring Harbor Press, NY, 2001; Green M R and Sambrook, J. eds. Molecular Cloning: A Laboratory Manual, 4th Ed, Cold Spring Harbor Press, NY, 2012; Ausubel, F M et al. Short Protocols in Molecular Biology, Vol. 1-2, 5th ed. Current Protocols, New York, (2002 or current edition); Albers, B. et al., Molecular Biology of the Cell, 2^{nd} Ed., Garland Publishing, Inc., New York, N.Y. (2007); Freshney, Culture of Animal Cells: A Manual of Basic Technique and Specialized Applications, 6th ed., Wiley-Blackwell Liss, New York (2010); Krebs, J E et al. Lewin's GENES XI, 11th ed., Jones & Bartlett Learning (2012); Watson, J D, et al., Molecular Biology of the Gene, 7th ed., Cold Spring Harbor Laboratory Press, 2013; Watson, J D et al., Recombinant DNA: Genes and Genomes—A Short Course, 3^{rd} Ed., Cold Spring Harbor Laboratory Press, 2007; Lodish, H et al., Molecular Cell Biology, 6th ed, W. H. Freeman, New York, N.Y. (2007); Primrose, S B et al. Principles of Gene Manipulation and Genomics, 7th Ed., Wiley-Blackwell (2006); Glover, D M, ed., DNA Cloning: A Practical Approach, vol. I-III, Oxford Univ. Press, 1987; Nicholl, D S, An Introduction to Genetic Engineering, 3rd Ed., Cambridge University Press (2008); http://www.amazon.com/Oligonucleotide-Synthesis-Methods-Applicaitons-Molecular/dp/1617374415/ref=sr_1_1?s=books&ie=UTF8&qid=1394124515&sr=1-1&keywords=Oligonucleotide+Syntesis Herdewijn, P, Oligonucleotide Synthesis: Methods and Applications, Human a Press (2010).

### Expression Vectors and Host Cells

For making the proteins or peptides of this invention, expression vectors are provided that comprise a nucleic acid sequence encoding TRX1 (preferably SEQ ID NO:4) and operably linked to at least one regulatory sequence, which includes a promoter that is expressible in a eukaryotic cell, preferably in a mammalian cells, more preferably in a human cell. Additional expression control sequences may be included.

The term “expression vector” or “expression cassette” as used herein refers to a nucleotide sequence which is capable of affecting expression of a protein coding sequence in a host compatible with such sequences. Expression cassettes include at least a promoter operably linked with the polypeptide coding sequence; and, optionally, with other sequences, e.g., transcription termination signals. Additional factors necessary or helpful in effecting expression may be included, e.g., enhancers.

“Operably linked” means that the coding sequence is linked to a regulatory sequence in a manner that allows expression of the coding sequence. Known regulatory sequences are selected to direct expression of the desired protein in an appropriate host cell. Accordingly, the term “regulatory sequence” includes promoters, enhancers and other expression control elements. Such regulatory sequences are described in, for example, Goeddel, Gene Expression Technology. Methods in Enzymology, vol. 185, Academic Press, San Diego, Calif. (1990)).

Thus, expression cassettes include plasmids, recombinant viruses, any form of a recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors may include replicons (e.g., RNA replicons), bacteriophages) to which fragments of DNA may be attached and become replicated. Vectors thus include, but are not limited to RNA, autonomous self-replicating circular or linear DNA or RNA, e.g., plasmids, viruses, and the like (U.S. Pat. No. 5,217,879), and include both the expression and nonexpression plasmids. Where a recombinant cell or culture is described as hosting an “expression vector” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

Those skilled in the art appreciate that the particular design of an expression vector of this invention depends on considerations such as the host cell to be transfected and/or the type of protein to be expressed. The present expression vectors comprise the full range of nucleic acid molecules encoding the various embodiments of the TRX1 polypeptide and its variants and functional derivatives (defined herein) including homologues, polypeptide fragments, amino acid substitution variants, preferably conservative amino acid substitution variants, addition variants, and deletion variants etc.

The present expression vectors may be used to transfect host cells (in vitro, ex vivo or in vivo) for expression of the DNA and production of the encoded proteins or peptides. It will be understood that a genetically modified cell expressing the TRX1 polypeptide may transiently express the exogenous DNA for a time sufficient for the vector and/or cell to be useful for its stated purpose.

Human Thioredoxin-1 (Trx1)

The human thioredoxin-1 (Trx1) protein has the amino acid sequence SEQ ID NO:1 found in the following Genbank database entries and shown below: AAF87085.1, AAH03377.1, AAH54866.1, AAA74596.1, AAG34699.1, CAA38410.1 and AFH41799.1; the depositors and the associated publication (if any) are shown in the database entries all of which are incorporated by reference in their entirety. The a.a. sequence shown in single letter code is

| | |
|---|---|
| MVKQIESKTA FQEALDAAGD KLVVVDFSAT WCGPCKMIKP FFHSLSEKYS | 50 |
| NVIFLEVDVD DCQDVASECE VKCMPTFQFF KKGQKVGEFS GANKEKLEAT | 100 |
| INELV | 105 |

The active site of Trx1 is at positions 31-35: WCGPC (SEQ ID NO:2) shown in italics in SEQ ID NO:1 above, or alternatively, at positions 32-35: CGPC (SEQ ID NO:3.

Preferred is recombinant human Trx1 (rhTrx1) which is available commercially or may be produced using recombinant DNA technology and methods well-known in the art.

The native DNA coding sequence for human Trx1 is shown GenBank entry AF548001.1 (M. J. Rieder et al., Direct Submission (21 Sep. 2002) Genome Sciences, University of Washington which shows the entire gene, the coding sequence comprises the following positions of the 15,100 bp long sequence:

(1578 to 1601, 6551 to 6655, 7134 to 7193, 13171 to 13236, and 13802 to 13864). The full coding sequence including the 3′ stop codon, is SEQ ID NO:4 shown below, along with the encoded amino acid residue in single letter code.

| | |
|---|---|
| atg gtg aag cag atc gag agc aaG act gct ttt cag gaa gcc ttg | 45 |
| M V K Q I E S K T A F Q E A L | 15 |
| gac gct gca ggt gat aaa ctt gta gta gtt gac ttc tca gcc acg | 90 |
| D A A G D K L V V V D F S A T | 30 |
| tgg tgt ggg cct tgc aaa atg atc aag cct ttc ttt cat tcc ctc | 135 |
| W C G P C K M I K P F F H S L | 45 |
| tct gaa aag tat tcc aac gtg ata ttc ctt gaa gta gat gtg gat | 180 |
| S E K Y S N V I F L E V D V D | 60 |
| gac tgt cag gat gtt gct tca gag tgt gaa gtc aaa tgc atg cca | 225 |
| D C Q D V A S E C E V K C M P | 75 |
| aca ttc cag ttt ttt aag aag gga caa aag gtg ggt gaa ttt tct | 270 |
| T F Q F F K K G Q K V G E F S | 90 |
| gga gcc aat aag gaa aag ctt gaa gcc acc att aat gaa tta gtc | 315 |
| G A N K E K L E A T I N E L V | 105 |
| taa | |
| *** | |

The rhTrx1 may be produced in any cell line that is transfected with the DNA coding sequence under control of constitutive or inducible promoters and appropriate additional regulatory sequences all of which are well known and commonly used in the art.

Preferred peptides with wild-type sequence for use in the present invention are those that possess the biological activity or activities of Trx1 and comprise at least the active site and between about 5 and about 10 residues on either side of the CGPC active site. “these are shown in Table 1, below

TABLE 1
Preferred human Trx1 peptide fragments

| | SEQ ID | | SEQ ID |
|---|---|---|---|
| Human Trx1 Peptide | NO: | Human Trx1 Peptide | NO: |
| LVVVDFSATWCGPCKMIKPFFHSL | 5 | LVVVDFSATWCGPCKMIKPFF | 21 |
| VVVDFSATWCGPCKMIKPFFHSL | 6 | VVVDFSATWCGPCKMIKPFF | 22 |
| VVDFSATWCGPCKMIKPFFHSL | 6 | VVDFSATWCGPCKMIKPFF | 23 |
| VDFSATWCGPCKMIKPFFHSL | 7 | VDFSATWCGPCKMIKPFF | 24 |
| DFSATWCGPCKMIKPFFHSL | 8 | DFSATWCGPCKMIKPFF | 25 |
| LVVVDFSATWCGPCKMIKPFFHS | 9 | FSATWCGPCKMIKPFF | 26 |
| VVVDFSATWCGPCKMIKPFFHS | 10 | LVVVDFSATWCGPCKMIKPF | 27 |
| VVDFSATWCGPCKMIKPFFHS | 11 | VVVDFSATWCGPCKMIKPF | 28 |
| VDFSATWCGPCKMIKPFFHS | 12 | VVDFSATWCGPCKMIKPF | 29 |
| DFSATWCGPCKMIKPFFHS | 13 | VDFSATWCGPCKMIKPF | 30 |
| FSATWCGPCKMIKPFFHS | 14 | DFSATWCGPCKMIKPF | 31 |
| LVVVDFSATWCGPCKMIKPFFH | 15 | FSATWCGPCKMIKPF | 32 |
| VVVDFSATWCGPCKMIKPFFH | 16 | LVVVDFSATWCGPCKMIKP | 33 |
| VVDFSATWCGPCKMIKPFFH | 17 | VVVDFSATWCGPCKMIKP | 34 |
| VDFSATWCGPCKMIKPFFH | 18 | VVDFSATWCGPCKMIKP | 35 |
| DFSATWCGPCKMIKPFFH | 19 | VDFSATWCGPCKMIKP | 36 |
| FSATWCGPCKMIKPFFH | 20 | DFSATWCGPCKMIKP | 37 |
| | | FSATWCGPCKMIKP | 38 |

These may be produced by recombinant or chemical synthetic methods.

Mutants and variants of the human Trx1 are also contemplated for use in the present invention, including substitution variants in which 1-6, preferably 1-5, more preferably 1-4, more preferably 1-3, more preferably 1-2 or a single amino acid substitution mutant/variant. Preferably such substitutions are conservative ones (as known in the art). Preferred sites for mutation/substitution are one or more of Cys62, Cys69 and Cys73 which are bolded and underlined in SEQ ID NO:1 shown above. It is well known in the art how to screen for biochemical or biological activity of Trx1, or binding to specific anto-Trx1 antibodies, such that additional mutants and variants can be identified that retain biological activity.

The coding sequence (or non-coding sequence) of the nucleic acids useful herein preferably are codon-optimized for the species in which they are to be expressed, most particularly, humans. Such codon-optimization is routine in the art.

Preferred nt sequence variants of SEQ ID NO:1, include fragments, sequence encoding substitution variants, preferably conservative amino acid substitution variants, and/or addition variants, which collectively are referred to as “functional derivatives.”

The preferred nucleic acid sequence variants of the present invention have the following degrees of sequence identity with SEQ ID NO:1: about 50%, about 55%, about 60%, about 65%, about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, and any range derivable therein, such as, for example, from about 70% to about 80%, and more preferably from about 81% to about 90%; or even more preferably, from about 91% to about 99% identity.

While a preferred vector comprising a transgene encodes a full length polypeptide, preferably Trx (SEQ ID NO:1, as indicated) the present invention is also directed to vectors that encode a biologically active fragment or a conservative amino acid substitution variant or an addition variant of human Trx1.

A biologically active fragment or variant is a “functional equivalent”—a term that is well understood in the art and is further defined in detail herein. The requisite biological activity of the fragment or variant, using any method disclosed herein or known in the art has the following activity relative to the wild-type native polypeptide of at least about: 20%, 30%, 40%, 50%, 60%, 65%, 70%, 75%, 80%, 85%, 90%, about 95%, 97%, 99%, and any range derivable therein, such as, for example, from about 70% to about 80%, and more preferably from about 81% to about 90%; or even more preferably, from about 91% to about 99%.

It should be appreciated that any variations in the coding sequences of the present nucleic acids and vectors that, as a result of the degeneracy of the genetic code, express a polypeptide of the same sequence, are included within the scope of this invention.

The amino acid sequence identity of the variants of the present invention are determined using standard methods, typically based on certain mathematical algorithms. In a preferred embodiment, the percent identity between two amino acid sequences is determined using the Needleman and Wunsch (J. Mol. Biol. 48:444-453 (1970) algorithm which has been incorporated into the GAP program in the GCG software package (available at WWW URL gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at the WWW web address gcg.com, using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of Meyers and Miller (CABIOS 4:11-17 (1989)) which has been incorporated into the ALIGN program (version 2.0), using a PAM120 weight residue table, a gap length penalty of 12 and a gap penalty of 4. The nucleotide and amino acid sequences of the present invention can further be used as a “query sequence” to perform a search against public databases, for example, to identify other family members or related sequences. Such searches can be performed using the NBLAST and XBLAST programs (Altschul et al. (1990) J. Mol. Biol. 215:403-10). BLAST nucleotide searches can be performed with the NBLAST program, score=100, wordlength=12 to obtain homologous nucleotide sequences. BLAST protein searches can be performed with the XBLAST program, score=50, wordlength=3 to obtain amino acid sequences homologous to the appropriate reference protein. To obtain gapped alignments for comparison purposes, Gapped BLAST can be utilized (Altschul et al. (1997) Nucleic Acids Res. 25:3389-3402). When utilizing BLAST and Gapped BLAST programs, the default parameters of the respective programs (e.g., XBLAST and NBLAST) can be used. See World Wide Web URL ncbi.nlm nih gov.

The preferred amino acid sequence variant has the following degrees of sequence identity with the native, full length human Trx1 (SEQ ID NO:1); about 50%, about 55%, about 60%, about 65%, about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, and any range derivable therein, such as, for example, from about 70% to about 80%, and more preferably from about 81% to about 90%; or even more preferably, from about 91% to about 99% identity.

Preferred substitutions variants of the proteins and peptides of this invention are conservative substitutions in which 1, 2, 3, 4, 5, 6, 7, 8, 9 or 10, amino acid residues have been substituted by different residue. Most preferably less than 10, more preferably less than 5, and most preferably 1 or 2 residues are substituted. For a detailed description of protein chemistry and structure, see Schultz G. E. et al., Principles of Protein Structure, Springer-Verlag, New York, 1979, and Creighton, T. E., Proteins: Structure and Molecular Properties, 2^{nd} ed., W.H. Freeman & Co., San Francisco, 1993, which are hereby incorporated by reference. Conservative substitutions and are defined herein as exchanges within one of the following groups:
1. Small aliphatic, nonpolar or slightly polar residues: e.g., Ala, Ser, Thr, Gly;
2. Polar, negatively charged residues and their amides: e.g., Asp, Asn, Glu, Gln;
3. Polar, positively charged residues: e.g., His, Arg, Lys;
4. Large aliphatic, nonpolar residues: Met, Leu, Ile, Val (Cys); and
5. Large aromatic residues: Phe, Tyr, Trp.
   Pro, because of its unusual geometry, tightly constrains the chain.

Preferred substitutions are those which do not produce radical changes in the characteristics of the polypeptide molecule. Most acceptable deletions and insertions (addition variants) according to the present invention are those which do not produce radical changes in the characteristics of the protein or peptide.

Even when it is difficult to predict the exact effect of a substitution in advance of doing so, one skilled in the art will appreciate that the effect can be evaluated by routine screening assays, preferably the biological and biochemical assays described herein. The activity of a cell lysate or purified polypeptide or peptide variant is screened in a suitable screening assay for the desired characteristic. For example, a change in the immunological characteristic of the polypeptide or peptide molecule is assayed by alterations in binding to a given antibody, and is measured by an immunoassay. Biological activity is screened in an appropriate bioassay, as described herein or known in the art. When appropriate, measurement of receptor-ligand binding is a way to screen a variant for its biochemical or functional properties. Modifications of polypeptide properties such as redox or thermal stability, hydrophobicity, susceptibility to proteolytic degradation or the tendency to aggregate with carriers or into multimers are assayed by methods well known to the ordinarily skilled artisan.

Certain commonly encountered “non-standard” amino acids well-known in the art can be substituted for standard amino acids. These include, for example, include β-alanine (β-Ala) and other ω-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); norleucine (Nle); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); homoarginine (hArg); N-acetyl lysine (AcLys); 2,4-diaminobutyric acid (Dbu); 2,4-diaminobutyric acid (Dab); p-aminophenylalanine (Phe(pNH₂)); N-methyl valine (MeVal); homocysteine (hCys), homophenylalanine (hPhe) and homoserine (hSer); hydroxyproline (Hyp), homoproline (hPro), N-methylated amino acids and peptoids (N-substituted glycines).

Covalent modifications of the polypeptides are included and may be introduced by reacting targeted amino acid residues with an organic derivatizing agent that is capable of reacting with selected side chains or terminal residues.

Such chemically modified and derivatized moieties may improve a polypeptide's or peptide's solubility, absorption, biological half life, and the like. These changes may eliminate or attenuate undesirable side effects of the polypeptides in vivo. Moieties capable of mediating such effects are disclosed, for example, in Gennaro, A R, Remington: The Science and Practice of Pharmacy, Lippincott Williams & Wilkins Publishers; 20 Ed, 2005 (or latest edition)

Any of a number of known recombinant methods are used to produce a DNA molecule encoding the polypeptide fragment or variant. For production of a variant, it is routine to introduce mutations into the coding sequence to generate desired amino acid sequence variants of the invention. Site-directed mutagenesis is a well-known technique for which protocols and reagents are commercially available (e.g., Zoller, M J et al., 1982, Nucl Acids Res 10:6487-6500; Adelman, J P et al., 1983, DNA 2:183-93). These mutations include simple deletions or insertions, systematic deletions, insertions or substitutions of clusters of bases or substitutions of single bases.

Host cells may also be transfected with one or more expression vectors that singly or in combination comprise DNA encoding at least a portion of the Trx1 polypeptide. If desired, the polypeptide can be isolated from medium or cell lysates using conventional techniques for purifying proteins and peptides, including ammonium sulfate precipitation, fractionation column chromatography (e.g., ion exchange, gel filtration, affinity chromatography, etc.) and/or electrophoresis (see generally, Meth Enzymol, 22:233-577 (1971)). Once purified, partially or to homogeneity, the recombinant polypeptides of the invention can be utilized in pharmaceutical compositions as described in more detail herein.

The term “isolated” as used herein, when referring to a molecule or composition, such as a polypeptide or a nucleic acid, means that the molecule or composition is separated from at least one other compound (protein, other nucleic acid, etc.) or from other contaminants with which it is natively associated or becomes associated during processing. An isolated composition can also be substantially pure. An isolated composition can be in a homogeneous state and can be dry or in aqueous solution. Purity and homogeneity can be determined, for example, using analytical chemical techniques such as polyacrylamide gel electrophoresis (PAGE) or high performance liquid chromatography (HPLC). Even where a protein has been isolated so as to appear as a homogenous or dominant band in a gel pattern, there are trace contaminants which co-purify with it.

Host cells transfected or transduced to express the TRX1 polypeptide or a variant, homologue or functional derivative thereof are within the scope of the invention. For example, the polypeptide may be expressed in yeast, or mammalian cells such as Chinese hamster ovary cells (CHO) or, preferably human cells. Suitable host cells are known to those skilled in the art. Expression in eukaryotic cells leads to partial or complete glycosylation and/or formation of relevant inter- or intra-chain disulfide bonds of the recombinant protein.

Often, in expression vectors, a nucleotide sequence encoding a proteolytic cleavage site is introduced at the junction of the reporter group and the target protein to enable their separation after to purification of the expressed protein. Proteolytic enzymes for such cleavage and their recognition sequences include Factor Xa, thrombin and enterokinase. Typical fusion expression vectors include pGEX (Amrad Corp., Melbourne, Australia), pMAL (New England Biolabs) and pRITS (Pharmacia, Piscataway, N.J.) which fuse glutathione S-transferase, maltose E binding protein, or protein A, respectively, to the target recombinant protein. Inducible expression vectors include pTrc (Amann et al., Gene 69:301-15, 1988) and pET 11d (Studier et al., Gene Expression Technology: Meth Enzymol 185:60-89, 1990).

### Vector Construction

Construction of suitable vectors comprising the desired coding and control sequences employs standard ligation and restriction techniques which are well understood in the art. Isolated plasmids, DNA sequences, or synthesized oligonucleotides are cleaved, tailored, and re-ligated in the form desired.

Nucleic acids can also be chemically synthesized using standard techniques, including solid-phase synthesis which, like peptide synthesis, has been fully automated with commercially available DNA synthesizers (Itakura U.S. Pat. Nos. 4,598,049, 4,401,796 and 4,373,071; Caruthers et al. U.S. Pat. No. 4,458,066.).

The DNA sequences which form the vectors of the invention or vectors using during production of the nucleic acids of the invention are available from a number of sources. Backbone vectors and control systems are generally found on available “host” vectors which are used for the bulk of the sequences in construction. For the pertinent coding sequence, initial construction may be, and usually is, a matter of retrieving the appropriate sequences from cDNA or genomic DNA libraries. However, once the sequence is disclosed it is possible to synthesize the entire gene sequence in vitro starting from the individual nucleotide derivatives. The entire gene sequence for genes of sizeable length, e.g., 500-1000 bp may be prepared by synthesizing individual overlapping complementary oligonucleotides and filling in single stranded nonoverlapping portions using DNA polymerase in the presence of the deoxyribonucleotide triphosphates. This approach has been used successfully. Synthetic oligonucleotides are prepared by either the phosphotriester method as described by references cited above or the phosphoramidite method (Beaucage, S L et al., Tet Lett 22:1859, 1981; Matteucci, M D et al., J Am Chem Soc 103:3185, 1981) using commercially available automated oligonucleotide synthesizers. Once the components of the desired vectors are thus available, they can be excised and ligated using standard restriction and ligation procedures. Site-specific DNA cleavage is performed by treating with the suitable restriction enzyme or enzymes under conditions which are conventional in the art, the particulars of which are specified by the manufacturer of commercially available restriction enzymes. See, e.g., New England Biolabs, Product Catalog; Meth Enzymol. 65:499-560, 1980.

Any of a number of methods are used to introduce mutations into the coding sequence to generate the variants of the invention. These mutations include simple deletions or insertions, systematic deletions, insertions or substitutions of clusters of bases or substitutions of single bases. For example, modifications of DNA sequences are created by site-directed mutagenesis, a well-known technique for which protocols and reagents are commercially available (Zoller, M J et al., Nucleic Acids Res 10:6487-500, 1982; Adelman, J P et al., DNA 2:183-193, 1983). Using conventional methods, transformants are selected based on the presence of a selectable marker such as an antibiotic resistance gene depending on the mode of plasmid construction.

### Promoters and Enhancers

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. Two sequences of a nucleic acid molecule, such as a promoter and a coding sequence, are “operably linked” when they are linked to each other in a manner which permits both sequences to be transcribed onto the same RNA transcript or permits an RNA transcript begun in one sequence to be extended into the second sequence. Thus, two sequences, such as a promoter sequence and a coding sequence of DNA or RNA are operably linked if transcription commencing in the promoter sequence will produce an RNA transcript of the operably linked coding sequence. In order to be “operably linked” it is not necessary that two sequences be immediately adjacent to one another in the linear sequence.

The promoter sequences of the present invention must be operable in mammalian cells and may be either eukaryotic or viral promoters. While, preferred promoters are described below, other useful promoters and regulatory elements are also discussed. Suitable promoters may be inducible, repressible or constitutive, most preferably constitutive and tissue- or cell type-specific. A “constitutive” promoter is one which is active under most conditions encountered in the cell's environmental and throughout development. An “inducible” promoter is one which is under environmental or developmental regulation. A “tissue specific” or cell type-specific promoter is active in certain tissues or cell types of an organism.

An example of a constitutive promoter is the viral promoter MSV-LTR, which is efficient and active in a variety of cell types, and, in contrast to most other promoters, has the same enhancing activity in arrested and growing cells. Other viral promoters include that present in the CMV-LTR (from cytomegalovirus) (Bashart, M. et al., Cell 41:521, 1985) or in the RSV-LTR (from Rous sarcoma virus) (Gorman, C. M., Proc. Natl. Acad. Sci. USA 79:6777, 1982). Also useful are the promoter of the mouse metallothionein I gene (Hamer, D, et al., J. Mol. Appl. Gen. 1:273-88, 1982; the TK promoter of Herpes virus (McKnight, S, Cell 31:355-65, 1982); the SV40 early promoter (Benoist, C., et al., Nature 290:304-10, 1981); and the yeast gal4 gene promoter (Johnston, S A et al., Proc. Natl. Acad. Sci. USA 79:6971-5, 1982); Silver, P A, et al., Proc. Natl. Acad. Sci. (USA) 81:5951-5, 1984)). Other illustrative descriptions of transcriptional factor association with promoter regions and the separate activation and DNA binding of transcription factors include: Keegan et al., Nature 231:699, 1986; Fields et al., Nature 340:245, 1989; Jones, Cell 61:9, 1990; Lewin, Cell 61:1161, 1990; Ptashne et al., Nature 346:329, 1990; Adams et al., Cell 72:306, 1993.

The promoter region may further include an octamer region which may also function as a tissue specific enhancer, by interacting with certain proteins found in the specific tissue. The enhancer domain of the DNA construct of the present invention is one which is specific for the target cells to be transfected or is highly activated by cellular factors of such target cells. Examples of vectors (plasmid or retrovirus) are disclosed in (Roy-Burman et al., U.S. Pat. No. 5,112,767). For a general discussion of enhancers and their actions in transcription, see, for example Lewin's Genes XI, supra. Retroviral enhancers (e.g., viral LTR) may be used and are preferably placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency.

Preferred promoters and enhancers for the present invention are example is the promoter of the gene for CREB-BP/EP300 inhibitor 1 (CRI1) (also known as EID-1) (Gordon, G J et al., Am. J. Pathol. 166:1827-40) (2005); Gordon, G J. et al., Clin Cancer Res 11:4406-14) (2005). CRH is a CREB-binding protein and potential oncogene that, in functional assays, antagonizes the action of pRb, p300, and CREB-binding protein (CBP) histone acetylase activity (Miyake S et al., Mol Cell Biol 2000, 20:8889-8902). CRH binds and sequesters wild-type unphosphorylated (active) pRb, but also acts at points downstream of pRb in differentiation and proliferation control pathways. Another preferred promoter and promoter/enhancer combination is the mesothelin gene promoter (Hassan R, et al. Clin Cancer Res 10:3937-3947 (2004). Sequences of the promoter are shown in Hucl, T et al., Cancer Res 67:9055-65 (2007)

### Pharmaceutical Compositions, Routes of Administration and Doses

Administration of the compositions of the present invention may be by parenteral, subcutaneous (sc), intravenous (iv), intramuscular, intraperitoneal, transdermal routes or by various intrapulmonary routes including inhalation, lung instillation or by intrapleural administration. Alternatively, or concurrently, administration of compounds or compositions in the present methods may be by the oral route. A most preferred route is iv administration

The dosage administered will be dependent upon the age, health, and weight of the recipient, kind of concurrent treatment, if any, frequency of treatment, and the nature of the effect desired.

The composition is administered in an amount effective to achieve its intended purpose. While individual needs vary, determination of optimal ranges of effective amounts of each component is within the skill of the art. Typical dosages comprise 0.01 to 500 mg/kg/body wt, though more preferred dosages are described for certain particular uses, above and below.

The therapeutic dosage administered is an amount which is therapeutically effective, as is known to or readily ascertainable by those skilled in the art. The dose is also dependent upon the age, health, and weight of the recipient, kind of concurrent treatment(s), if any, the frequency of treatment, and the nature of the effect desired.

In addition to a pharmacologically active protein or peptide molecule, the present pharmaceutical compositions/preparations preferably contain suitable pharmaceutically acceptable carriers comprising excipients and auxiliaries which facilitate processing of the active compounds into preparations which can be used pharmaceutically as is well known in the art. Suitable solutions for administration by injection or orally, may contain from about 0.01 to 99 percent, active compound(s) together with the excipient.

The pharmaceutical compositions of the present invention are manufactured in a manner which is itself known, for example, by means of conventional mixing, granulating, dissolving, or lyophilizing processes. Suitable excipients may include fillers binders, disintegrating agents, auxiliaries and stabilizers, all of which are known in the art. Suitable formulations for parenteral administration include aqueous solutions of the proteins in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions that may contain substances which increase the viscosity of the suspension.

The pharmaceutical formulation for systemic administration according to the invention may be formulated for enteral, parenteral administration or administration by inhalation and all of these types of formulation may be used simultaneously to achieve systemic administration of the active ingredient.

Other pharmaceutically acceptable carriers the present composition are liposomes, pharmaceutical compositions in which the active protein is contained either dispersed or variously present in corpuscles consisting of aqueous concentric layers adherent to lipidic layers. The active protein is preferably present in the aqueous layer and in the lipidic layer, inside or outside, or, in any event, in the non-homogeneous system generally known as a liposomic suspension.

The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature.

The methods of this invention may be used reduce blood pressure in a subject in need thereof. The protein or peptide or pharmaceutically acceptable salt thereof is preferably administered in the form of a pharmaceutical composition as described above.

Doses preferably include pharmaceutical dosage units comprising an effective amount of the therapeutic agent. Dosage unit form refers to physically discrete units suited as unitary dosages for a mammalian subject; each unit contains a predetermined quantity of active material calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects

By an effective amount is meant an amount sufficient to achieve a regional concentration or a steady state systemic concentration in vivo which results in a measurable reduction in any relevant parameter of disease, such as blood pressure.

The amount of active compound to be administered depends on the nucleic acid, peptide/polypeptide or small organic molecule that is selected, the state of the disease or condition, the route of administration, the health and weight of the recipient, the existence of other concurrent treatment, if any, the frequency of treatment, the nature of the effect desired, for example, inhibition of primary tumor growth or of metastasis or growth metastatic cell once they have spread, and the judgment of the skilled practitioner.

A preferred single dose, given once daily for treating a subject, preferably a mammal, more preferably human who his suffering from CVD, more preferably hypertension, is between about 0.1 mg/kg and about 250 mg/kg, preferably between about 1 mg/kg and about 10 mg/kg, for example, via iv administration. Such a dose can be administered daily for anywhere from about 3 days to one or more weeks. Chronic administration is also possible, though the dose may need to be adjusted downward. The foregoing ranges are, however, suggestive, as the number of variables in an individual treatment regime is large, and considerable excursions from these preferred values are expected.

For continuous administration, e.g., by a pump system such as an osmotic pump, a total dosage for a time course of about 1-2 weeks is preferably in the range of 1 mg/kg to 1 g/kg, preferably 20-300 mg/kg, more preferably 50-200 mg/kg. After such a continuous dosing regiment, the total concentration of the peptide is preferably in the range of about 0.5 to about 50 μM, preferably about 1 to about 10 μM.

Effective doses and optimal dose ranges may be determined in vitro or in vivo using methods well-known in the art, including method described herein.

There are several distinct advantages to a protein drug over traditional low molecular weight antihypertensive drug therapies. First, the drug could be taken every 3-6 months as an injection rather than daily as a pill. Second, because the new drug is a naturally occurring human protein, there should be fewer or no adverse side effects to treatment. The protein drug has antioxidant properties that may have additional benefits for the heart. The protein represents a safer and healthier alternative with fewer side effects than current hypertension drugs.

Having now generally described the invention, the same will be more readily understood through reference to the following examples which are provided by way of illustration, and are not intended to be limiting of the present invention, unless specified.

### EXAMPLES

### Example 1: Materials and Methods

Animals—Generation of Trx and dnTrx Transgenic Mice

The details of generation of Trx-Tg and dnTrx-Tg are described in K. C. Das, (2015, supra; WO 2016/003702A1; US Pat Publ. 2017/00136100A1) All of which are incorporated by reference ini their entirety. Experiments were conducted in young (2 to 6 months) and aged (20 to 28 months) NT, Trx-Tg and dnTrx-Tg mice. The background of these mice was C57BL/6J. For rhTrx injection experiments aged wildtype (WT) C57BL/6 mice were obtained from National Institutes of Aging and maintained at the animal facility of Texas Tech University, Lubbock. All procedures were approved by the Institutional Animal Care and Use Committee at the Texas Tech University Health Sciences Center and were consistent with the Guide for the Care and Use of Laboratory Animals published by the National Institute of Health.

Culture of Superior Mesenteric Arteries (SMA)

Segments of SMA from aged NT mice were placed in 100 μL DMEM solution containing rhTrx (1 μg/μL), rhTrx+TrxR (10 U), rhTrx+TrxR+NADPH (2 mM), or none. The four segments were placed in a CO₂ incubator at 37° C. for 20 hours. The next day, segments were mounted on the wire-myograph and assessed for NO-mediated relaxing responses as described below Immediately after the experiment the segments were snap-frozen in liquid nitrogen for further analysis.

Endothelium-Dependent Relaxation Responses

Segments of SMA from aged WT mice were placed in 100 mL DMEM solution containing h-Trx-1 (1 g/L), h-Trx-1+TrxR (10 U), h-Trx-1+TrxR+NADPH (2 mM), or none. The four segments were placed in a CO₂ incubator at 37° C. for 20 hours. The next day, segments were mounted on the wire-myograph and assessed for NO-mediated relaxing responses as described herein.

Endothelium-dependent relaxation responses to cumulative concentrations of acetylcholine (ACh; 0.01-10 μM) were determined in SMAs contracted with PHEN (3-10 μM). To study NO-mediated relaxation responses, SMAs were treated with the non-selective cyclooxygenase blocker indomethacin (INDO; 10 μM) to inhibit vasodilator prostanoids. In addition, SMAs were treated with the selective endothelial Ca2+-activated K+ channel blockers (TRAM-34; 1 μM) and (UCL) 1684 (1 μM) to inhibit IK1 and SK3 channel activity, respectively, and subsequent inhibition of the endothelium-dependent hyperpolarization (EDH) relaxation. All three inhibitors were incubated for 30 min prior contraction with PHEN. EDH relaxations were recorded with a combination of the non-selective NO synthase blocker N-ω-nitro-L-arginine methyl ester (L-NAME; 100 μM) and INDO (10 μM). Endothelium-independent relaxation responses to cumulative concentrations of the NO donor sodium nitroprusside (SNP; 0.1 nM-10 μM) were determined in SMA treated with L-NAME and INDO. In a subset of aged mice, SMAs were treated with DTT; 100 μM) for 30 min in the wire-myograph chamber before assessing ACh-mediated relaxing responses.

Ultrasound Imaging

Ultrasound data were acquired using a high-frequency high-resolution ultrasound (Vevo 3100, Visualsonics, Toronto, Canada) imaging platform equipped with a linear array transducer (MS 400×, frequency 18-38 MHz). The mouse was anesthetized with 1.5% isoflurane (delivered in 100% O₂). The mouse was then placed on a heated procedure board with isoflurane supplied through a nose cone. The limbs were gently taped to ECG electrodes coated with electrode cream and a rectal thermometer was inserted for maintenance of normothermia (37° C. internal temperature). Fur on the belly was carefully removed using a chemical hair remover cream (Nair, Church & Dwight Co, Inc; Princeton, N.J.), and warmed ultrasound gel was liberally applied to ensure optimal image quality. The gain was set to around 30 dB. The SMA was imaged in a short-axis B-mode view. Next, the SMA blood flow was visualized in Color Doppller Mode to identify the highest color intensity. Using Pulse-Wave Doppler Mode the velocity signals were visualized. Peak systolic velocity (PSV) and end diastolic velocity (EDV) were measured over 5 cardiac cycles using the VEVO Lab software. The resistance index (RI) was calculated as (PSV−EDV)/PSV.

Activity Assays for Trx and TrxR

Frozen carotid arteries were homogenized in 0.05 M potassium phosphate buffer (pH 7.0) containing 1 mM EDTA. Following homogenization in a Waring® blender, the homogenate was centrifuged in a microfuge at 14,000 rpm at 4.0° C. for 45 mins. The supernatant was transferred to another tube, and the Trx activity assay was performed immediately as described in the inventors' previous publications. (K. C. Das et al., Am J Physiol 276, L530-539 Year) Briefly, the reaction mixture was comprised of NADPH (200 μM) and porcine insulin (80 μM; Sigma) in 0.05 M potassium phosphate buffer (pH 7.0) containing EDTA (1 mM) in a total volume of 0.25 ml. The assay was standardized using E. coli Trx and rat thioredoxin reductase. The reaction was started by addition of rat thioredoxin reductase (0.1 μM). Trx activity was calculated as μmoles of NADPH oxidized per minute per mg protein at 25° C. Trx reductase activity was determined by the method as described by Holmgren et al., Methods Enzymol 252:199-208 (1995). Peroxiredoxin activity was determined by the rate of decrease of NADPH using hydrogen peroxide as substrate. All assays were performed in Beckman DU800 spectrometer with Peltier temperature control and using quartz cuvettes.

Determination of Redox State of Trx

Mesenteric arteries were excised from NT, Trx-Tg and dnTrx-Tg mice and cleaned of connective and adipose tissue. To generate sufficient protein to detect the oxidized or reduced endogenous murine Trx, mesenteric arteries from NT mice were pooled (6 from young and 6 from aged mice). Segments were homogenized in 0.5 mL of carboxymethylation buffer and the oxidized and reduced Trx levels were detected as per previous publications by the present inventor's group (Muniyappa, supra; KC Das et al. Am J Physiol 276, L530-539 57 (1999)).

Pressure Myography

A segment of the SMA(˜5 mm) was isolated and cannulated on glass micropipettes positioned in a pressure myograph chamber (model 110P; Danish MyoTechnology, Inc., Aarhus, Denmark) as described elsewhere (RH Hilgers et al., J Pharmacol Exp Ther 333:210-17 (2010)). The NO donor sodium nitroprusside (SNP; 10 μM) was added to allow complete relaxation of the smooth muscle. A pressure-diameter relationship was established by recording the lumen diameter and wall thickness using the MYOVIEW II software (Danish Myotechnology, Inc.) during step-wise (10 mmHg) increases in intraluminal pressures from 10 to 140 mmHg According to Laplace's Law, wall tension (T in mN/mm) depends upon transmural pressure (Pₜ in mN/mm², where 100 mmHg equals 13.33 mN/mm²) and the radius of the artery (r in mm): T=Pₜ·r. Circumferential wall stress (σ in mN/mm²) is related to wall thickness (wt in mm) and wall tension (T): σ=T/wt. The incremental elastic modulus (E_{inc} in mN/mm²) was calculated according to Bergel: E_{inc}=1.5·rₒ²·rᵢ·ΔP/(rO₂.⁻ rᵢ²)·Δrᵢ, where rₒ=outer radius, rᵢ=inner radius, and ΔP=10 mmHg or 1.333 mN/mm².

Wire Myography

Mice were euthanized by an overdose of isofluorane and the mesentery was removed and placed in cold KRB. From each mouse, 2 mm segments of the SMA were carefully dissected and mounted in a wire-myograph (model 620M; Danish Myotechnology, Aarhus, Denmark) for the recording of isometric force development. SMA were incubated for 30 min in KRB with continuous aeration with 95% O₂/5% CO₂ and maintained at 37° C. SMAs were passively stretched. In brief, vessels were distended stepwise, in 100 mm increments to their optimal lumen diameters for active tension development. The vessels were stretched to a passive wall tension of 90% of the internal circumference of that achieved when the vessels were exposed to a passive tension yielding a transmural pressure of 100 mmHg. At this passive wall tension segments were contracted with high K+KRB (60 mM KCl in KRB solution; replacing equimolar NaCl with KCl), thus generating a stable contraction that reached a plateau after 10-15 min. This active wall tension was set to a 100% contraction level. After a 30 min washout period, cumulative concentration-response curves (CRC) were performed to the vasoconstrictor PHEN; 0.01-30 μM), which causes al-adrenergic-mediated contractions. See Das, 2015 supra).

Measurement of Intracellular NO Release

To study NO release in intact arteries, SMA from WT, Trx-Tg and dnTrx-Tg mice were freshly isolated, cleaned and cut open along its longitudinal axis. High level of care was applied to retain intact endothelium. Then the opened SMA were loaded with 4-amino-5-methylamino-2′,7′-difluorofluorescein (DAF-FM; 5 μM) for 5 min at 37° C. in oxygenated KRB and then treated with ACh (10 μM) for 5 seconds. After treatment, SMAs were fixed for 5 min in 3% paraformaldehyde and washed with PBS once and mounted using anti-fade mounting medium with endothelium facing the glass coverslip. Intracellular green fluorescence generated due to NO release was observed using Zeiss Axio Imager Z2 upright fluorescent microscope via 20.times./0.8 NA objective.

Measurement of superoxide production by DHE Aortic sections (5 mm) were incubated with 1 μM VAS 2870 or 100 μM L-NAME for 1 hour at 37° C. in warm and oxygenated KRB. Then 10 μM DHE was added and incubation was continued for 3 hours. Cryosections (8-10 μm) were mounted with medium containing DAPI and observed via Zeiss Axio Imager Z2 upright fluorescent microscope via 40×/1.4 NA objective. The red fluorescence resulted due to DHE reacting to superoxide was measured using Adobe Photoshop version 13.0.1×64. To measure DHE fluorescence intensity localized in nuclei, a mask was generated in DAPI channel, which was used in red channel to quantitate the mean fluorescence intensity localized in nucleus.

Electron Paramagnetic Spectrometry (EPR) for Detection of O₂.⁻ and NO:

Basal superoxide level in aortae was measured by electron paramagnetic resonance (EPR) spectrometry using spin trap 5-tert-Butoxycarbonyl-5-methyl-1-pyrroline-N-oxide (BMPO, Alexis Biochemical) as described by us previously (J. Subramani et al. J Biol Chem. 291:23374-89(2016)). Superoxide generated by aortic sections was detected as BMPO—OOH adducts using Bruker EMX X-band spectrometer at room temperature. NO formation by isolated SMA was detected by EPR spectroscopy using NO spin trap Fe^{2±}-(N-methyl-D-glucamine dithiocarbamate)₂ (Fe-MGD). SMA was incubated in 50 μL of MEM containing 10 μM ACh, 0.1 mM sodium ascorbate and 2 mM Fe-MGD. NO generated by LCA was detected as paramagnetic NO—Fe²⁺-MGD₂ adduct using Bruker EMX X-band spectrometer at room temperature (V. Kundumani-Sridharan et al. J Biol Chem, 290:17505-17519 (2015). Double integration of peaks were performed using Xenon 1.1b.45 (Bruker) software.

Proteins: PAGE and Western Analysis

Mesenteric arteries were excised (from WT, Trx-Tg or dnTrx-Tg mice) and cleaned of connective and adipose tissue. To generate sufficient protein to detect the oxidized or reduced endogenous murine Trx, mesenteric arteries from WT mice were pooled (6 from young and 6 from aged mice). Segments were homogenized in 0.5 mL of carboxymethylation buffer and the oxidized and reduced Trx levels were detected. Protein extracts were prepared from SMAs pooled from six mice. Segments were homogenized in cold PBS buffer in a glass douncer. The homogenate was centrifuged at 15,000 g for 15 min at 4° C. Supernatant (cystolic fraction) was kept on ice. The remaining pellet was resuspended in cold lysis buffer [50 mM TRIS.HCl (pH 7.4), 150 mM NaCl, 5 mmol/L EDTA, 1% Triton X-100, 50 mM NaF, 10 mM sodium pyrophosphate, 25 mM-glycerophosphate, 1 mM PMSF, 1 μg/mL leupeptin, 1 μg/mL aprotinin, 1 μg/mL pepstatin, and 1 mM Na3VO4]. The homogenate was centrifuged at 15,000 g for 20 min at 4° C. The supernatant (membrane fraction) was kept on ice. Protein concentration was determined with the BCA protein assay kit (Pierce Chemical, Rockford, Ill., USA). Low-temperature PAGE (LT-PAGE) was performed for detection of eNOS dimers. Briefly, running buffer and 6% gels were cooled on ice prior to loading of 15 g of protein from either the membrane fraction or the cytosolic fraction. The buffer tank was placed on ice during electrophoresis to maintain the temperature of the gel below 15° C. The running buffer was changed every 30 min with ice-cold running buffer. After electrophoresis the gels were transferred onto nitrocellulose membrane, and membranes were blocked by treatment with 5% non-fat dry milk in Tris-buffered saline containing 0.05% Tween-20 (TBS-T), followed by incubation with primary antibody (anti-eNOS; BD Translab, cat #610296) overnight at 4° C. After incubation with secondary antibodies, signals were detected with chemiluminescence autoradiography.

Chemicals/Reagents

The ionic composition of Krebs-Ringer Buffer (KRB) solution was as follows (mM): 118.5 NaCl, 4.7 KCl, 2.5 CaCl2, 1.2 Mg₂SO4, 1.2 KH₂PO4, 25.0 NaHCO₃, and 5.5 D-glucose. Anti-eNOS (cat #610297) was obtained from BD Transduction Laboratories, USA. DAF-FM) diacetate (cat # D23844) and Attachment Factor (cat # S006100) were purchased from Life Technologies, Grand Island, N.Y. The MV bullet kit (cat # CC-3202) was obtained from Lonza, Walkersville, Md. All chemicals were purchased from Sigma-Aldrich.

### Statistical Analysis

Results are shown as mean±SEM. Concentration-response curves were analyzed with two-way analysis of variance (ANOVA) followed by Bonferroni post-hoc test or Tukey's test for multiple comparisons. Other values were analyzed by paired and unpaired Student's t test. P<0.05 was considered to be statistically significant.

### Example 2

### Trx-Tg Mice have Lower BP than the Wildtype or Mice Expressing Mutant Thioredoxin

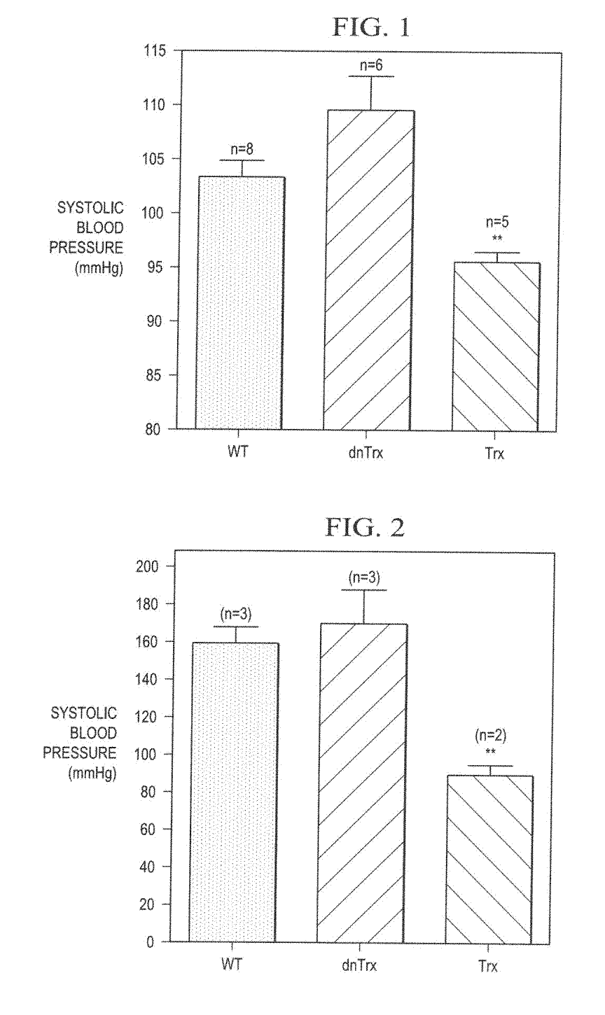

FIG. 1 shows Trx-Tg mice are phenotypes that show decreased BP. The basal BP measurements show that Trx-Tg mica have lower BP than the wildtype or mice expressing mutant thioredoxin.

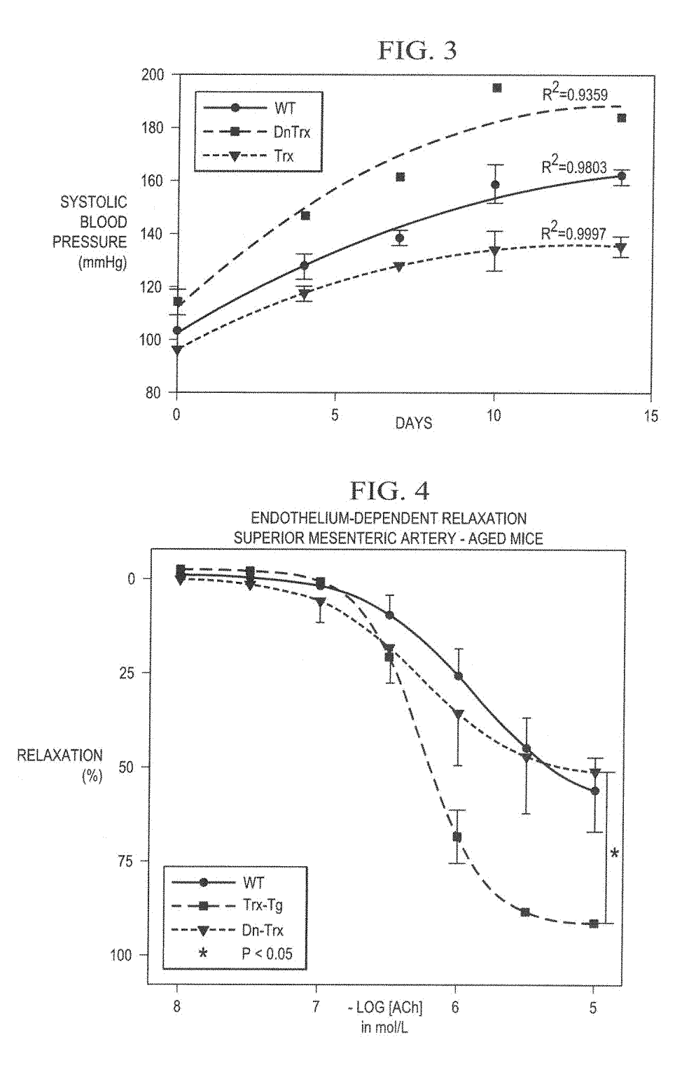

FIG. 2 shows thioredoxin lowers angiotensin II mediated hypertension in mice. Mice were infused with angiotensin II using osmotic minipumps for 14 days and the BP was measured in time intervals. As shown in FIG. 2, the BP measurement at the end of 10 days shows significant reduction in Trx-Tg mice compared to either wildtype or mutant mice. Additionally, the mutant mice show higher BP compared to controls.

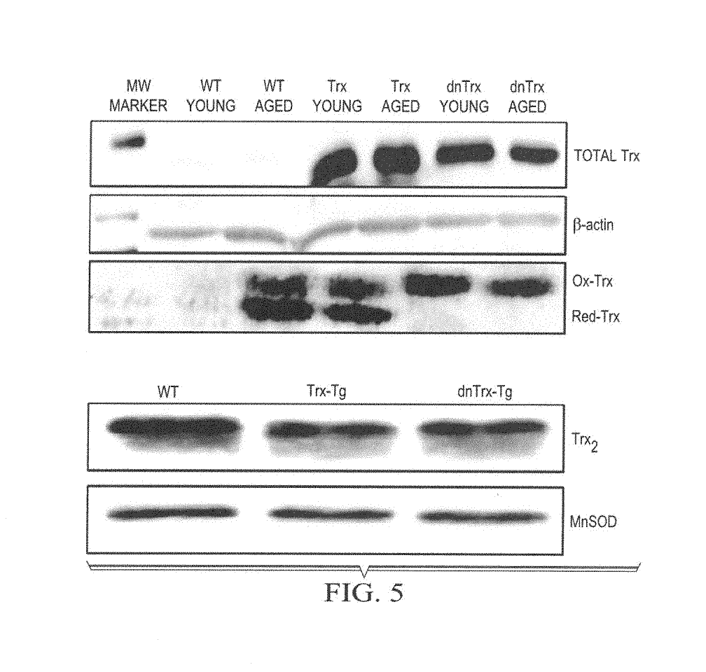

FIG. 3 shows the entire spectrum of 14 day BP measurement that shows that in each time point the BP of Trx-Tg mice is significantly lower.

### Example 3

### Vasculature of Trx Tg Mice

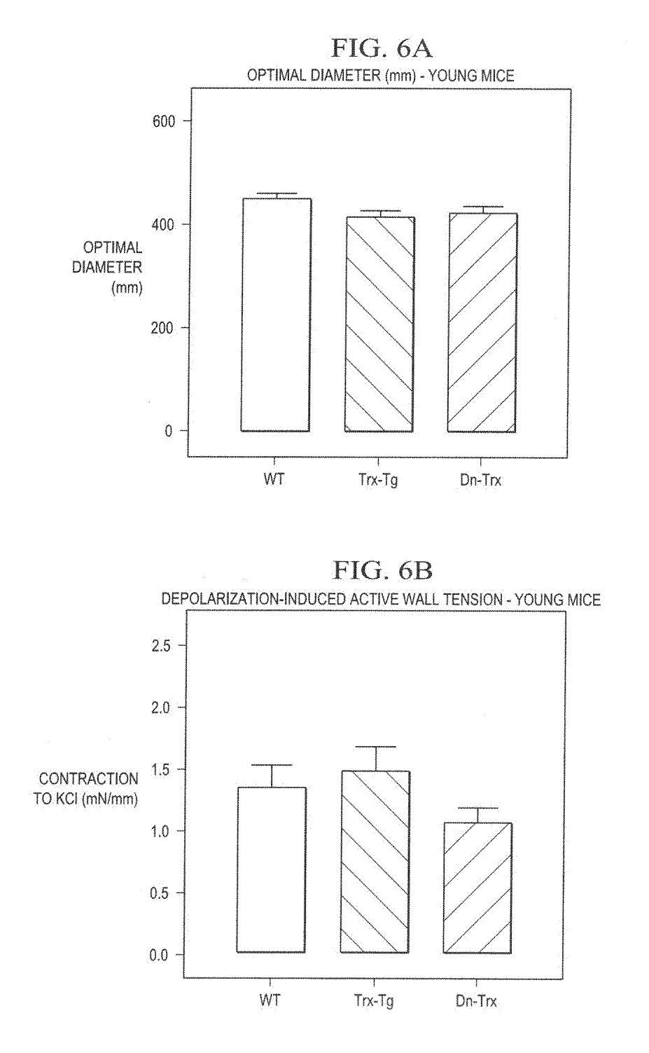

FIG. 4 shows the mesenteric artery isolated from the Trx-Tg mice show significantly higher relaxation response compared to wildtype or mutant mice. There is a significantly higher relaxation of mesenteric arteries isolated from Trx-Tg mice in response to Acetylcholine (Ach) compared to wildtype or mutant mice.

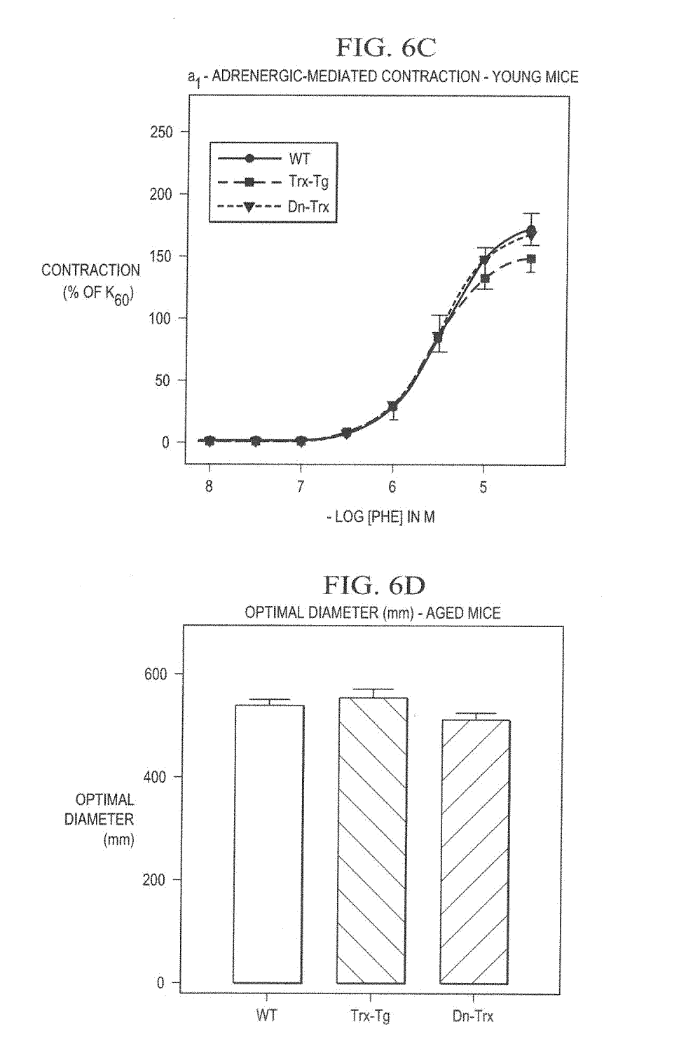

FIG. 5 is an image showing the redox state of vessels in WT, Trx-Tg and dnTrx-Tg in both young and old mice. The present data shows that aged Trx-Tg mice are normotensive, whereas aged dnTrx-Tg mice are more hypertensive compared to aged wildtype (wt) mice.

Additionally, Trx-Tg mice maintain significantly lower BP compared to aged wt or dnTrx-Tg mice. Increased levels of vascular Trx prevents age-related Trx oxidation observed in the vessels of Trx-deficient mice. Additionally, increased Trx levels maintained eNOS function in aged vessels in Trx-Tg mice, but not in dnTrx-Tg mice. The EC dysfunction observed in aged WT mice could be reversed by DTT, a chemical reductant. In contrast, DTT could not reverse EC dysfunction in aged vessels from dnTrx-Tg mice. Taken together, the cumulative oxidation of redox proteins in aging could be reversed by pharmacological intervention with Trx resulting in control of hypertension during aging.

FIG. 5 is an image showing the redox state of vessels in Wt, Trx-Tg and dnTrx-Tg in both young and old mice. The present data shows that aged Trx-Tg mice are normotensive, whereas aged dnTrx-Tg mice are more hypertensive compared to aged wildtype (WT) mice. Superior mesenteric arterial outward remodeling is enhanced with aging in Trx-Tg mice. The optimal diameters of segments of the main superior mesenteric artery (SMA) measured in the wire-myograph were comparable in young mice groups. The average diameter was 452±8 mm in WT, 425±11 mm in Trx-Tg, and 422±12 mm in dnTrx-Tg mice. Aging resulted in a statistically significant increase in lumen diameter in all the three mice groups compared to their younger counterparts. Interestingly, this outward remodeling was significantly larger in aged Trx-Tg (564±11 mm), compared to aged dnTrx-Tg mice 516±12 mm), but not to aged WT mice (543±14 mm).

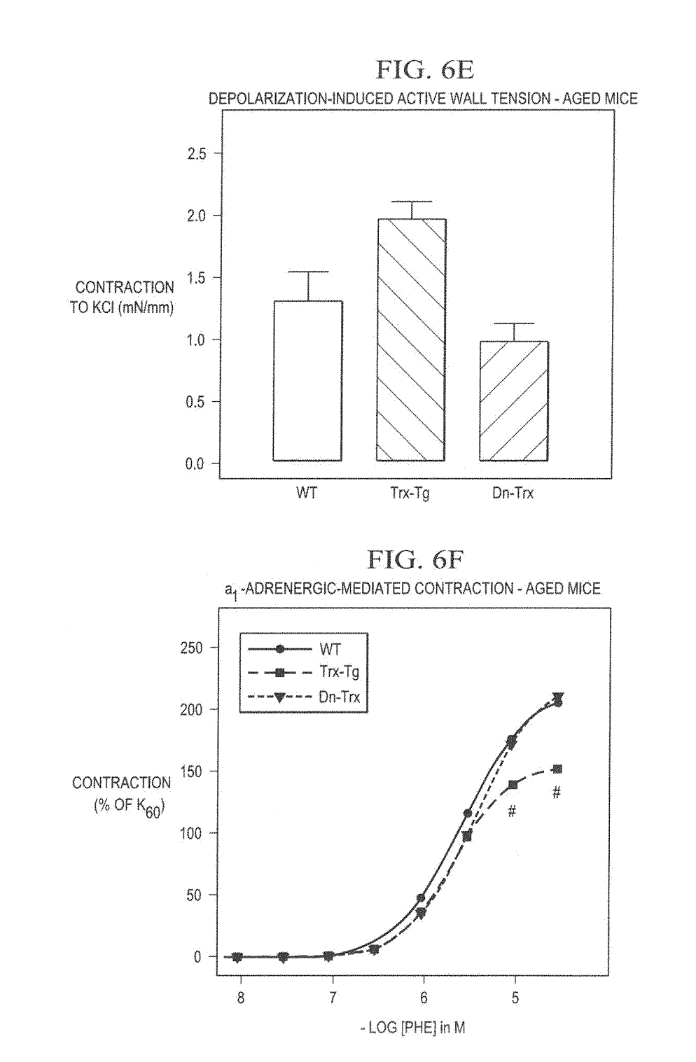

FIGS. 6A-6F show the SMA outward remodeling and depolarization- and agonist-induced contraction is maintained in Trx-Tg mice. FIGS. 6A-6D show optimal diameters, FIGS. 6B-6E show depolarization (60 mmol/L K+KRB)-induced and FIGS. 6C-6F show phenylephrine-induced contraction for SMA derived from young (upper panel) and aged (lower panel) wild-type (white bars), Trx-Tg (blue bars) and dnTrx-Tg (red bars) mice. Values are means±SEM (n=8-10 mice). * P<0.05 compared with Trx-Tg. Addition of 60 mM K+KRB resulted in a contraction due to opening of voltage-operated Ca2+ channels and subsequent transient contraction that reached a plateau after 10 to 15 min. Tensions did not differ between SMA derived from young mice. However, depolarization-induced contractions in SMA derived from aged Trx-Tg were statistically significantly larger compared to their WT and dnTrx-Tg counterparts. Phenylephrine (PHE) contracted SMA from young and aged mice in a concentration-dependent manner (FIGS. 6C and 6F). The sensitivity (pEC₅₀) to PHE did not differ significantly between arteries from young WT, Trx-Tg, and dnTrx-Tg mice (5.46±0.08, 5.56±0.12 and 5.48±0.07, respectively, FIG. 6E). The maximal tension (in mN/mm) generated by 30 mmolar PHE was also similar between arteries from young WT, Trx-Tg, and dnTrx-Tg mice (4.45±0.15, 4.05±0.19, and 4.04±0.12; respectively; FIG. 6C). Aging did not result in significant changes in pEC₅₀ values in the three mice groups (FIG. 6F), but maximal tension was statistically significantly increased in SMA from aged Trx-Tg mice (5.17±0.20) compared to their younger counterparts (FIG. 6F). In SMA from aged WT (4.87±0.34) and aged dnTrx-Tg (3.90±0.16) mice there was no statistically significant change in tension compared to their younger counterparts.

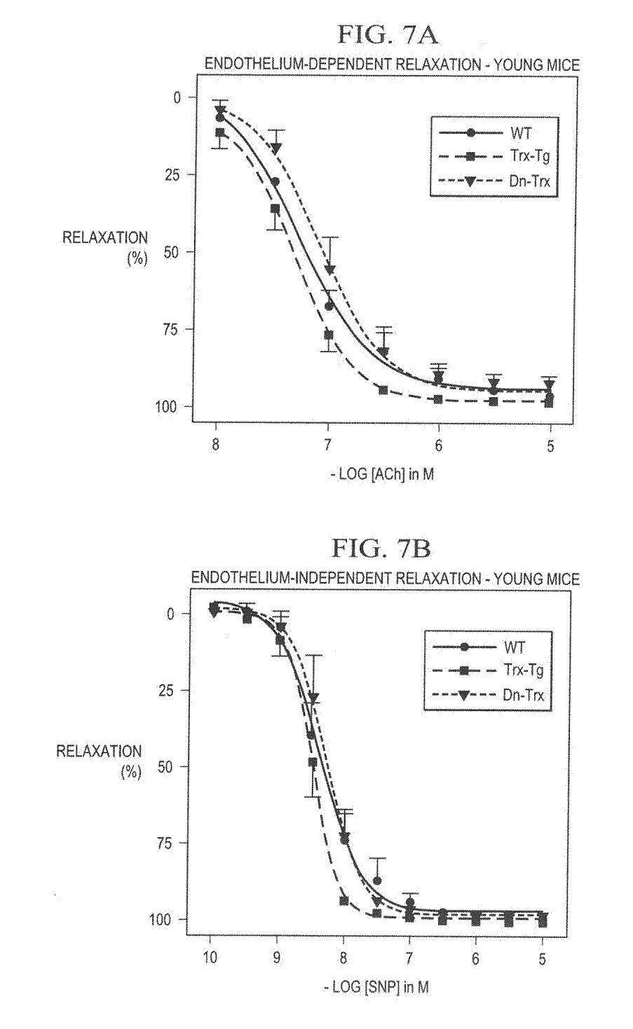

FIGS. 7A-7D show aging-induced endothelial dysfunction is suppressed in Trx-Tg mice compared to WT and dnTrx-Tg mice. FIGS. 7A-7D show aging-induced endothelial dysfunction is suppressed in Trx-Tg mice compared to WT and dnTrx-Tg mice. FIGS. 7A and 7C shows endothelium-dependent relaxations for SMA derived from young (FIG. 7A) and aged (FIG. 7B) wildtype (black circles), Trx-Tg (blue upward triangles) and dnTrx-Tg (red downward triangles) mice were contracted with a submaximal concentration of PHE before assessing relaxing responses to cumulative concentrations of ACh (0.01-10 mM). Endothelium-dependent relaxation was severely blunted with aging in DnTrx-Tg mice, but preserved in Trx-Tg mice. Endothelium-dependent acetylcholine (ACh)-mediated relaxations were reduced in SMA derived from young dnTrx-Tg mice compared to Trx-Tg mice (FIG. 7A). Sensitivity to ACh was statistically significantly decreased in SMA from dnTrx-Tg compared to Trx-Tg mice (6.88±0.13 versus 7.32±0.08), whereas sensitivity to ACh in SMA from young WT mice (7.02±0.11) was not different from either Trx-Tg or dnTrx-Tg mice. Maximal relaxation (Eₘₐₓ) in response to 10 μM ACh averaged 94±2%, 98±1%, and 92±3% in young WT, Trx-Tg, and dnTrx-Tg mice, respectively, which was comparable for all three groups. Aging resulted in a statistically significant right-ward shift in the concentration-response curves to ACh in all three mice groups (FIG. 7B). However, this shift was lower in Trx-Tg mice compared to WT and dnTrx-Tg mice, as evidenced by a statistically significantly greater pEC₅₀ value in aged Trx-Tg mice (6.74±0.06) compared to WT mice (6.35±0.09). Sensitivity for ACh in SMA from aged dnTrx-Tg mice could not be determined, since relaxations did not reach more than 50% in most SMA. Strikingly, Eₘₐₓ values to ACh were not different in SMA between young and aged Trx-Tg mice (98±1% versus 94±2%). This Emax for aged Trx-Tg mice was statistically significantly higher compared to aged dnTrx-Tg mice (47±11%), but not to aged WT mice (85±2%; FIG. 7B). FIG. 7C summarizes the relaxing responses from young and aged mice by depicted the calculated “area under the curve” values. The aging-induced changes were specific to the endothelium, since relaxing responses to the endothelium-independent NO donor sodium nitroprusside were comparable in SMA from both young (FIG. 7D) and aged (FIG. 7E) mice of all three groups.

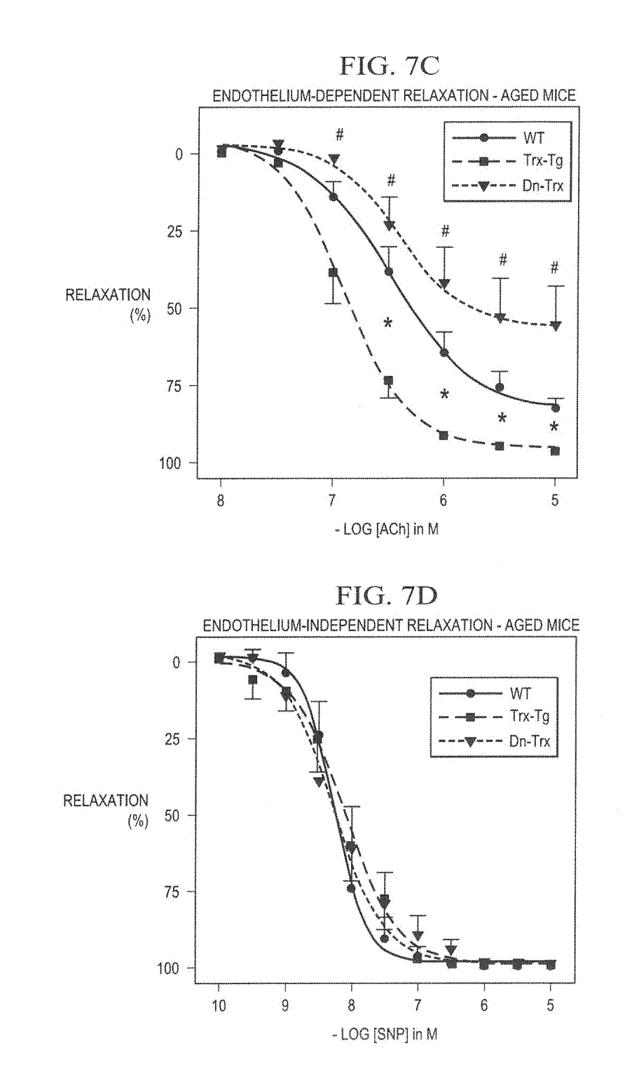

FIGS. 8A-8D show preserved NO-mediated relaxing responses and EDH-mediated responses in young and aged Trx-Tg mice, WT and dnTrx-Tg mice. NO-mediated endothelium-dependent relaxations in SMA derived from young (FIG. 8A) and aged (FIG. 8B) wildtype (black circles), Trx-Tg (blue upward triangles) and dnTrx-Tg (red downward triangles) mice were treated for 30 min with INDO (10 μM), and the combined presence of TRAM-34 (10 μM) and UCL 1684 (10 μM) to block vasodilator prostanoid release and EDH-mediated responses, respectively. SMA were contracted with a submaximal concentration of PHE, before assessing relaxing responses to cumulative concentrations of ACh (0.01-10 μM). FIGS. 8D and 8E show EDH-mediated responses in SMA derived from young (FIG. 8D) and aged (FIG. 8E) wildtype (black circles), Trx-Tg (blue upward triangles) and dnTrx-Tg (red downward triangles) mice were treated with L-NAME (100 mM) and INDO (10 mM) for 30 min, contracted with a submaximal concentration of PHE, and assessed with cumulative concentrations of ACh (0.01-10 μM). Values are means±SEM (n=6-8 mice). * P<0.05 versus Trx-Tg, # P<0.05 dnTrx-Tg versus WT and Trx-Tg, ** P<0.05 versus young mice. Since NO is the major endothelium-derived vasorelaxing factor in the superior mesenteric artery, we addressed the contribution of NO in ACh-mediated relaxations. To rule out vasorelaxing factors derived from cyclooxygenases, SMA were continuously treated with the non-selective cyclooxygenase inhibitor indomethacin (INDO; 10 μM). In addition, SMA were incubated with TRAM-34 (1 μM) and UCL 1684 (1 μM) to inhibit both IK1 and SK3 endothelial calcium-activated potassium channels, respectively to prevent endothelium-dependent hyperpolarization (EDH). Comparable NO-mediated relaxations were observed in SMAs from young mice (FIG. 8A). In SMA of Trx-Tg mice, NO-mediated relaxations were unaffected by advanced aging (FIG. 8B). However, WT and dnTrx-Tg mice showed markedly reduced NO-mediated relaxations with aging (FIGS. 8A and 8C). Next, we assessed EDH relaxations in the presence of L-NAME (100 μM) and INDO (10 μM). In young mice, comparable EDH responses were observed (FIG. 8B). Aging markedly blunted these EDH responses in SMAs from all three mice groups, but to a lesser extent in Trx-Tg mice. Based on the area under the curve values it is clear that NO is the major source of vasorelaxing factor in SMA.

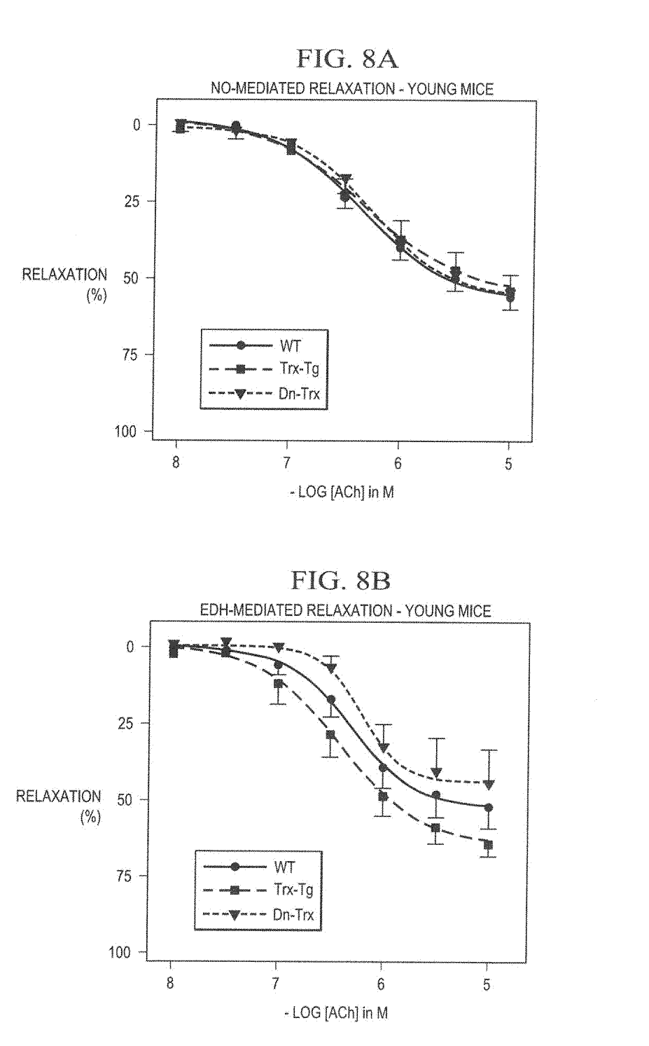

FIGS. 9A and 9B show Trx enhances ACh-mediated NO release in SMA from young and aged mice. FIG. 9A are fluorescence images of NO release by endothelium in longitudinally opened and en face arteries is visualized by loading arteries with 5 μM DAF-FM and treating with 10 μM ACh for 5 seconds. Images were captured using Zeiss Axio Imager Z2 upright fluorescent microscope via 20.times./0.8 NA objective. In case of 1-NAME, arteries were first incubated with 100 μM 1-NAME for 30 min at 37° C., loaded with DAF-FM and treated with 10 μM ACh. All incubations and treatments were carried out in oxygenated KRB. FIG. 9B shows DAF-FM fluorescence upon NO release in the absence of ACh (black bars), presence of ACh (green bars) and ACh+1-NAME (red bars) was quantitated using AxioVision 4.9 software and bar graph represents mean±SD of mean fluorescence intensity of five arterial segments. * P<0.01 versus WT Young control; ** or .dagger. P<0.01 versus WT and Dn-Trx Young+ACh, .dagger..dagger. P<0.01 versus WT and DnTrx-Tg Aged+ACh.

NO release in ex vivo mesenteric arteries is increased in Trx-Tg mice. NO release in en face superior mesenteric arteries from young and aged WT, Trx-Tg and dnTrx-Tg mice was measured using the NO probe DAF-FM followed by fluorescence microscopy. No statistically significant differences in basal NO release was measured in arteries derived from young mice. Incubation with ACh (10 μM) resulted in a rapid increase in green fluorescence, which was statistically significantly increased in Trx-Tg mice compared to WT and dnTrx-Tg mice. Pharmacological inhibition of NO synthesis using L-NAME (100 μM) prevented this increase in green fluorescence. Basal levels of NO release was unaltered with advanced aging in all mice groups and stimulation with ACh did not result in a significant increase in green fluorescence in arteries from aged WT and aged dnTrx-Tg mice. This in contrast to arteries from aged Trx-Tg mice, which clearly show a much higher fluorescent intensity after stimulation with ACh. Again, L-NAME blunted this effect.

Western blot of eNOS dimer and monomer in membrane fraction and cytosolic fraction of pooled mesenteric arteries from 6 young and 6 aged WT, Trx-Tg and dnTrx-Tg mice. Actin was used as internal standard. Densitometry analysis of eNOS dimer: monomer ratio from the blot. The ratio of dimer to monomer was normalized to the pixel intensity of actin. The ratio in young WT mice was set to 1. eNOS monomer was normalized to actin and set to 1 for WT Young mice. Densitometry analysis of eNOS monomer fragment of approximately 100 kDa. eNOS monomer fragment was normalized to actin and set to 1 for WT Young mice.

NO release is triggered via eNOS dimer activation. eNOS levels were determined in both the membrane and cytosolic fraction from pooled (from n=6 mice) mesenteric arteries. The eNOS dimer was detected only in the membrane fraction under reducing conditions. Whereas in the cytosolic fraction predominantly the monomeric eNOS form was detected. The dimer to monomer ratios were calculated from the membrane fraction normalized to the actin band and compared to WT young mice. eNOS dimer:monomer ratio that decreases with aging in WT and dnTrx-Tg mice, but is maintained in Trx-Tg mice. In the cytosolic fraction, eNOS monomerization was increased with aging in WT and dnTrx-Tg mice, but unaltered in Trx-Tg mice, suggesting that Trx-1 maintains the eNOS dimerization for the increased NO production to protect endothelial dysfunction. Interestingly, a band of approximately 100 kDa predominantly appeared in cytosolic fractions of mesenteric arteries from aged dnTrx-Tg mice and that can be fairly detected in young mice.

Since aging is associated with increased oxidative stress we reasoned that Trx-1 could act as an antioxidant protein that would have beneficial effects on preserving endothelial function with advancing age. A method of carboxymethylation was used to dissociate between reduced and oxidized Trx-1. Fully reduced Trx-1 is carboxymethylated on two of its sulfhydryl groups located on two cysteine residues and migrates as a dicarboxylated band, and fully oxidized Trx-1 remains noncarboxylated and migrates slower on a native gel. Total human Trx-1 levels (molecular weight is 12 kDa) in mesenteric artery lysates from pooled mice are shown. Faint bands corresponding to endogenous murine Trx-1 are shown for both young and aged WT bands. During aging the amount of total Trx-1 is slightly reduced compared to young mice. Mesenteric homogenates derived from young Trx-Tg mice, Trx-1 exists as both the reduced and oxidized form, with the reduced form being more predominant. In samples derived from aged Trx-Tg mice, the level of the reduced Trx-1 is lower compared to its young counterparts. In dnTrx-Tg mice, Trx-1 exists only in its oxidized form.

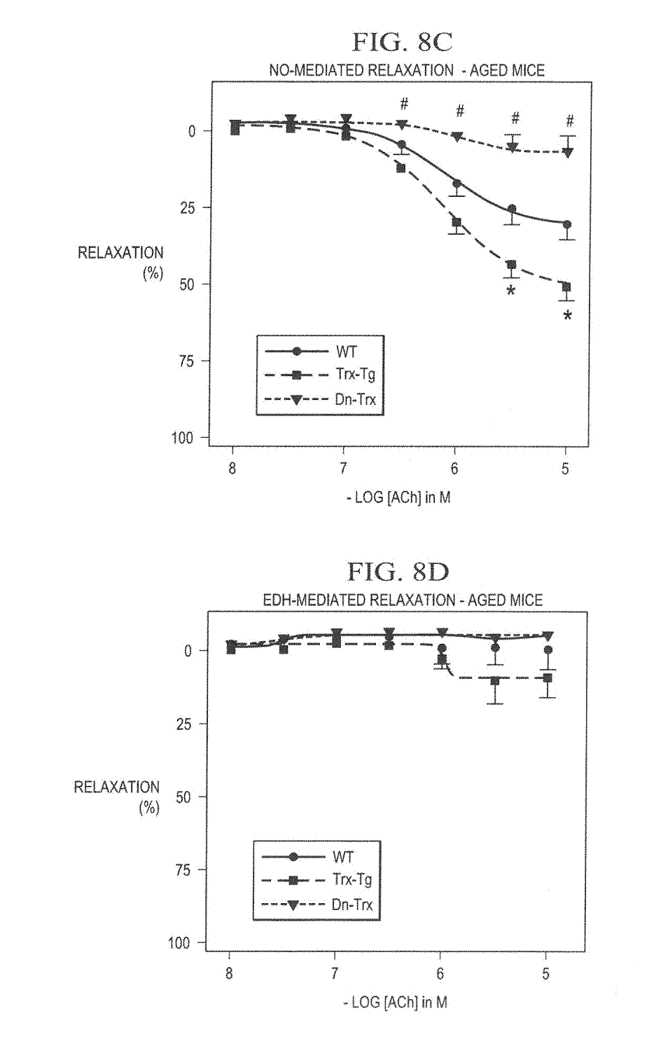

FIGS. 10A-10F show aging-induced endothelial dysfunction is restored by exogenous DTT and catalytically active h-Trx-1 in SMA from WT mice. Endothelium-dependent ACh-mediated relaxations in SMA derived from young WT (FIG. 10A), aged WT (FIG. 10B), Trx-Tg (FIG. 10C), and dnTrx-Tg (FIG. 10D) mice in the absence (CON) and presence of DTT (100 μM). (FIG. 10E) SMA from aged WT mice were incubated with INDO (10 μM), TRAM-34 (1 μM), and UCL1684 (1 μM) for 30 min in order to assess NO-mediated relaxing responses in the absence (CON) and presence of DTT (100 μM). (FIG. 10F) Cultured SMA were co-cultured with either a cocktail of catalytically active and recyclable h-Trx-1 (h-Trx-1+TR+NADPH), h-Trx-1+TR, h-Trx-1, or vehicle (None). SMA were contracted with a submaximal concentration of PHE before assessing relaxing responses to cumulative concentrations of ACh (0.01-10 μM). Values are means±SEM (n=3 mice). * P<0.05 versus CON, ** P<0.05 versus h-Trx-1+TR+NADPH. Since the active reduced Trx-1 is absent in dnTrx-Tg mice we questioned whether the protective effects in Trx-Tg mice were mediated by the sulfhydryl reducing property of reduced Trx-1. To address this we used the chemical sulfhydryl modifying agent DTT (100 μM) to mimic the effect of Trx-1. The aging-induced impairment in ACh-mediated relaxation in SMA from WT mice could be inhibited by incubating SMA for 30 min with DTT in the wire-myograph. Since the oxidized Trx-1 in dnTrx-Tg mice is mutated it cannot be reduced by DTT, but the endogenous murine Trx-1 can still be reduced by DTT. In SMA from young WT mice, DTT (100 μM) did not have an effect in modulating endothelium-dependent ACh-mediated relaxation (FIG. 10A). However, the same concentration of DTT restored ACh-mediated relaxations to comparable levels as in young mice (FIG. 10B). Sensitivity was statistically significantly increased in the presence of DTT (6.90±0.15) compared to in the absence of DTT (6.60±0.07). Strikingly, the sensitivity to ACh in the presence of DTT was not statistically significantly different from young mice (7.02±0.11; FIG. 10A). DTT had no effect in aged Trx-Tg mice (FIG. 10C), but tended to improve in dnTrx-Tg mice (FIG. 10D). DTT was able to improve NO-mediated responses in SMA from aged WT mice (FIG. 10E). Finally, we questioned whether culturing SMA overnight with h-Trx-1 would reverse the aging-induced endothelial dysfunction. Only the cocktail of h-Trx-1, TrxR and NADPH significantly improved NO-mediated relaxing responses (FIG. 10F). Interestingly, depletion of NADPH and h-Trx-1 alone resulted in comparable relaxing responses as SMA in the absence of h-Trx-1, indicating that the reduced h-Trx-1 is likely responsible for increased NO-mediated signaling.

### Discussion of Examples 2-3

The results above provided compelling evidence to support that in transgenic mice, overexpressing the human sulfhydryl reducing Trx-1 protein (Trx-Tg), aging-induced endothelial dysfunction is protected compared to WT and mice expressing a dominant-negative mutant human Trx-1 (dnTrx-Tg). This observation is based on the following key findings include maintained NO-mediated relaxing responses, preserved functional NO release, protection of eNOS dimerization, and prevention of eNOS monomerization in superior mesenteric arteries derived from aged Trx-Tg mice. In addition we show that the chemical reducing agent DTT mimics the effect of Trx-1, suggesting that Trx-1 exerts these beneficial effects via its disulfide-reducing properties.

Contractile responses were markedly reduced in superior mesenteric arteries from aged dnTrx-Tg mice compared to both aged WT and Trx-Tg mice, as evidenced by a lower tension generated by a depolarizing high potassium chloride solution and in response to the al-adrenergic receptor agonist phenylephrine. Sensitivity to phenylephrine was not decreased with aging in all three mice groups. Vascular adrenergic nerve function has been shown not to decline in the rat mesenteric arterial bed, which is in agreement with the present inventor's observations in mice. In small mesenteric arteries of the rat, the maximal tension in response to noradrenaline was comparable between young and aged animals, which are agreement with the findings in WT and dnTrx-Tg mice, but not Trx-Tg mice. However, this is the first study that reports aging-induced changes in murine superior mesenteric arterial structure and function. Internal diameters of superior mesenteric arteries were increased with aging, presumably due to enhanced blood flow demand in the gut with aging resulting in flow-induced arterial outward remodeling. Similarly, aging-induced increases in lumen diameter of murine second-order mesenteric arteries and rat carotid arteries have been reported. Despite this outward remodeling, maximal tensions to phenylephrine were comparable for young and aged WT and dnTrx-Tg mice, but increased in aged Trx-Tg mice compared to young Trx-Tg mice. One possible explanation for the unaltered contractility with aging in superior mesenteric arteries from WT and dnTrx-Tg mice might be the blunted aging-induced outward remodeling compared to Trx-Tg mice, which might imply a reduced wall mass and hence a decreased smooth muscle cell contractile capacity. NO plays a major role in arterial outward remodeling. In a surgical model of increased blood flow in the mesenteric artery increased eNOS expression and increased outward remodeling was observed, which was prevented by chronic eNOS inhibition by L-NAME and in eNOS knockout mice. It can therefore be anticipated that in aged WT and especially aged dnTrx-Tg mice, either functional NO release by shear stress is reduced and/or blood flow is decreased due to blunted NO-mediated vasodilatation. Both scenarios will result in blunted outward arterial remodeling. Cardiovascular diseases, such as hypertension, atherosclerosis, coronary artery disease, and also aging are characterized by endothelial dysfunction. In large arteries, where NO is the main vasorelaxing factor, the impairment in endothelium-dependent relaxation is manifested by a decrease in NO bioavailability, due to increased oxidative stress that uncouples the eNOS protein. Here, we confirm by multiple techniques that the aging-induced endothelial dysfunction is due to a reduction in NO bioavailability. The present inventor demonstrated a reduced ACh-mediated relaxing response in superior mesenteric arteries in aged mice compared to young mice, which was specific to the endothelium, since relaxing responses to the NO donor sodium nitroprusside, which activates soluble guanylate cyclase located in the smooth muscle cells, was unaltered with aging. When vasoactive prostanoids synthesis and the endothelium-dependent hyperpolarizing response were pharmacologically inhibited, the residual NO-mediated relaxations were decreased with aging in both WT and dnTrx-Tg mice, but were preserved in Trx-Tg mice. In addition, blunted endothelium-dependent hyperpolarizing (EDH) relaxing responses were observed with aging, which is in agreement with earlier observations in the rat mesenteric arterial bed. In superior mesenteric arteries from Trx-Tg mice, these EDH responses were less blunted compared to their WT and dnTrx-Tg counterparts.

The fluorescent NO probe DAF-FM was used to detect functional NO release in response to exogenously added ACh in en face arterial preparations. The specificity of this fluorescent technique was shown by the observation that the NOS inhibitor L-NAME prevented the NO release. Interestingly, a higher NO release was detected in arteries from young Trx-Tg mice compared to WT and dnTrx-Tg mice. Blunted NO release with aging was observed in arterial preparations from WT and dnTrx-Tg mice, which was preserved in Trx-Tg mice. These observations confirm the present inventor's findings that vascular reactivity results in that the ability of the endothelial cells in superior mesenteric arteries from Trx-Tg mice to generate NO in response to muscarinic receptor activation is protected with advanced age.

The above observations clearly show a reduced NO bioavailability with aging in WT and dnTrx-Tg mice, which was sustained in Trx-Tg mice. First we addressed the possibility that a reduced NO bioavailability might result from an increased eNOS monomerization. The active form of eNOS enzyme exists as two identical subunits and hence is located as a dimer in caseload. It has been shown that cysteines 94 and 99 of eNOS form a zinc tetra-coordinated (ZnS₄) cluster between each subunit. This ZnS₄ cluster is highly sensitive to oxidants such as ONOO+, NO, and H₂O₂ and the oxidation of this ZnS₄ cluster results in monomerization of eNOS and inhibition of catalytic activity. eNOS dimer and monomer forms have been detected using low-temperature SDS-PAGE using pooled protein lysate samples from the membrane fraction from 6 mice of each experimental group. The ratio between eNOS dimer to monomer was decreased in mesenteric arteries from aged WT and dnTrx-Tg mice, but was maintained in Trx-Tg mice. This reduction in eNOS dimers to monomers is in agreement with an earlier report studying endothelial dysfunction in mesenteric arteries from aged C57BL/6J mice. In the cytosolic fraction, increased eNOS monomerization was observed with aging in WT and dnTrx-Tg mice, but not in aged Trx-Tg mice. Concomitant with this increased eNOS monomerization an increased band of approximately 100 kD appeared in the mesenteric artery cytosolic fraction from aged dnTrx-Tg mice. This could be a cleaved eNOS product due to intracellular processing. Overall these data show that functional Trx-1 preserves eNOS dimerization, presumably via preventing the oxidation of the ZnS₄ cluster.

The present invention provides the administering of thioredoxin-1 to a subject in need of treatment for hypertension. Thioredoxin-1 is a different composition that thioredoxin-2 with different properties and characteristics. This is further illustrated in the fact that thioredoxin-1 functions differently and through a different pathway than thioredoxin-2. Thioredoxin-2 is mitochondrial and is not permeable into cells. It is transported to mitochondria by a transport mechanism. As a protein it cannot enter cells to have therapeutic effect. In contrast, thioredoxin 1 is cell permeable. From the data herein, the dnTrx mice have low levels of thioredoxin-1, but have regular levels of thioredoxin-2. But these mice show hypertension without any treatments such as angiotensin II infusion. Thus, removing thioredoxin-1 causes hypertension (dnTrxTg) and supplementing thioredoxin-1 decreases hypertension (Trx-Tg). Additionally, they specifically expressed Trx2 in the endothelial cells. Global expression of Trx supports is the present invention's method of injecting Trx by a route and dose that will go to every organ.

### Example 4

### Aged NT and dnTrx-Tg Mice are Hypertensive, but Aged Trx-Tg Mice are Normotensive

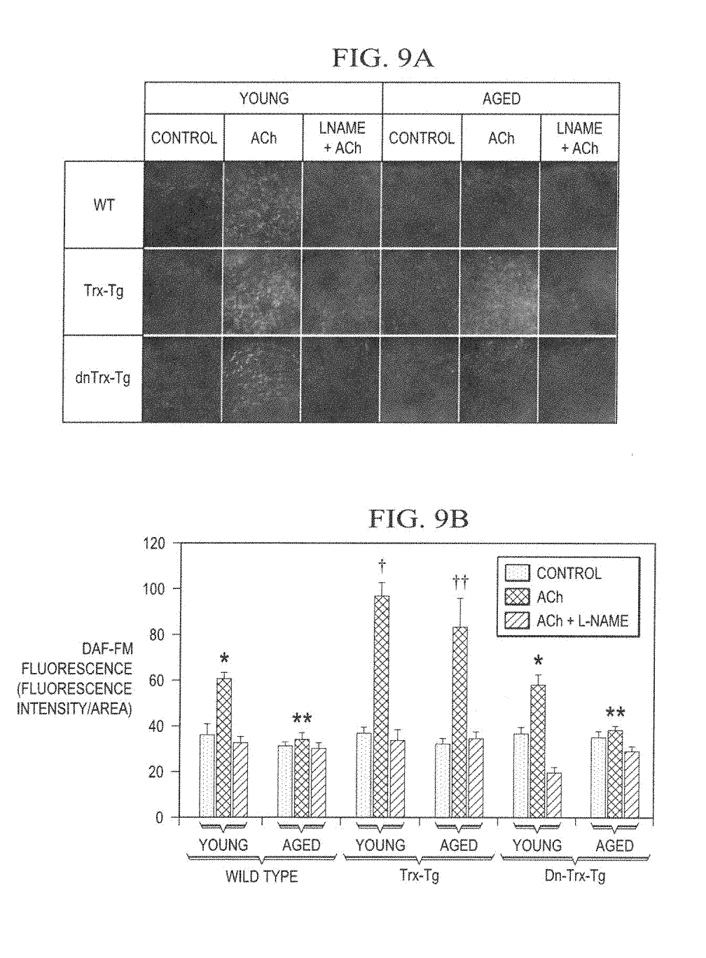

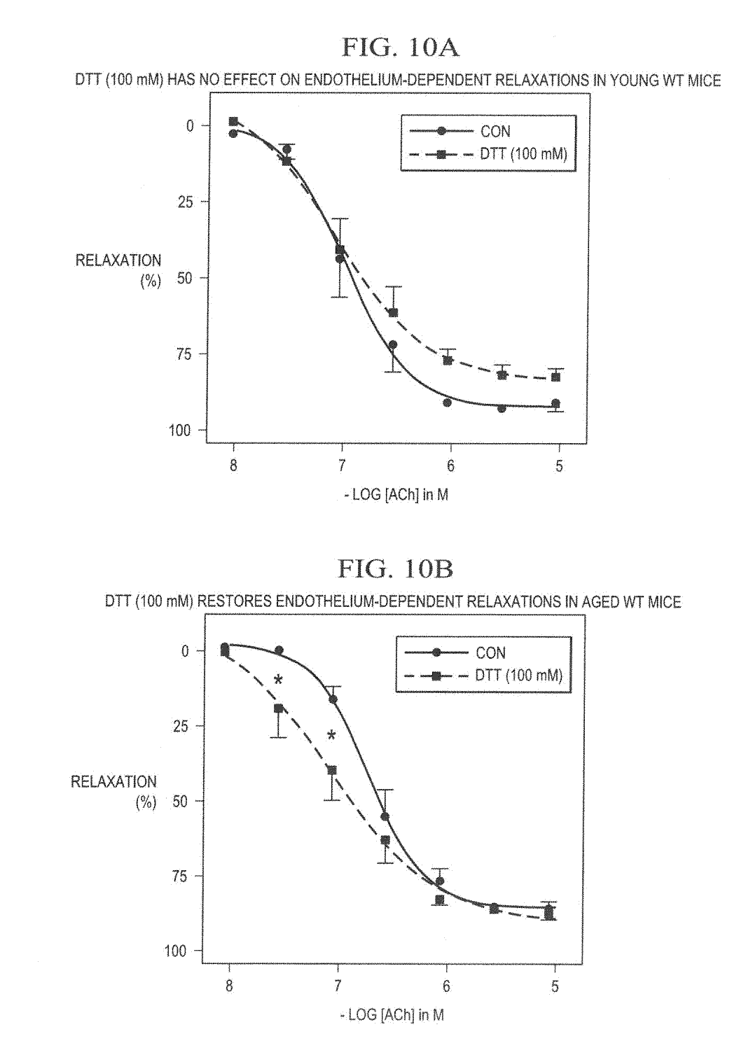

To evaluate the effect of Trx on age-related hypertension, we generated mice overexpressing human Trx (Trx-Tg) and another strain expressing mutant (C32S, C35S) human Trx (dnTrx-Tg) (Das, 2015, supra; Hilgers, 2015 supra). The overexpression of mutant Trx decreases the level of active Trx in a dominant-negative manner by competing with thioredoxin reductase (TrxR) for its reduction (J. E. Oblong et al., supra). The activity of Trx was significantly decreased in carotid arteries (CA) from young dnTrx-Tg mice compared to NT or Trx-Tg mice (FIG. 111A). As expected, CA of young Trx-Tg mice showed higher Trx activity than NT mice. Aging was associated with a marked loss of Trx activity in CA of NT mice and persistently low Trx activity in CA of aged dnTrx-Tg mice. In contrast, the activity of Trx in CA from aged Trx-Tg mice was enhanced compared to all CA preparations (FIG. 11A). The activity of TrxR was not significantly different between young or aged NT, Trx-Tg or dnTrx-Tg mice (FIG. 11B). Additionally, in CA of NT mice, Trx remained in a reduced state in young mice, but increased level of Trx was oxidized during aging (FIG. 11C, upper panel and FIG. 12 A-12B).

The redox state of Trx in CA and aortae of young and aged Trx-Tg mice revealed similar high levels of reduced Trx (FIG. 11D and FIG. 12 A-12B). In contrast, CA and aortae of young and aged dnTrx-Tg mice showed high levels of oxidized Trx and sparse reduced Trx (FIGS. 11C&11D and FIGS. 12A&12B). These data show that aging is associated with a marked shift from a reduced to an oxidized vascular redox state, and reveal that increased Trx levels during aging maintain a vascular redox state similar to younger mice. From a redox view point, vessels of aged Trx-Tg mice appeared similar to young Trx-Tg or young NT mice, but not young dnTrx-Tg mice (FIG. 12A-12B). The level of mitochondrial Trx2 did not change in the superior mesenteric artery (SMA) of aged NT, Trx-Tg or dnTrx-Tg mice (FIG. 11E, middle panel), suggesting that Trx2 expression is not modulated due to high levels of Trx in young or aged mice/

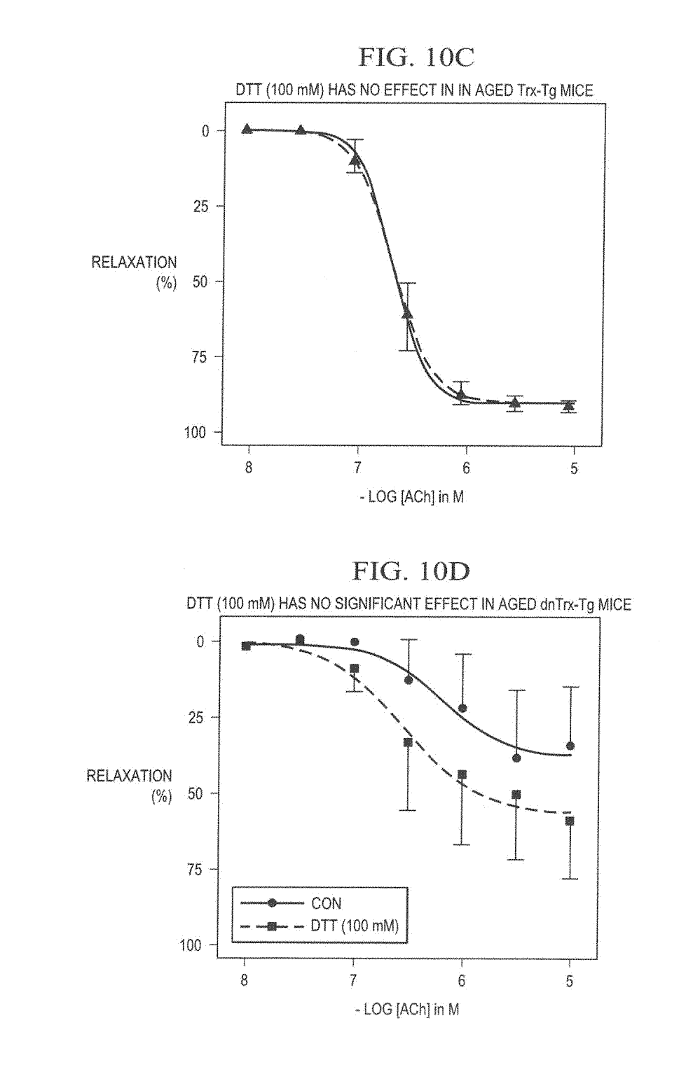

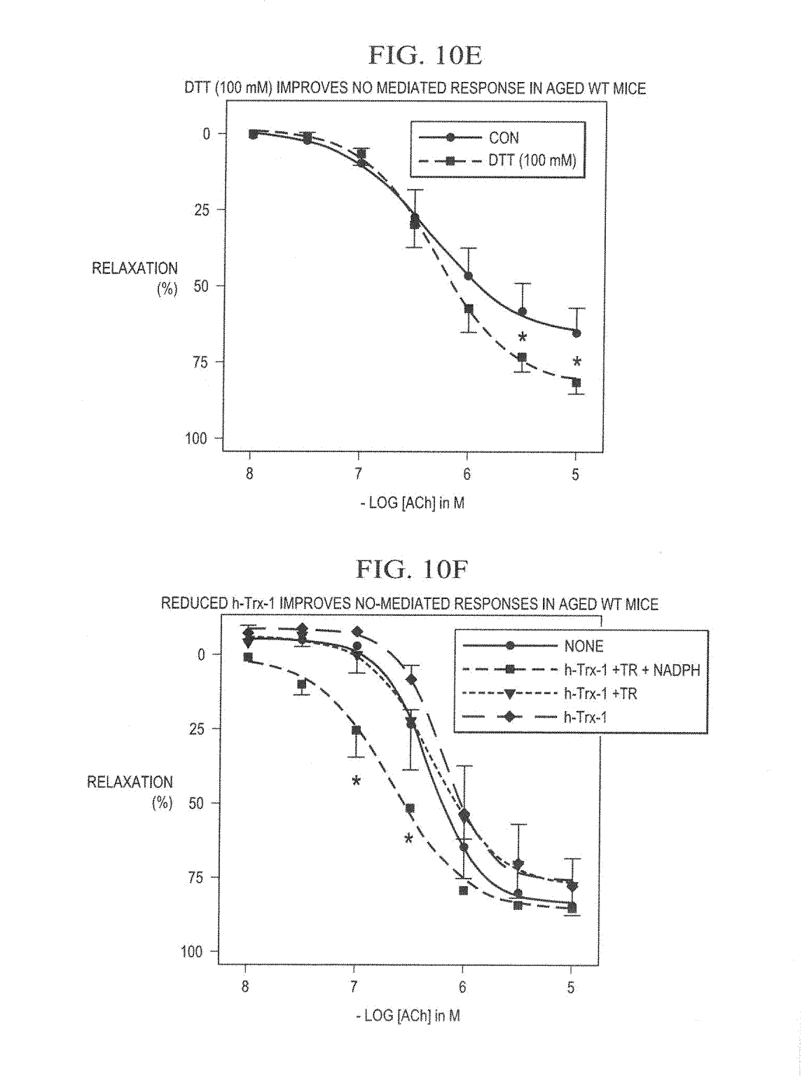

A study was conducted to evaluate whether high levels of Trx alter mean arterial pressures (MAP) between young and aged mice using radio-telemetry (FIG. 13A-C). MAP of young and aged Trx-Tg mice were lower than age-matched NT mice (FIG. 13F). Additionally, whereas aged NT mice exhibited age-dependent hypertension, aged Trx-Tg mice remained normotensive and there was no significant difference between young NT mice and aged Trx-Tg mice (FIG. 11F). The MAP of aged dnTrx-Tg was significantly higher than the aged Trx-Tg. Additionally, although the aged dnTrx-Tg mice showed about 5 mmHg higher MAP compared to aged NT mice, this increase was not statistically significant. Both systolic (FIG. 11G) and diastolic (FIG. 11H) blood pressure remained lower in aged Trx-Tg mice compared to either NT or dnTrx-Tg. Taken together, the present data demonstrate that aged Trx-Tg mice were normotensive, whereas aged NT or dnTrx-Tg mice developed age-related hypertension. Surprisingly, the heart rate of aged NT or dnTrx-Tg mice was significantly lower compared to aged Trx-Tg mice (FIG. 14A). However, there was no difference in the heart rate of young Trx-Tg and aged Trx-Tg mice (FIG. 14A. As shown in FIG. 14B the stiffness index for aged Trx-Tg mice was significantly lower compared to aged NT or dnTrx-Tg mice. Further, there was no statistical difference in the stiffness index of young NT or aged Trx-Tg mice, indicating that high levels of Trx prevent arterial stiffness in aging.

### Example 5

### Treatment of Aged Mice with rhTRX Reverses Age-Related Hypertension

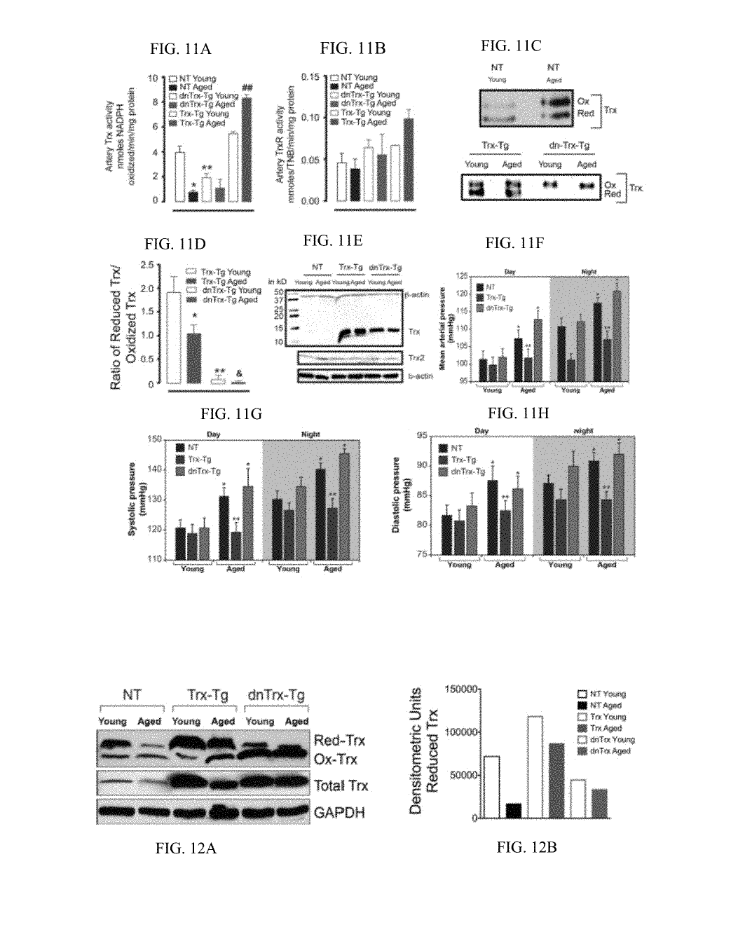

Since Trx-Tg mice have high levels of hTrx at birth, lower MAP in these animals suggests protection against development of hypertension during aging, but does not show whether it is effective in aged mice that already have hypertension. To determine whether rhTrx would be effective in reversing age-related hypertension, rhTrx was injected via tail vein to aged male WT mice (2.5 mg/kg) at 48 hours intervals for 3-consecutive days. After the third injection blood pressure was recorded after 1 and 12 days using radio-telemetry. Telemetry recordings for MAP, heart rate and activity of saline and rhTrx injected aged WT mice are presented in FIG. 15A. As shown in FIGS. 15B and 15C, the MAP of aged WT mice was significantly decreased to the level seen in young WT mice. During daytime a drop of 15 mmHg was measured and during nighttime a drop of 18 mmHg was measured at the end of 12 days (FIGS. 15B&C). Both systolic and diastolic blood pressure was decreased in WT mice injected with rhTrx (FIGS. 15D&E). Intriguingly, blood pressure remained lower for at least 20-consecutive days after the last rhTrx injection (FIGS. 15B and D), demonstrating prolonged efficacy of rhTrx in maintaining a lower blood pressure in these mice. Further, the stiffness index was significantly lower in rhTrx treated mice compared to saline injected mice, demonstrating restoration structural elements of vessel due to aging (FIG. 15F. High level of injected rhTrx was detected in the heart (FIG. 15G), and in the blood plasma (FIG. 15H) 24 h after the third injection. The injected rhTrx was mostly found to be in reduced state in the plasma (FIG. 15H).

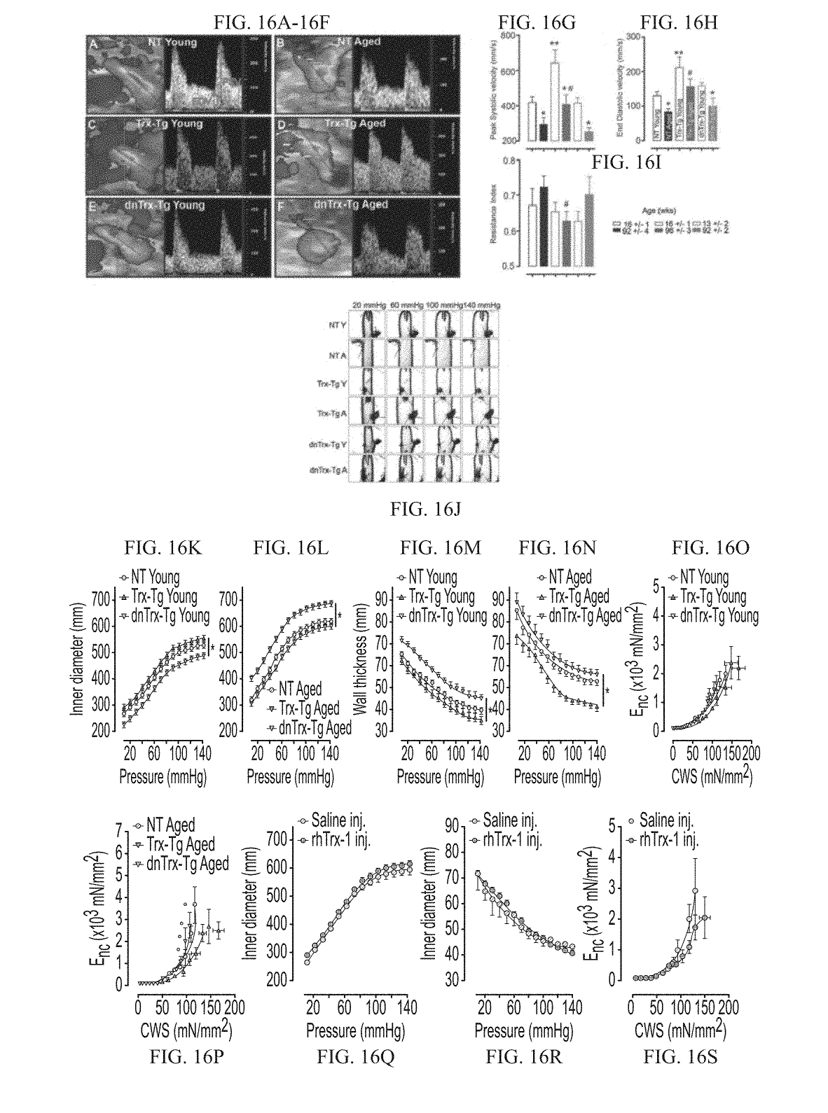

To determine whether rhTrx lowers blood pressure in aged mice in a similar fashion as an established mechanism such as, angiotensin receptor 1 (AT1)-dependent pathway, we injected losartan, an AT1 receptor blocker to aged WT mice and measured blood pressure over a period of 12 days using radio-telemetry. As shown in FIG. 18A, losartan did decrease the blood pressure in aged WT mice after the first dose as expected, but the blood pressure continued to rise with subsequent dosages of losartan or when losartan was withdrawn, and the blood pressure reached to the original level within 8-12 days (FIG. 18A). In the SMA losartan did not result in any structural changes in inner diameter (FIG. 18B), wall thickness (FIG. 18C), and elasticity FIG. 18D. This data indicates that lowering of blood pressure due to losartan is effective for a short duration, whereas Trx treatment resulted in prolonged lowering of blood pressure in aged mice.

### Example 6

### High Amounts of TRX Decrease SMA Vascular Flow Resistance

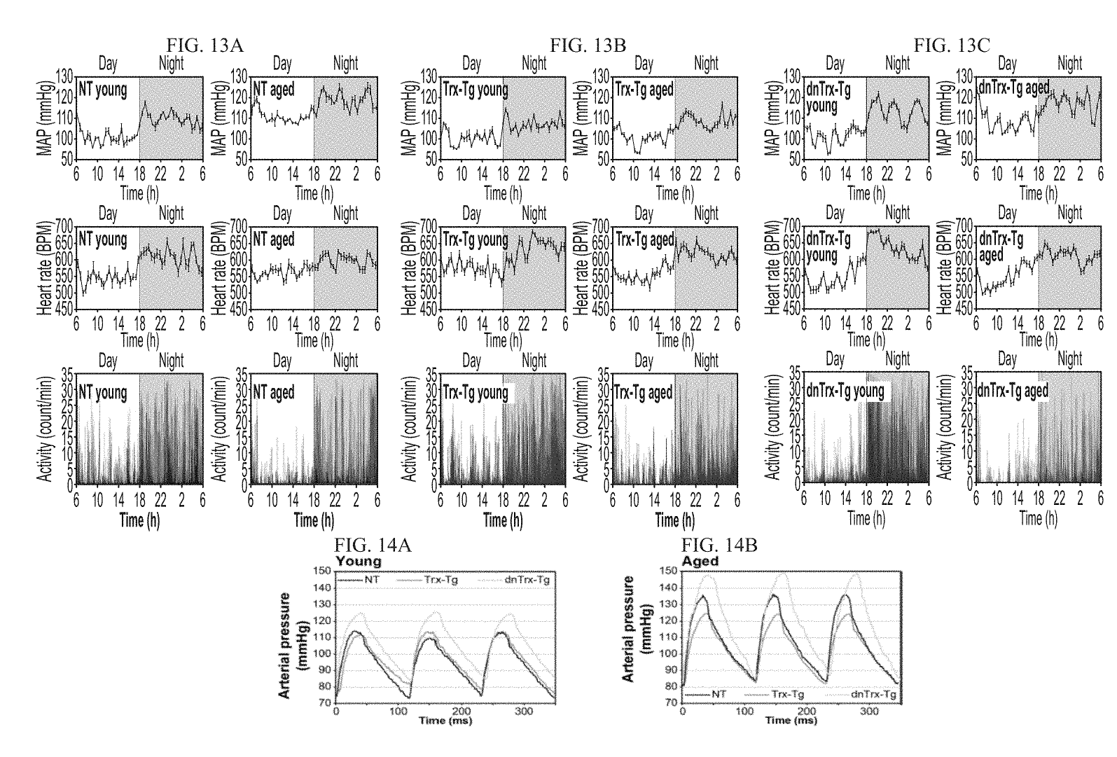

Evaluation was performed of age-related arterial abnormalities in the SMA, which is considered a large elastic artery (R M et al., Blood Vessels 23, 199-224 (1986)), would translate into altered mesenteric vascular resistance using high-resolution ultrasound imaging, which permitted calculations of peak systolic (PSV) and end diastolic (EDV) blood velocities, and vascular resistance index. SMA blood velocity profiles (FIG. 16A-F) revealed that PSV (FIG. 16G) and EDV (FIG. 16H) were higher in young and aged Trx-Tg compared to age-matched NT and dnTrx-Tg mice. The resistance index calculated as the difference between PSV and EDV divided by PSV, was comparable between young NT, Trx-Tg, and dnTrx-Tg mice, but increased with aging in NT, and not in aged Trx-Tg (0.629±0.026) mice (FIG. 16I).

Pressure-diameter relationships, pressure-wall thickness relationships and circumferential wall stress (CWS)-incremental elastic modulus (E_{inc}) relationships were determined in cannulated SMA of young and aged mouse strains. It was the inventor's hypothesis that overexpression of Trx alters age-related physiological outward remodeling and/or elasticity of the large elastic SMA, which could account for the preserved flow characteristics in the SMA of aged Trx-Tg mice. Representative images of cannulated SMA from all six mice groups pressurized at 20, 60, 100 and 140 mmHg are depicted in FIG. 16J. SMA inner diameters were comparable for young NT, Trx-Tg and dnTrx-Tg mice (FIG. 16K). Wall thickness was comparable for SMA derived from young NT and Trx-Tg mice, but was statistically significantly increased in SMA from young dnTrx-Tg (FIG. 16K). Elasticity, shown as CWS plotted against E_{inc}, was similar for SMA from young mice (FIG. 16O). The inner diameter of the SMA was increased in aged mice indicative of outward structural remodeling (FIG. 16L). This outward remodeling was greater in Trx-Tg mice compared to NT and dnTrx-Tg mice (FIG. 16A, 16L). In addition, we observed a thicker wall characteristic of hypertrophic remodeling with aging in SMA from NT and dnTrx-Tg mice, whereas SMA from Trx-Tg mice showed no change in wall thickness (FIG. 16N). FIG. 16P shows that the slope of the CWS versus E_{inc} curves were steeper in SMA from aged NT and dnTrx-Tg mice compared to Trx-Tg mice, indicating a greater stiffness of the SMA in aged NT and dnTrx-Tg compared to aged Trx-Tg mice. Pressure-diameter relationships of the SMA from aged NT mice that were injected with rhTrx were also checked. After 12 days following the 3^{rd} rhTrx dose we observed outward remodeling (FIG. 16Q) and increased elasticity (FIG. 16R) compared to saline-injected mice, but no change in wall thickness (FIG. 16S). Collectively, these studies demonstrate a profound effect of high levels of Trx in the maintenance of structural and functional characteristics of blood vessels during the development of aging process.

### Example 7

### Endothelium-Dependent Relaxation is Preserved in SMA of Aged Trx-Tg Mice but Severely Blunted in Aged dnTrx-Tg Mice

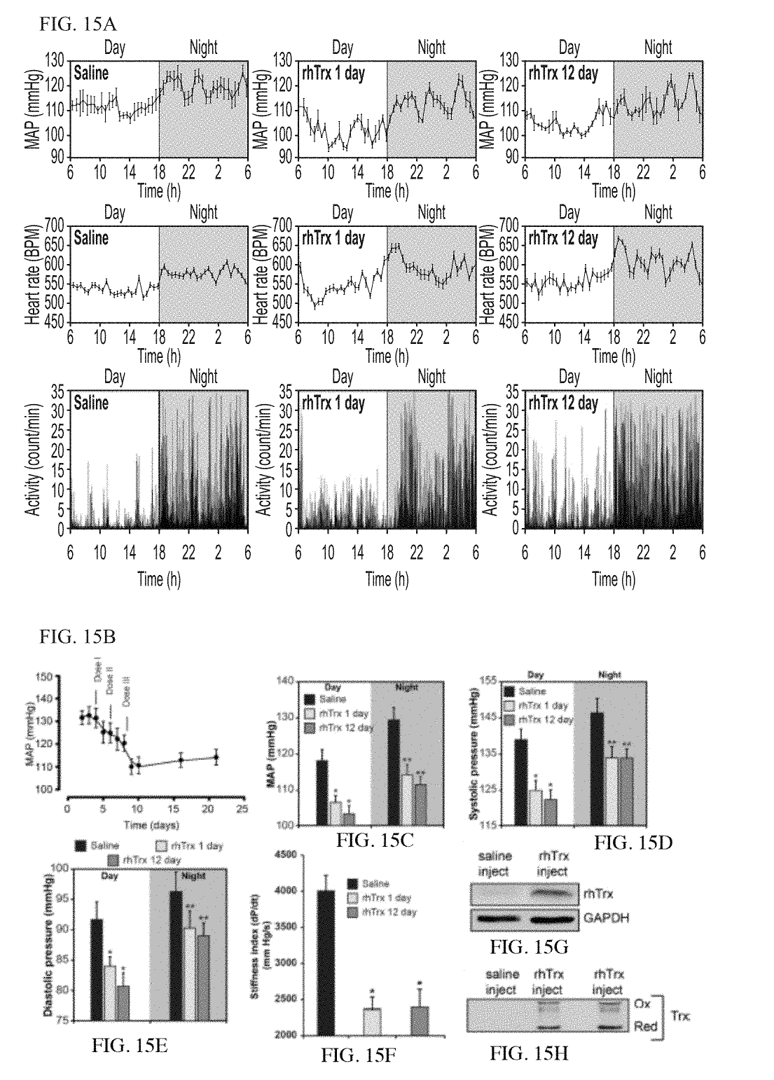

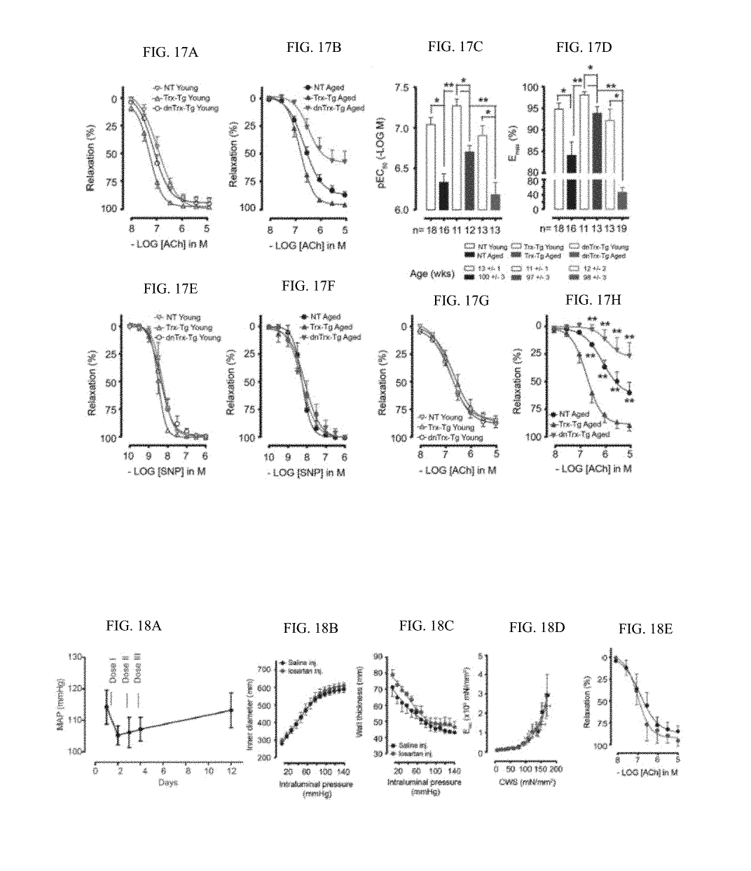

The vascular endothelium plays a crucial role in maintaining vasomotor tone and controlling blood pressure. Therefore, we determined whether Trx would impact endothelium-dependent relaxation. Trx was depleted in the SMA using an ex vivo siRNA transfection specific to Trx, and determined endothelium-dependent relaxing responses. As shown in FIG. 19A-B, loss of Trx impaired relaxation in the SMA demonstrating that Trx is required for an optimal vascular relaxing response. Next, we determined the effect of high levels of vascular Trx or functionally inactive Trx on endothelium-dependent relaxations. Endothelium-dependent acetylcholine (ACh)-mediated relaxations were comparable in SMA from young mice (FIG. 17A). Sensitivity to ACh (pEC₅₀) and maximal relaxations to 10 μM ACh (Eₘₐₓ) was higher in SMA from young Trx-Tg mice compared to young NT and young dnTrx-Tg mice, but this did not reach statistical significance (FIGS. 17C&4D). Aging was associated with impaired ACh-induced relaxation in SMA from all mice groups (FIG. 17B). However, this effect was markedly less in Trx-Tg mice compared to NT and especially in dnTrx-Tg mice, as shown by the greater drop in pEC₅₀ and Eₘₐₓ values in the latter two mice groups (FIGS. 17C&17D). This age-related endothelial dysfunction observed in SMA from aged mice appeared to be specific to the endothelium, since relaxing responses to the endothelium-independent NO donor sodium nitroprusside were comparable between SMA from young (FIG. 17E) and aged (FIG. 17F) mice.

Since NO is the major endothelium-derived vasorelaxing factor in the SMA, we determined the contribution of NO to ACh-mediated relaxations. To isolate NO-dependent relaxations, SMA were incubated with the cyclooxygenase inhibitor indomethacin (10 μM), and with TRAM-34 (1 μM) and UCL 1684 (1 μM) to inhibit the endothelial Ca^{2±}-activated K⁺ channels, K_{Ca}3.1 and K_{Ca}2.3, respectively, that mediate endothelium-dependent hyperpolarization (EDH) in this vascular bed. Comparable NO-mediated relaxations were observed in SMA from young mice (FIG. 17G). In SMA of Trx-Tg mice, NO-mediated relaxations were unaffected by advanced aging (FIG. 17H). However, SMA of aged NT and aged dnTrx-Tg mice showed a significant loss of NO-mediated relaxations compared to Trx-Tg mice (FIG. 17H).

### Example 8

### rhTRX Treatment Improves Endothelium-Dependent Relaxation in Aged Mice

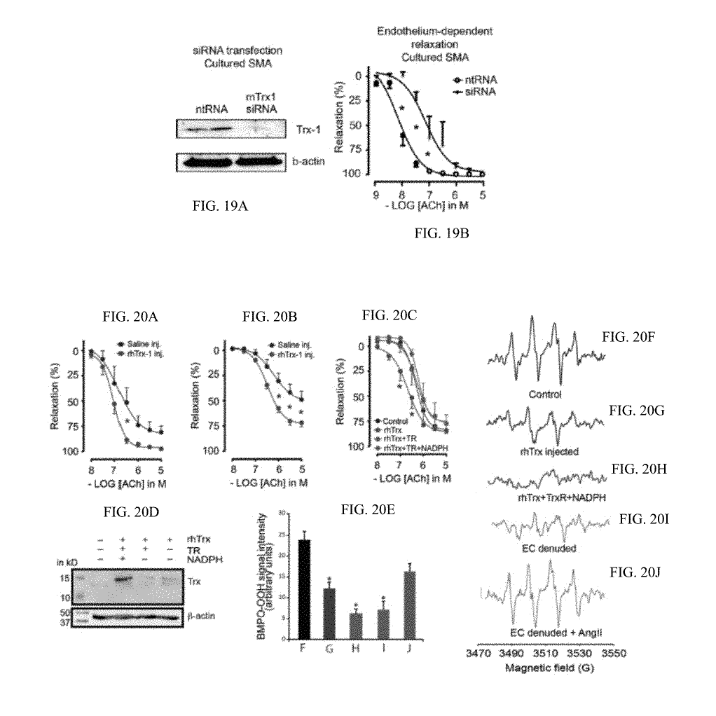

Since rhTrx treatment decreased hypertension in aged WT mice, we determined whether endothelial dysfunction is also ameliorated in these mice. As shown in FIG. 20A, rhTrx treatment significantly improved overall endothelium-dependent ACh-mediated relaxing responses. In SMA derived from rhTrx-injected mice, NO-mediated relaxing responses were statistically significantly greater compared to its saline-injected littermates (FIG. 20B). Collectively, these data suggest that injected rhTrx is therapeutically effective to lower MAP of aged mice via promoting endothelium-dependent relaxation.

The present inventor and colleagues have shown that rhTrx is internalized by epithelial or endothelial cells). Therefore, it was determined whether incubation of SMA from aged WT mice with the reduced form of hTrx could improve ACh-induced relaxations. NO-mediated relaxation was significantly improved after incubation of SMA in the reducing reaction (rhTrx+TrxR+NADPH) (FIG. 20C). However, incubation of SMA with only rhTrx or Trx+TrxR did not improve relaxations of aged SMA, indicating that only the reduced form of Trx (Trx-(SH)₂) is able to reverse impaired endothelium-dependent relaxations in aged SMA. Notably, when the level of rhTrx was evaluated in SMA incubated with Trx alone, or in combination with TrxR and NADPH, higher levels of the rhTrx were observed in SMA exposed to the entire reaction mixture (FIG. 20D). These data demonstrate that the Trx-(SH)₂ specifically restores relaxation in arteries of aged mice.

Since in vitro rhTrx did not improve vascular relaxation, but rhTrx in combination with TrxR and NADPH did result in relaxation of SMA, we speculated that applied rhTrx might have been oxidized due to ROS generated by the aged SMA ex vivo. Inclusion of a complete Trx redox cycling system would continuously regenerate Trx-(SH)₂ by converting oxidized Trx (Trx-5₂) to Trx-(SH)₂ in the presence of TrxR and NADPH. Based on this reasoning, we determined the vascular source of O₂.⁻ in thoracic artery segments (TAS) ex vivo using electron paramagnetic resonance spectrometry (EPR). As shown in FIGS. 20E&F, TAS from aged WT mice generated significant amounts of O₂.⁻ as determined by EPR spin-trapping techniques. However, segments from rhTrx-injected aged WT mice showed reduced levels of BMPO—OOH adduct (FIGS. 5E&F). Further, aged TAS from WT mice incubated with a Trx-reducing system showed marked decrease in O₂.⁻ production (FIGS. 5E&H). To determine the cellular source of O₂.⁻ in aged arteries we removed the endothelium from segments and determined the O₂ production using EPR. As shown in FIGS. 5G&E, denudation of EC from TAS decreased O₂.⁻ generation indicating a critical role of endothelium in age-related O₂.⁻ generation in TAS. As a positive control we added angiotensin II (AngII) to TAS segments without the endothelium and measured O₂.⁻ levels in response to AngII (FIGS. 5E&J). AngII treatment increased O₂.⁻ generation compared to endothelium denuded TAS. These studies suggest that the endothelium is the primary source of O₂.⁻ generation that can be attenuated by Trx in its reduced state in TAS from aged NT mice. It is important to mention here that AngII specifically induced O₂.⁻ generation in vascular cells other than endothelial, such as vascular smooth muscle cells.

### Example 9

### eNOS, but not NADPH Oxidase (NOX) is a Major Source of O₂.⁻ in Aged TAS of NT Mice

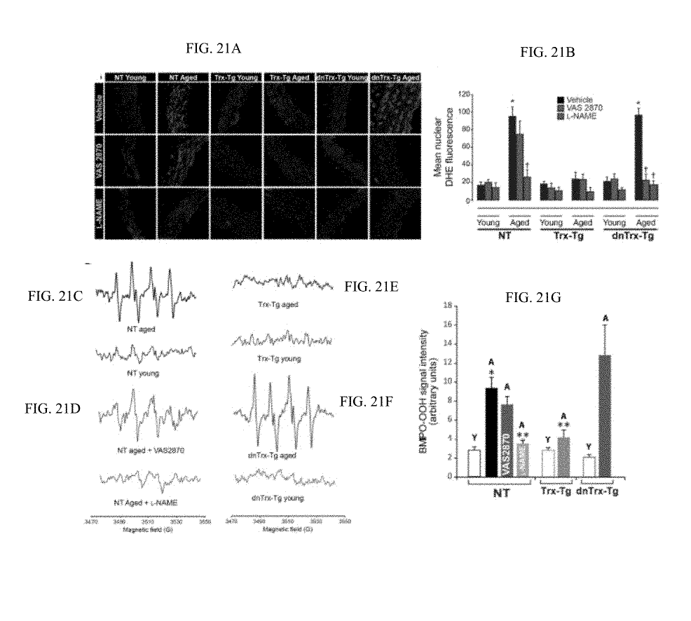

Given that uncoupling of eNOS is a major mechanism of production of vascular O₂.⁻, we determined whether dysfunctional eNOS in aged NT or dnTrx-Tg mice is a source of O₂.⁻. If eNOS were the source of O₂.⁻, then we would expect a decrease in O₂.⁻ production after treatment of TAS with the non-selective NOS blocker L-NAME. Alternatively, if NADPH oxidase (Nox) is the source of O₂.⁻ in aged NT or dnTrx-Tg mice, O₂.⁻ release should be reduced after VAS2870 treatment, a non-selective Nox inhibitor (Q A et al. Free Radic Biol Med 52:1897-1902 (2012). There was no difference in O₂.⁻ generation between TAS of young NT, Trx-Tg or dnTrx-Tg mice (FIG. 21A). However, TAS of aged NT mice showed higher levels of nuclear DHE staining suggestive of increased O₂.⁻ generation; this signal was decreased by L-NAME (FIGS. 21A&B). In contrast, VAS2870 did not decrease DHE staining in TAS of aged NT (FIGS. 21A&B). These data suggest that eNOS underlies O₂.⁻ generation in aortae of aged NT mice. Interestingly, the minimal DHE staining in TAS of young or aged Trx-Tg mice was marginally decreased by L-NAME, suggesting that TAS of aged Trx-Tg mice only nominally generate O₂.⁻. Surprisingly, DHE staining in TAS of aged dnTrx-Tg mice was decreased with either VAS2870 or L-NAME, indicating that O₂.⁻ is released by both Nox and eNOS in these mice (FIGS. 21A&B). To conclusively establish the O₂.⁻ release by eNOS in aged TAS, we utilized EPR spin-trapping to specifically detect O₂.⁻. Strong BMPO—OOH adducts (signifying O₂.⁻) were detected in TAS of aged NT (FIGS. 21C&G) and dnTrx-Tg mice (FIGS. 21F&G), but this adduct was markedly absent in TAS of Trx-Tg mice (FIGS. 6E&G). The magnitude of BMPO—OOH adduct did not decrease in the presence of non-specific Nox inhibitor VAS2870, but TAS treated with L-NAME demonstrated significant decrease in BMPO—OOH adduct suggesting eNOS, but not Nox is involved in increased O₂.⁻ generation in aged NT mice.

### Example 10

### NO Release is Preserved in Ex Vivo Coronary Arteries from Aged Trx-Tg Mice

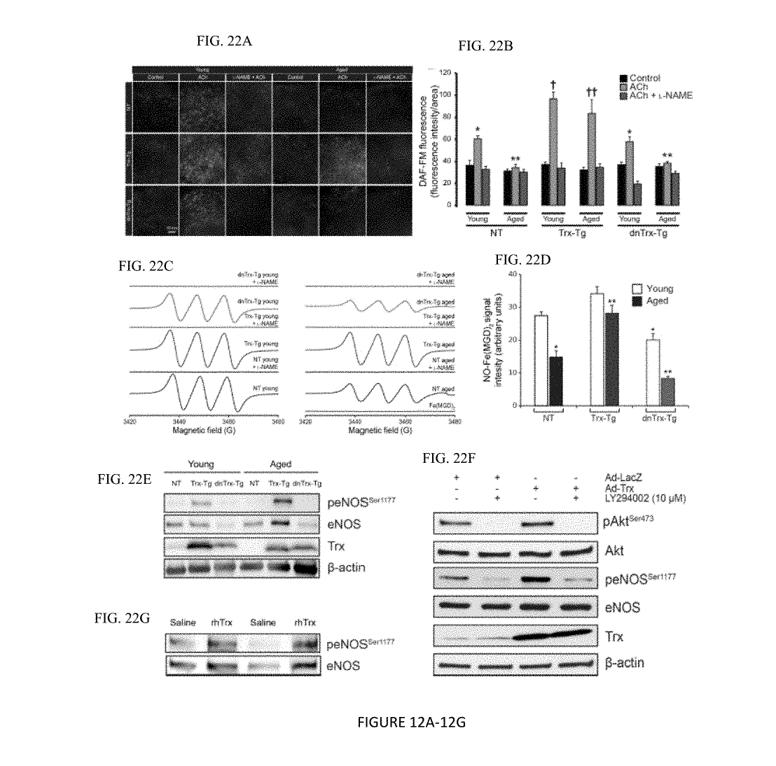

NO release was measured in en face CA from young and aged NT, Trx-Tg and dnTrx-Tg mice using the NO probe DAF-FM followed by fluorescence microscopy. Basal NO release was similar between CA from young mice (FIGS. 22A&B). Incubation with ACh (10 μM) rapidly increased green fluorescence; the signal intensity was higher in CA from young Trx-Tg mice compared to young NT or young dnTrx-Tg mice (FIGS. 22A&B). Inhibition of NO synthesis by L-NAME prevented the ACh-induced signal (FIGS. 22A&B). Basal NO release also was similar between CA from aged mice (FIGS. A&B). In contrast, ACh did not significantly increase green fluorescence in CA from aged NT or dnTrx-Tg mice, but markedly increased the signal in aged Trx-Tg mice (FIGS. 22A&B). Since DAF-FM measures the NO oxidation product N₂O₃, but not NO, we directly measured NO production using EPR spectroscopy. As shown in FIGS. 22C&D, NO production was significantly decreased in aged NT mice, but increased in aged Trx-Tg mice compared to aged NT mice. The NO release from CA of aged dnTrx-Tg mice was significantly lower compared to either aged NT or aged Trx-Tg mice. These data directly provide evidence that NO release is acutely decreased in aged NT and dnTrx-Tg mice, but not in aged Trx-Tg mice. Collectively, the present data confirm that high levels of Trx protect against loss of age-related NO release, and conversely, oxidation of Trx accentuates this age-related NO compromise.

### Example 11

### eNOS Phosphorylation and Expression are Increased in Mesenteric Arteries of Aged Trx-Tg Mice but not in SMA of Aged NT or dnTrx-Tg Mice

Next, the possible mechanism by which eNOS becomes dysfunctional in aged mice was determined. Since NO release in response to ACh persisted in SMA of aged Trx-Tg mice, but not in aged NT or dnTrx-Tg mice, we evaluated whether activation of eNOS in mesenteric arteries (MA) differed between the mice groups. Constitutively phosphorylated eNOS^{Ser1177} was prominent in MA of young and aged Trx-Tg mice, but not in age-matched NT or dnTrx-Tg mice (FIG. 22E, top panel). Thus, the basal activity of eNOS was enhanced in MA of young and aged Trx-Tg mice. The expression of eNOS protein was positively correlated with Trx abundance in MA of Trx-Tg mice (FIG. 22E). In contrast, eNOS expression and phosphorylation was decreased in MA of dnTrx-Tg mice (FIG. 22E). An in vitro assay was employed to explore whether Trx could activate an upstream kinase resulting in eNOS phosphorylation. In these studies, overexpression of Trx in human coronary artery endothelial cells (HCAEC) increased phosphorylation of Akt, suggesting its activation by Trx (FIG. 22F), which was correlated with increased phosphorylation of eNOS^{Ser1177}. Additionally, inhibition of Akt activation by LY294002 (10 μM) inhibited eNOS^{Ser1177} phosphorylation by Trx (FIG. 22F). When we injected rhTrx to aged WT mice, eNOS expression and phosphorylation of eNOS^{Ser1177} were increased in MA (FIG. 22G), indicating that chronic administration of rhTrx protects against age-related eNOS dysfunction. Overall these studies demonstrate that Trx induces eNOS^{Ser1177} phosphorylation via Akt activation.

### Example 12

### eNOS is S-Glutathionylated in Aged NT Mice but not in Aged Trx-Tg Mice

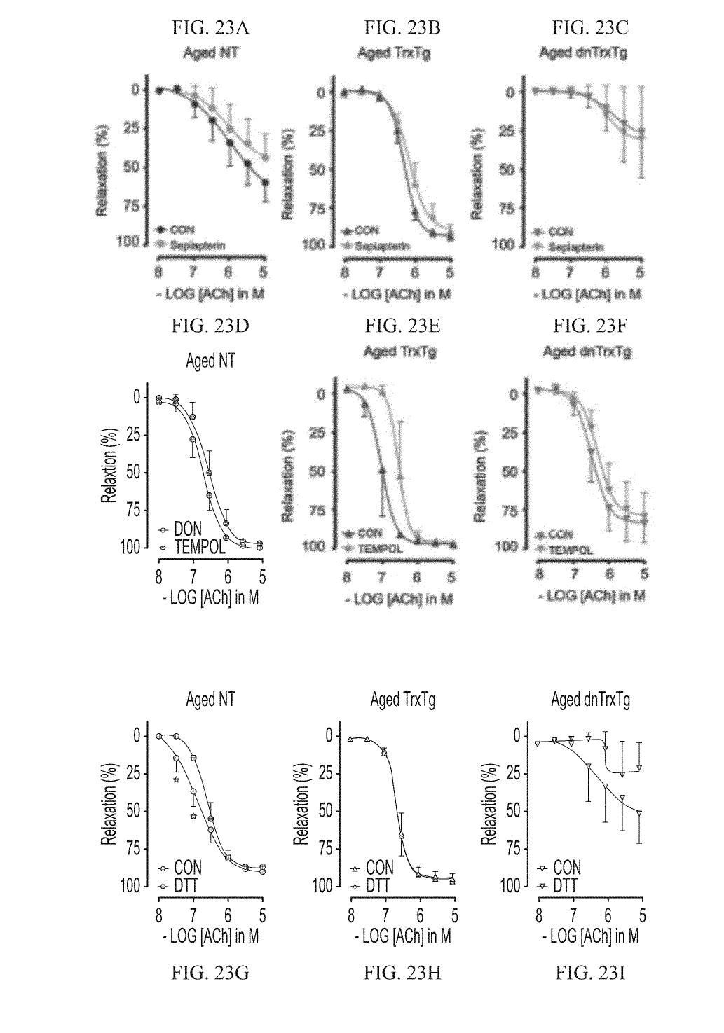

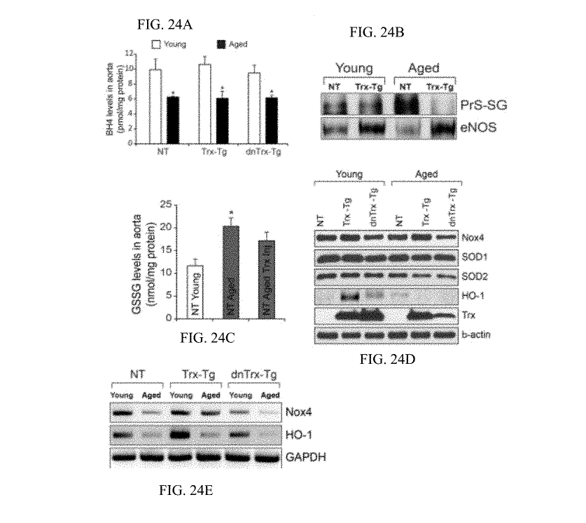

The redox mechanism by which Trx impacts eNOS activation were further studied. Tetrahydrobiopterin (BH₄) is a cofactor of eNOS and decreased BH₄ has been shown in aging mice (Yang et al., supra). The level of BH₄ was decreased in aged mice in all strains compared to young mice (FIG. 24A). However, the levels of BH₄ in young and aged mice remain unchanged, demonstrating no effect of Trx abundance on BH₄ levels. Since BH₄ supplementation has been shown to restore eNOS function in aged mice (Yang et al., supra), we treated aged SMA with sepiapterin, a BH₄ analogue and studied the vascular response to ACh. As shown in FIG. 23A-C, sepiapterin did not restore aging-induced endothelial dysfunction. These data show that although BH₄ levels are decreased in aged SMA, supplementation of only sepiapterin does not provide any protection to restore endothelium-dependent relaxation.

Since TAS of aged NT and dnTrx-Tg mice generates higher levels of O₂.⁻ (FIG. 21C) and decreased levels of NO (FIG. 22A-C) it was determined whether removal of O₂.⁻ by TEMPOL would restore vascular relaxation in aged NT mice. As shown in FIG. 23D-F. TEMPOL did not improve vascular relaxation. These data suggest that in aged vessels, lack of NO due to a dysfunctional eNOS is a major reason of impaired relaxation, but removal of O₂.⁻ produced by eNOS does not improve relaxation. Given the fact that rhTrx improves NO release and increases vascular relaxation in aged mice, we determined the effect of chemical reductant DTT on NO-dependent vascular relaxation in ex vivo myography. As shown in FIG. 23G-I), DTT improved vascular relaxation in aged NT mice, but not in aged dnTrx-Tg mice, demonstrating that reduction of other components of eNOS is critical for restoration of eNOS function, which is sensitive either to Trx or DTT.

The present inventor and colleagues recently showed that eNOS is glutathionylated in response to ischemia-reperfusion injury in the mouse coronary artery (Subramani et al., 2016)). Therefore, we evaluated whether eNOS is S-glutathionylated in aged NT mice. Interestingly, the eNOS S-glutathionylation was increased in mesenteric arteries from aged NT mice, but not in aged Trx-Tg mice. This data suggest that glutathionylation of eNOS occurs in aged mice and high levels of Trx deglutathionylate eNOS and could thus restore eNOS function, because eNOS glutathionylation has been shown to impair its activity irrespective of the amount of BH₄ (CA Chen, Nature 468, 1115-18 (2010)). Consistent with this finding we have recently shown that high levels of Trx is able to deglutathionylate eNOS in vascular endothelial cells in ischemia-reperfusion injury of the coronary artery (Subramani et al., supra). Further, the level of oxidized glutathione (GSSG) was increased in aged NT mice by about 40%, but injection of Trx reduced this increase to 20% (FIG. 24A). Interestingly, we found that the level of Nox4 was decreased in dnTrx-Tg mice, but not in Trx-Tg mice (FIG. 24D). However, the level of SOD1 or SOD2 did not change in young or aged mice vessels (24D). The expression of hemeoxygenase (HO-1) was increased in young Trx-Tg mice, but was almost absent in aged mice of all strains (FIG. 24D).

### Discussion and Conclusions:˜Examples 4-12

In the above examples, the present inventor has demonstrated novel anti-hypertensive efficacy of Trx in age-related hypertension using the newly created Trx-Tg and dnTrx-Tg mice. Further, treatment of aged hypertensive WT mice with rhTrx significantly decreased blood pressure to the level observed in young WT mice, demonstrating therapeutic efficacy of rhTrx. Evidence has been provided that aged mice with Trx overexpression are normotensive due to preservation of in vivo vascular redox state in its reduced form similar to young WT or Trx-Tg mice. In contrast, age-related shift of vessel redox to the oxidized state resulted in a hypertensive phenotype in NT or dnTrx-Tg mice. Further, it was shown that preserved vascular redox state during aging decreases age-related hypertension by the following mechanism(s): 1) maintenance of NO-mediated relaxing responses, 2) preservation of functional NO release, 3) activated Akt-dependent eNOS^{Ser1177} phosphorylation, 4) decreased eNOS-dependent generation of O₂.⁻ in arteries of Trx-Tg mice due to preservation of eNOS function during aging, 5) abrogation of eNOS glutathionylation in the SMA of Trx-Tg mice, and 6) decreased arterial stiffness with improved vascular flow.

There are two major determinants of age-related hypertension, increased arterial stiffness and the loss of arterial relaxing factors. Aging adversely impacts both of these factors resulting in increase in SBP in the elderly. Although large elastic artery stiffening is critical to the development of age-related hypertension, the stiffness of resistance arteries change little with age. Resistance arteries play a crucial role in regulating peripheral vascular resistance, but not so much in elasticity. Hence, we focused on larger elastic arteries, such as the superior mesenteric artery, carotid artery, and thoracic aorta. Consistent with this notion, the present data show that increased SMA remodeling is correlated with high blood pressure in aged mice. In contrast, although pulse wave velocity (PWV) of large elastic arteries in aged mice are greater, it does not correlate with high blood pressure. (The present data demonstrate that high level of Trx in Trx-Tg mice reverses both of these adverse determinants during aging. The endothelium is crucial in sensing the hemodynamic stresses that act on the luminal surface of the arterial wall in the direction of the blood flow. Changes in hemodynamic stresses, such as shear stress (blood flow) and circumferential wall stress (pressure), have been shown to result in structural adaptive arterial responses (P F Davies, Nat Clin Pract Cardiovasc Med 6, 16-26 (2009). NO has been shown to be an important mediator in blood flow-induced arterial remodeling in that it can stimulate structural outward remodeling of the arterial wall (R J et al., Surgery 122, 273-79; discussion 279-280 (1997); F. Tronc et al., Arterioscler Thromb Vasc Biol 16:1256-62 (1996); R D Rudic et al., J Clin Invest 101 731-36 (1998)). In addition, NO has anti-proliferative actions on vascular cells, which may have contributed to the absence of wall thickness increase observed in SMA from aged Trx-Tg mice compared to NT and dnTrx-Tg mice (L Y Deng et al., Clin Exp Hypertens 15, 527-537 (1993); H. Kato et al., Hypertension 28:153-158 (1996)). The present inventor and colleagues recently published that high levels of Trx induces MAP Kinase Kinase 4 (MKK4) activation in endothelial cells (Kundumani-Sridharan, et al., 2015, supra)). Additionally, MKK4 has been shown to be a negative regulator of TGFβ signaling in endothelial cells (L. Davies et al., J Amer Heart Assoc3, e000340 (2014)). Since TGFβ signaling is involved in arterial aging due to MMP-2-TβRII signaling that is implicated in age-associated arterial stiffness, activation of MKK4 would decrease TGFβ-MMP2-TβRII pathway leading to decreased extracellular matrix protein breakdown and decreased arterial stiffness and improved relaxation. Therefore, at the molecular levels Trx-mediated MKK4 signaling may contribute to decreased arterial stiffness in aging.

Endothelial dysfunction plays a major role in age-related hypertension (G. Favero et al., BioMed research international 2014, 801896 (2014)). In large arteries, where NO is the main vasorelaxing factor, the impairment in endothelium-dependent relaxation is manifested by a decrease in NO bioavailability due to uncoupled eNOS protein that produces deleterious O₂.⁻ instead of vasorelaxing factor NO (M. Barton et al., Hypertension 30:817-824 (1997); M R Tschudi et al., J Clin Investig 98: 899-905 (1996); D. Sun et al., Amer J Physiol. Heart and Circ Physiol 286:H2249-2256 (2004)). Aging is a chronic process of oxidation due to aerobic respiration that produces relatively high levels of mitochondrial ROS. Thus, accumulation of oxidative products over the life span of a metazoan accelerates the aging process toward the later part of the life cycle. The present inventor has shown that by increasing the level of Trx from the beginning of life, we could lower the age-related hypertension. Overexpression of Trx minimizes the oxidative modification of redox active proteins such as eNOS and maintains it in a functional manner as shown here. Since Trx undergoes redox cycling and oxidized Trx is continuously regenerated by TrxR using NADPH, augmenting the level of Trx would out compete the chronic oxidation process. For example, in NT mice we observed eNOS dysfunction and increased production of ROS. This age-related dysfunction of eNOS ensues due to oxidation of co-factor BH₄ or glutathionylation of cysteines or both, which are age-dependent in the absence of any other oxidative stress conditions. Although we noted a decrease in BH₄ levels in the SMA of all strains of aged mice, there was no effect of high levels of Trx on the BH₄ levels among NT, Trx-Tg or dnTrx-Tg mice. This is not surprising, in fact Trx is a protein disulfide reductase, so the BH₄ is not a substrate for Trx. Besides, it is now well established that eNOS can be uncoupled irrespective of presence or absence of BH₄ due to glutathionylation of Cys 689 and Cys 908 (Chen et al., supra).

The present results data demonstrate that indeed eNOS is glutathionylated during the aging process, and eNOS remains deglutathionylated due to high levels of Trx in Trx-Tg mice. Thus, high levels of Trx not only prevents eNOS glutathionylation, but could also continually degutathionylate eNOS during the aging process resulting in proper function of eNOS to produce NO, but not O₂.⁻. The present inventor and colleagues recently showed that Trx is an efficient deglutathionylating agent even in the presence of high levels of GSSG in a myocardial ischemia-reperfusion injury model (Subramani et al., supr). In addition, it was observed a consistent decrease in the level of Nox4 in dnTrx-Tg mice, but not in Trx-Tg mice. Further, VAS 2870 was able to decrease ROS only in dnTrx-Tg mice vessels, but not in Trx-Tg, demonstrating that decreased levels of Nox4 may contribute to increased production of O₂.⁻ in aged dnTrx-Tg mice. Although paradoxical, the present data show a protective role of Nox4 in age-related ROS generation consistent with vasoprotective role of Nox4 in NO production, HO-1 generation and H₂O₂ generation as reported in a recent study (K. Schroder et al., Circ Res 110, 1217-1225 (2012)). Therefore, loss of Nox4 expression in aged dnTrx-Tg mice may constitute an underlying mechanism for development of hypertensive phenotype in aged dnTrx-Tg mice.

Trx is not a O₂.⁻ scavenger although it scavenges hydroxyl radicals or singlet oxygen in a redox independent manner ((Das et al., BBRC, 2000)). Further, Trx induces the expression and activity of Sod2 in cells of human or primate origin, but not rodents (Das et al., 1997, supra). However, Trx does restore disulfides or disulfide-containing active sites of enzymes to native thiol state due to direct transfer of electrons. It can also convert mixed disulfides to thiol and the other respective thiolate protein. This property of Trx is critically important in age-related protein oxidation, as Trx can restore proteins to their native state. Sod2 is the major O₂.⁻ detoxifying enzyme in the mitochondrion, and aging has been shown to increase the production of enhanced levels of O₂.⁻. Further, mitochondrial O₂.⁻ has been implicated in hypertension (RR. Nazarewicz et al., Am J Physiol. Heart Circ Physiol 305:H1131-1140 (2013)). However, conventional antioxidants such as Sod1, Sod2 or even Sod1 mimetics such as TEMPOL can only remove the O₂.⁻ that is produced by proteins such as dysfunctional eNOS or NADPH oxidases, but do not possess disulfide reductase activity. Based on this reasoning, Sod or its mimetic would be unable to restore age-related eNOS dysfunction, but can only remove the O₂.⁻ produced by uncoupled eNOS that would prevent further oxidative damage to vascular tissue. Our study showed that high levels of Trx do not allow eNOS to be uncoupled when expressed from the beginning of life. Collectively, the present studies demonstrate that high levels of Trx restore eNOS function to generate NO, but not O₂.⁻.

The present inventor has demonstrated that treatment of aged mice with rhTrx reversed a hypertensive phenotype to normotensive phenotype by restoring eNOS function that lasted for a minimum of 15-20 days. Consistent with this dysfunctional eNOS mechanism, eNOS knockout mice have been reported to show high blood pressure (BN Van Vliet et al., J Physiol 549, 313-25 (2003)). The arteries of rhTrx-treated mice also reversed stiffness and improved endothelial function similar to Trx-Tg mice. According to the present invention, Trx is more potent in blood pressure control in humans compared to rodents due to its induction of Sod2 (Das et al., 1997, supra)). Since rhTrx induces high levels of Sod2 in only cells of primate or human origin, the efficacy of rhTrx in lowering blood pressure and improving endothelial function in aged humans will be much more effective compared to mice models.

In summary, the present invention provides a conceptual advance in exploring intervention strategies that are geared towards reversal of hypertension by pharmacological approaches in the elderly and in other individuals, which will open up new treatment regimens using rhTrx to decrease or eliminate age-related hypertension.

It is believed that any embodiment discussed in this specification can be implemented with respect to any method, kit, reagent, or composition of the invention, and vice versa. Furthermore, the compositions of the invention can be used to achieve methods of the invention. It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context. As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted while one of ordinary skilled in the art will recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

This application is based in part on the contents of the following publication by the present inventor and colleagues (incorporated by reference in its entirety), which is not prior art with respect to the priority dates claimed by this application.
R. H. P. Hilgers, V. Kundumani-Sridharan, J. Subramani, L C Chen, L G Cuello, N J. Rusch, K C Das, Thioredoxin reverses age-related hypertension by chronically improving vascular redox and restoring eNOS function, Sci Transl Med. 9(376) 2017 Feb. 8
1. Y. Higashi, Y. Kihara, K. Noma, Endothelial dysfunction and hypertension in aging. Hypertens. Res. 35, 1039-1047 (2012)
2. D. Susic, E. D. Frohlich, The aging hypertensive heart: A brief update. Nat. Clin. Pract. Cardiovasc. Med. 5, 104-110 (2008)
3. S. Oparil, C. E. Lewis, Should patients with cardiovascular risk factors receive intensive treatment of hypertension to <120/80 mm Hg target? A protagonist view from the SPRINT trial (Systolic Blood Pressure Intervention Trial). Circulation 134, 1308-1310 (2016)
4. E. Pimenta, S. Oparil, Management of hypertension in the elderly. Nat. Rev. Cardiol. 9, 286-296 (2012)
5. E. L. Schiffrin, Oxidative stress, nitric oxide synthase, and superoxide dismutase: A matter of imbalance underlies endothelial dysfunction in the human coronary circulation. Hypertension 51, 31-32 (2008)
6. P. M. Vanhoutte, H. Shimokawa, E. H. C. Tang, M. Feletou, Endothelial dysfunction and vascular disease. Acta Physiol. 196, 193-222 (2009)
7. J. D. Widder, D. Fraccarollo, P. Galuppo, J. M. Hansen, D. P. Jones, G. Ertl, J. Bauersachs, Attenuation of angiotensin II-induced vascular dysfunction and hypertension by overexpression of thioredoxin 2. Hypertension 54, 338-344 (2009)
8. E. G. Lakatta, D. Levy, Arterial and cardiac aging: Major shareholders in cardiovascular disease enterprises: Part I: Aging arteries: A “set up” for vascular disease. Circulation 107, 139-146 (2003)
9. C. Rammos, U. B. Hendgen-Cotta, R. Deenen, J. Pohl, P. Stock, C. Hinzmann, M. Kelm, T. Rassaf, Age-related vascular gene expression profiling in mice. Mech. Ageing Dev. 135, 15-23 (2014)
10. B. M. Kaess, J. Rong, M. G. Larson, N. M. Hamburg, J. A. Vita, D. Levy, E. J. Benjamin, R. S. Vasan, G. F. Mitchell, Aortic stiffness, blood pressure progression, and incident hypertension. JAMA 308, 875-881 (2012)
11. D. Liao, D. K. Arnett, H. A. Tyroler, W. A. Riley, L. E. Chambless, M. Szklo, G. Heiss, Arterial stiffness and the development of hypertension. The ARIC study. Hypertension 34, 201-206 (1999)
12. Y.-M. Yang, A. Huang, G. Kaley, D. Sun, eNOS uncoupling and endothelial dysfunction in aged vessels. Am. J. Physiol. Heart Circ. Physiol. 297, H1829-H1836 (2009)
13. G. P. Dupont, T. P. Huecksteadt, B. C. Marshall, U. S. Ryan, J. R. Michael, J. R. Hoidal, Regulation of xanthine dehydrogenase and xanthine oxidase activity and gene expression in cultured rat pulmonary endothelial cells. J. Clin. Invest. 89, 197-202 (1992)
14. K. Nakazono, N. Watanabe, K. Matsuno, J. Sasaki, T. Sato, M. Inoue, Does superoxide underlie the pathogenesis of hypertension? Proc. Natl. Acad. Sci. U.S.A. 88, 10045-10048 (1991)
15. B. Lassegue, K. K. Griendling, NADPH oxidases: Functions and pathologies in the vasculature. Arterioscler. Thromb. Vasc. Biol. 30, 653-661 (2010)
16. B. Lassegue, A. San Martin, K. K. Griendling, Biochemistry, physiology, and pathophysiology of NADPH oxidases in the cardiovascular system. Circ. Res. 110, 1364-1390 (2012)
17. C. A. Hamilton, M. J. Brosnan, M. McIntyre, D. Graham, A. F. Dominiczak, Superoxide excess in hypertension and aging: A common cause of endothelial dysfunction. Hypertension 37, 529-534 (2001)
18. S. Taddei, A. Virdis, L. Ghiadoni, G. Salvetti, G. Bernini, A. Magagna, A. Salvetti, Age-related reduction of NO availability and oxidative stress in humans. Hypertension 38, 274-279 (2001)
19. B. van der Loo, R. Labugger, J. N. Skepper, M. Bachschmid, J. Kilo, J. M. Powell, M. Palacios-Callender, J. D. Erusalimsky, T. Quaschning, T. Malinski, D. Gygi, V. Ullrich, T. F. Liischer, Enhanced peroxynitrite formation is associated with vascular aging. J. Exp. Med. 192, 1731-1744 (2000)
20. N. Andrawis, D. S. Jones, D. R. Abernethy, Aging is associated with endothelial dysfunction in the human forearm vasculature. J. Am. Geriatr. Soc. 48, 193-198 (2000)
21. D. R. Seals, K. L. Jablonski, A. J. Donato, Aging and vascular endothelial function in humans. Clin. Sci. 120, 357-375 (2011)
22. P. E. Gates, W. D. Strain, A. C. Shore, Human endothelial function and microvascular ageing. Exp. Physiol. 94, 311-316 (2009)
23. M. J. Crabtree, R. Brixey, H. Batchelor, A. B. Hale, K. M. Channon, Integrated redox sensor and effector functions for tetrahydrobiopterin- and glutathionylation-dependent endothelial nitric-oxide synthase uncoupling. J. Biol. Chem. 288, 561-569 (2013)
24. H. Cai, D. G. Harrison, Endothelial dysfunction in cardiovascular diseases: The role of oxidant stress. Circ. Res. 87, 840-844 (2000). 25. S. P. Didion, D. A. Kinzenbaw, L. I. Schrader, F. M. Faraci, Heterozygous CuZn superoxide dismutase deficiency produces a vascular phenotype with aging. Hypertension 48, 1072-1079 (2006)
26. B. S. Fleenor, D. R. Seals, M. L. Zigler, A. L. Sindler, Superoxide-lowering therapy with TEMPOL reverses arterial dysfunction with aging in mice. Aging Cell 11, 269-276 (2012)
27. H. Muniyappa, S. Song, C. K. Mathews, K. C. Das, Reactive oxygen species-independent oxidation of thioredoxin in hypoxia: Inactivation of ribonucleotide reductase and redox-mediated checkpoint control. J. Biol. Chem. 284, 17069-17081 (2009)
28. K. C. Das, P. M. B. Pahl, X.-L. Guo, C. W. White, Induction of peroxiredoxin gene expression by oxygen in lungs of newborn primates. Am. J. Respir. Cell Mol. Biol. 25, 226-232 (2001)
29. A. Holmgren, M. Bjornstedt, Thioredoxin and thioredoxin reductase. Methods Enzymol. 252 199-208 (1995)
30. A. Holmgren, C. Johansson, C. Berndt, M. E. Lorin, C. Hudemann, C. H. Lillig, Thiol redox control via thioredoxin and glutaredoxin systems. Biochem. Soc. Trans 33, 1375-1377 (2005)
31. K. C. Das, C. K. Das, Thioredoxin, a singlet oxygen quencher and hydroxyl radical scavenger: Redox independent functions. Biochem. Biophys. Res. Commun. 277, 443-447 (2000)
32. K. C. Das, Y. Lewis-Molock, C. W. White, Elevation of manganese superoxide dismutase gene expression by thioredoxin. Am. J. Respir. Cell Mol. Biol. 17, 713-726 (1997)
33. A. Holmgren, Thioredoxin. Annu. Rev. Biochem. 54, 237-271 (1985)
34. M. Tanito, H. Nakamura, Y.-W. Kwon, A. Teratani, H. Masutani, K. Shioji, C. Kishimoto, A. Ohira, R. Horie, J. Yodoi, Enhanced oxidative stress and impaired thioredoxin expression in spontaneously hypertensive rats. Antioxid. Redox Signal. 6, 89-97 (2004)
35. M. Matsui, M. Oshima, H. Oshima, K. Takaku, T. Maruyama, J. Yodoi, M. M. Taketo, Early embryonic lethality caused by targeted disruption of the mouse thioredoxin gene. Dev. Biol. 178, 179-185 (1996)
36. K. C. Das, Thioredoxin-deficient mice, a novel phenotype sensitive to ambient air and hypersensitive to hyperoxia-induced lung injury. Am. J. Physiol. Lung Cell. Mol. Physiol. 308, L429-L442 (2015)
37. R. H. P. Hilgers, K. C. Das, Role of in vivo vascular redox in resistance arteries. Hypertension 65, 130-139 (2015)
38. J. E. Oblong, M. Berggren, P. Y. Gasdaska, G. Powis, Site-directed mutagenesis of active site cysteines in human thioredoxin produces competitive inhibitors of human thioredoxin reductase and elimination of mitogenic properties of thioredoxin. J. Biol. Chem. 269, 11714-11720 (1994)
39. H. Smulyan, M. E. Safar, Systolic blood pressure revisited. J. Am. Coll. Cardiol. 29, 1407-1413 (1997)
40. R. M. K. W. Lee, C. R. Triggle, Morphometric study of mesenteric arteries from genetically hypertensive Dahl strain rats. Blood Vessels 23, 199-224 (1986)
41. V. Kundumani-Sridharan, J. Subramani, K. C. Das, Thioredoxin activates MKK4-NFkB pathway in a redox-dependent manner to control manganese superoxide dismutase gene expression in endothelial cells. J. Biol. Chem. 290, 17505-17519 (2015)
42. Q.-A. Sun, D. T. Hess, B. Wang, M. Miyagi, J. S. Stamler, Off-target thiol alkylation by the NADPH oxidase inhibitor 3-benzyl-7-(2-benzoxazolyl)thio-1,2,3-triazolo[4,5-d]pyrimidine (VAS2870). Free Radic. Biol. Med. 52, 1897-1902 (2012)
43. J. Subramani, V. Kundumani-Sridharan, R. H. P. Hilgers, C. Owens, K. C. Das, Thioredoxin uses a GSH-independent route to deglutathionylate endothelial nitric-oxide synthase and protect against myocardial infarction. J. Biol. Chem. 291, 23374-23389 (2016)
44. C.-A. Chen, T.-Y. Wang, S. Varadharaj, L. A. Reyes, C. Hemann, M. A. H. Talukder, Y.-R. Chen, L. J. Druhan, J. L. Zweier, 5-glutathionylation uncouples eNOS and regulates its cellular and vascular function. Nature 468, 1115-1118 (2010)
45. P. F. Davies, Hemodynamic shear stress and the endothelium in cardiovascular pathophysiology. Nat. Clin. Pract. Cardiovasc. Med. 6, 16-26 (2009)
46. R. J. Guzman, K. Abe, C. K. Zarins, Flow-induced arterial enlargement is inhibited by suppression of nitric oxide synthase activity in vivo. Surgery 122, 273-280 (1997)
47. F. Tronc, M. Wassef, B. Esposito, D. Henrion, S. Glagov, A. Tedgui, Role of NO in flow-induced remodeling of the rabbit common carotid artery. Arterioscler. Thromb. Vasc. Biol. 16, 1256-1262 (1996)
48. R. D. Rudic, E. G. Shesely, N. Maeda, O. Smithies, S. S. Segal, W. C. Sessa, Direct evidence for the importance of endothelium-derived nitric oxide in vascular remodeling. J. Clin. Invest. 101, 731-736 (1998).
49. L. Y. Deng, G. Thibault, E. L. Schiffrin, Effect of hypertension induced by nitric oxide synthase inhibition on structure and function of resistance arteries in the rat. Clin. Exp. Hypertens. 15, 527-537 (1993)
50. H. Kato, J. Hou, A. V. Chobanian, P. Brecher, Effects of angiotensin II infusion and inhibition of nitric oxide synthase on the rat aorta. Hypertension 28, 153-158 (1996)
51. L. Davies, J. Jin, W. Shen, H. Tsui, Y. Shi, Y. Wang, Y. Zhang, G. Hao, J. Wu, S. Chen, J. A. Fraser, N. Dong, V. Christoffels, U. Ravens, C. L.-H. Huang, H. Zhang, E. J. Cartwright, X. Wang, M. Lei, Mkk4 is a negative regulator of the transforming growth factor beta 1 signaling associated with atrial remodeling and arrhythmogenesis with age. J. Am. Heart Assoc. 3, e000340 (2014)
52. G. Favero, C. Paganelli, B. Buffoli, L. F. Rodella, R. Rezzani, Endothelium and its alterations in cardiovascular diseases: Life style intervention. Biomed. Res. Int. 2014, 801896 (2014)
53. M. Barton, F. Cosentino, R. P. Brandes, P. Moreau, S. Shaw, T. F. Liischer, Anatomic heterogeneity of vascular aging: Role of nitric oxide and endothelin. Hypertension 30, 817-824 (1997)
54. M. R. Tschudi, M. Barton, N. A. Bersinger, P. Moreau, F. Cosentino, G. Noll, T. Malinski, T. F. Liischer, Effect of age on kinetics of nitric oxide release in rat aorta and pulmonary artery. J. Clin. Invest. 98, 899-905 (1996)
55. D. Sun, A. Huang, E. H. Yan, Z. Wu, C. Yan, P. M. Kaminski, T. D. Oury, M. S. Wolin, G. Kaley, Reduced release of nitric oxide to shear stress in mesenteric arteries of aged rats. Am. J. Physiol. Heart Circ. Physiol. 286, H2249-H2256 (2004)
56. K. Schroder, M. Zhang, S. Benkhoff, A. Mieth, R. Pliquett, J. Kosowski, C. Kruse, P. Luedike, U. R. Michaelis, N. Weissmann, S. Dimmeler, A. M. Shah, R. P. Brandes, Nox4 is a protective reactive oxygen species generating vascular NADPH oxidase. Circ. Res. 110, 1217-1225 (2012)
57. R. R. Nazarewicz, A. E. Dikalova, A. Bikineyeva, S. I. Dikalov, Nox2 as a potential target of mitochondrial superoxide and its role in endothelial oxidative stress. Am. J. Physiol. Heart Circ. Physiol. 305, H1131-H1140 (2013)
58. B. N. Van Vliet, L. L. Chafe, J.-P. Montani, Characteristics of 24 h telemetered blood pressure in eNOS-knockout and C57Bl/6J control mice. J. Physiol. 549, 313-325 (2003).

All publications and patent applications mentioned in the specification are indicative of the level of skill of those skilled in the art to which this invention pertains. The references cited above are all incorporated by reference herein in their entirety, whether specifically incorporated or not. Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A pharmaceutical composition for treating a cardiovascular disorder comprising:
(a) thioredoxin-1 (Trx1) protein (SEQ ID NO:1);
(b) a therapeutically active peptide of (a);
(c) a therapeutically active conservative amino acid sequence variant of (a) or of (b)
(d) a therapeutically active functional derivative of (a) or (b); or
(e) a combination of any one or more of (a)-(d),
in a pharmaceutically acceptable carrier or excipient

2. The pharmaceutical composition of claim 1 wherein the peptide of (b) is a peptide of the sequence SEQ ID NO:5 to SEQ ID NO:38).

3. The pharmaceutical composition of claim 1, wherein the pharmaceutical carrier is one that is adapted for intramuscular, intraperitoneal, intravenous, or subcutaneous administration.

4. The pharmaceutical composition of claim 1, wherein the cardiovascular disorder is a hypertensive disorder.

5. The pharmaceutical composition of claim 1 wherein the Trx1 protein is recombinant human Trx1 (rhTrx1).

6. A pharmaceutical composition for (i) inhibiting aging-induced loss of endothelial cell function, (ii) preserving endothelial nitric oxide synthase (eNOS) protein function or (iii) activating eNOS protein function in a subject, comprising
(a) Trx-1 protein (SEQ ID NO:1)
(b) a pharmaceutically active peptide of (a)
(c) a pharmaceutically active conservative amino acid conservative amino acid sequence variant of (a) or (b)
(d) a functional derivative of (a or (b)
(e) a Trx1 system upregulator, or
(f) a Trx1 system activator,
in a pharmaceutical carrier.

7. A method for ameliorating one or more symptom of a cardiovascular disorder in a subject in need thereof comprising administering to the subject a therapeutically effective amount of a pharmaceutical composition according to claim 1, thereby ameliorating one or more symptom of said cardiovascular disorder.

8. The method of claim 7 wherein the cardiovascular disorder is a hypertensive disorder.

9. The method of claim 7, wherein the pharmaceutical carrier is one that is adapted for intramuscular, intraperitoneal, intravenous, or subcutaneous administration.

10. The method of claim 9 wherein the pharmaceutical carrier is adapted for intravenous administration.

11. The method of claim 6, wherein the pharmaceutical composition is administered every 0.5, 1, 2, 3, 4, 5, 6, or more months.

12. A method for treating a subject having a cardiovascular disorder comprising administering to the subject a therapeutically effective amount of (a) a Trx1 system upregulator or activator, and/or (b) an agent that causes depletion of nitric oxide in the subject's body.

13. The method of claim 12 wherein the cardiovascular disorder is a hypertensive disorder.

14. The method of claim 12, wherein the pharmaceutical carrier is one that is adapted for intramuscular, intraperitoneal, intravenous, or subcutaneous administration.

15. A method for (i) inhibiting aging-induced loss of endothelial cell function, (ii) preserving endothelial nitric oxide synthase (eNOS) protein function or (iii) activating eNOS protein function in a subject, comprising administering to the subject an effective amount of a pharmaceutical composition according to claim 6, thereby inhibiting the aging-induced loss of endothelial cell function or preserving or activating eNOS protein function.

16. The method according to claim 15 for preserving eNOS protein function in said subject.

17. The method according to claim 15 for activating eNOS protein function in said subject.

18. The method of claim 15, wherein the pharmaceutical carrier is one that is adapted for intramuscular, intraperitoneal, intravenous, or subcutaneous administration.

19. The method of claim 15 wherein the subject suffers from a cardiovascular disorder.

20. The method of claim 19 wherein the cardiovascular disorder is a hypertensive disorder.
